SEQUENCE LISTING (44 sequences)

SEQ ID NO: 1 - DNA, 19 bp, synthetic construct
  source 1..19: mol_type = other DNA
agaaaagtgccgtgggaac

SEQ ID NO: 2 - DNA, 19 bp, synthetic construct
  source 1..19: mol_type = other DNA
tgttgaggtttccccgaat

SEQ ID NO: 3 - DNA, 20 bp, synthetic construct
  source 1..20: mol_type = other DNA
ctcggatgaggcaggctgtg

SEQ ID NO: 4 - DNA, 20 bp, synthetic construct
  source 1..20: mol_type = other DNA
tacagtccgggtgcccatca

SEQ ID NO: 5 - DNA, 20 bp, synthetic construct
  source 1..20: mol_type = other DNA
gctccatccttctccacggc

SEQ ID NO: 6 - DNA, 20 bp, synthetic construct
  source 1..20: mol_type = other DNA
gcatttgcccacagcatcgg

SEQ ID NO: 7 - DNA, 20 bp, synthetic construct
  source 1..20: mol_type = other DNA
catccagtacgccgtcaccc

SEQ ID NO: 8 - DNA, 20 bp, synthetic construct
  source 1..20: mol_type = other DNA
gtggctatcgtgggccactc

SEQ ID NO: 9 - DNA, 20 bp, synthetic construct
  source 1..20: mol_type = other DNA
gtccctctatgcctctggac

SEQ ID NO: 10 - DNA, 20 bp, synthetic construct
  source 1..20: mol_type = other DNA
aaggaatagccacgctcagt

SEQ ID NO: 11 - DNA, 20 bp, synthetic construct
  source 1..20: mol_type = other DNA
tggaaacccgaggtatgctt

SEQ ID NO: 12 - DNA, 20 bp, synthetic construct
  source 1..20: mol_type = other DNA
cattgcattgcacgtcatcg

SEQ ID NO: 13 - DNA, 20 bp, synthetic construct
  source 1..20: mol_type = other DNA
ccctcattcccttgttgcag

SEQ ID NO: 14 - DNA, 20 bp, synthetic construct
  source 1..20: mol_type = other DNA
taggaggacgaggacacaga

SEQ ID NO: 15 - DNA, 20 bp, synthetic construct
  source 1..20: mol_type = other DNA
tcagtggccaatggaagact

SEQ ID NO: 16 - DNA, 20 bp, synthetic construct
  source 1..20: mol_type = other DNA
aacgtcatactcgagcccat

SEQ ID NO: 17 - DNA, 20 bp, synthetic construct
  source 1..20: mol_type = other DNA
aagggcaagaaatgcagcaa

SEQ ID NO: 18 - DNA, 20 bp, synthetic construct
  source 1..20: mol_type = other DNA
ccgcatcttcacagttctgg

SEQ ID NO: 19 - DNA, 20 bp, synthetic construct
  source 1..20: mol_type = other DNA
agtgcatccggacacctaaa

SEQ ID NO: 20 - DNA, 20 bp, synthetic construct
  source 1..20: mol_type = other DNA
gcatttgaactccactggca

SEQ ID NO: 21 - DNA, 20 bp, synthetic construct
  source 1..20: mol_type = other DNA
tcctcccgttcacctacaac

SEQ ID NO: 22 - DNA, 20 bp, synthetic construct
  source 1..20: mol_type = other DNA
acaccacagatgtccgtctt

SEQ ID NO: 23 - DNA, 20 bp, synthetic construct
  source 1..20: mol_type = other DNA
gtggagaacctggaccaaga

SEQ ID NO: 24 - DNA, 20 bp, synthetic construct
  source 1..20: mol_type = other DNA
taccgggctttccaattcct

SEQ ID NO: 25 - DNA, 20 bp, synthetic construct
  source 1..20: mol_type = other DNA
aaacgaggctaggcagaagt

SEQ ID NO: 26 - DNA, 20 bp, synthetic construct
  source 1..20: mol_type = other DNA
tggacaatgtggtggttgtg

SEQ ID NO: 27 - DNA, 20 bp, synthetic construct
  source 1..20: mol_type = other DNA
agtctttgaagggcagcagt

SEQ ID NO: 28 - DNA, 20 bp, synthetic construct
  source 1..20: mol_type = other DNA
agagctccacagacaaggac

SEQ ID NO: 29 - DNA, 20 bp, synthetic construct
  source 1..20: mol_type = other DNA
gagctcttcgaagggaagga

SEQ ID NO: 30 - DNA, 21 bp, synthetic construct
  source 1..21: mol_type = other DNA
catagaggacaactccaaggc

SEQ ID NO: 31 - DNA, 20 bp, synthetic construct
  source 1..20: mol_type = other DNA
ctcttccagccttccttcct

SEQ ID NO: 32 - DNA, 20 bp, synthetic construct
  source 1..20: mol_type = other DNA
agcactgtgttggcgtacag

SEQ ID NO: 33 - DNA, 20 bp, synthetic construct
  source 1..20: mol_type = other DNA
ccgcctcttccacaaatcag

SEQ ID NO: 34 - DNA, 20 bp, synthetic construct
  source 1..20: mol_type = other DNA
gtagggcagttccaggatgt

SEQ ID NO: 35 - DNA, 20 bp, synthetic construct
  source 1..20: mol_type = other DNA
ggtacagggtgacctactcg

SEQ ID NO: 36 - DNA, 20 bp, synthetic construct
  source 1..20: mol_type = other DNA
gggctggctctccatatcat

SEQ ID NO: 37 - DNA, 20 bp, synthetic construct
  source 1..20: mol_type = other DNA
cagcaagtacctcacatgcc

SEQ ID NO: 38 - DNA, 20 bp, synthetic construct
  source 1..20: mol_type = other DNA
tttcttgggtacggcttcct

SEQ ID NO: 39 - DNA, 20 bp, synthetic construct
  source 1..20: mol_type = other DNA
gcgctgcatctcggaagagt

SEQ ID NO: 40 - DNA, 20 bp, synthetic construct
  source 1..20: mol_type = other DNA
tggtggcattgcagtccctg

SEQ ID NO: 41 - DNA, 20 bp, synthetic construct
  source 1..20: mol_type = other DNA
gccctttgtggcctccagtt

SEQ ID NO: 42 - DNA, 20 bp, synthetic construct
  source 1..20: mol_type = other DNA
tgcgatgagcccaaatcccc

SEQ ID NO: 43 - DNA, 20 bp, synthetic construct
  source 1..20: mol_type = other DNA
ctggtggaggggctgtacct

SEQ ID NO: 44 - DNA, 20 bp, synthetic construct
  source 1..20: mol_type = other DNA
ttcccggagctcaagtccca

(19) United States
(12) Patent Application Publication
(10) Pub. No.: US 2026/0306463 A1
(43) Pub. Date: Oct. 8, 2026

(54) SIK INHIBITORS TO TREAT CHRONIC KIDNEY DISEASE

(71) Applicant: The General Hospital Corporation, Boston, MA (US)

(72) Inventors: Marc N. Wein, Boston, MA (US); Sung-Hee Yoon, Boston, MA (US); Michael Mannstadt, Boston, MA (US)

(21) Appl. No.: 19/481,669

(22) PCT Filed: May 10, 2024
(86) PCT No.: PCT/US2024/028951

§ 371 Date: Nov. 5, 2025

Related U.S. Application Data
(60) Provisional application No. 63/501,157, filed on May 10, 2023.

(51) Int. Cl. A61K 31/437; A61K 31/7105; A61K 45/06; A61P 13/12

(52) U.S. Cl. CPC A61K 31/437; A61K 31/7105; A61K 45/06; A61P 13/12

(57) ABSTRACT

Provided herein are methods for treating chronic kidney disease (CKD) in a subject in need thereof by administering to the subject a therapeutically effective amount of an inhibitor of one or more salt inducible kinases (SIKs). Also provided herein are inhibitors of one or more SIKs for use in treating, reducing the risk of occurrence, or delaying the occurrence of CKD in a subject. In some embodiments, the subject does not have acute kidney injury (AKI). In some embodiments, the subject does not have CKD Mineral and Bone Disorder (CKD-MBD). In some embodiments, the subject has early-stage CKD or intermediate-stage CKD.

### CLAIM OF PRIORITY

This application claims the benefit of U.S. Provisional Application Ser. No. 63/501,157, filed on May 10, 2023. The entire contents of the foregoing are incorporated herein by reference.

### FEDERALLY SPONSORED RESEARCH OR DEVELOPMENT

This invention was made with Government support under Grant Nos. DK116716 and DK011794 awarded by the National Institutes of Health. The Government has certain rights in the invention.

### SEQUENCE LISTING

This application contains a Sequence Listing that has been submitted electronically as an XML file named “29539-0754WO1.XML.” The XML file, created on May 9, 2024, is 39,721 bytes in size. The material in the XML file is hereby incorporated by reference in its entirety.

### TECHNICAL FIELD

Described herein are methods for treating chronic kidney disease, using agents that target salt inducible kinases (SIKs).

### BACKGROUND

Chronic kidney disease (CKD) is the 16th leading cause of years of life lost worldwide. Described as a persistent abnormality in kidney structure or function (e.g., glomerular filtration rate (GFR)<60 mL/min/1.73 m2 or albuminuria≥30 mg per 24 hours) for more than 3 months, CKD affects 8% to 16% of the population worldwide (Chen et al. Chronic Kidney Disease Diagnosis and Management: A Review. JAMA. 2019, 322(13):1294-1304).

### SUMMARY

Provided herein are methods for treating chronic kidney disease (CKD) in a subject in need thereof by administering to the subject a therapeutically effective amount of an inhibitor of one or more salt inducible kinases (SIKs). Also provided herein are inhibitors of one or more SIKs for use in treating, reducing the risk of occurrence, or delaying the occurrence of CKD in a subject.

In some embodiments, the subject does not have acute kidney injury (AKI).

In some embodiments, the subject does not have CKD Mineral and Bone Disorder (CKD-MBD). In some embodiments, the subject has early-stage CKD or intermediate-stage CKD.

In some embodiments, the methods reduce one or more symptoms of CKD in the subject, as compared to before the inhibitor is administered to the subject. In some embodiments, the methods result in one or more of reduced expression of one or more markers of renal dysfunction in the subject; reduced expression of one or more fibrosis markers in the subject: reduced expression of one or more renal injury markers in the subject; reduced expression of one or more proximal tubule injury markers in the subject; increased expression of one or more markers of intact proximal epithelial cells in the subject; preservation of epithelial cell integrity in the subject: increased number of healthy proximal tubule cells in the subject; and/or improved renal function in the subject, as compared to before the inhibitor is administered to the subject. In some embodiments, the markers of renal dysfunction comprises one or more of creatinine, BUN, proteinuria, and cystatin C; the fibrosis marker comprises one or more of alpha-smooth muscle actin (αSMA), Collal, and Vimentin; the renal injury marker comprises one or more of Col3al, Vim, Serpinel, Pai-1, Fibronectin-1, and Col8al: the proximal tubule injury marker comprises p21; and/or the marker of intact proximal tubular epithelial cells comprises one or more of Lrp2, Atplal, Slc34al, Hnf4a, and Slc17a2.

In some embodiments, the methods further comprise administering to the subject one or more additional therapeutic agents. In some embodiments, the one or more additional therapeutic agents comprises an ACE inhibitor, an angiotensin II receptor blocker, a lipid control agent, a D-vitamin supplementation, a phosphate control agent, an anemia control agent, an acidosis prevention agent, and/or a uric acid control agent.

Also provided herein are methods for reducing the risk of occurrence or delaying the occurrence of CKD in a subject in need thereof, by administering to the subject a therapeutically effective amount of an inhibitor of one or more SIKs.

In some embodiments, the subject is at risk for developing CKD. In some embodiments, the subject has one or more of diabetes, high blood pressure, heart disease, family history of CKD, and obesity.

In some embodiments, one or more doses of the inhibitor is administered to the subject. In some embodiments, the inhibitor is administered to the subject multiple times at daily, weekly, or monthly intervals.

In some embodiments, the inhibitor is an inhibitor of one or more of SIK2 and SIK3.

In some embodiments, the inhibitor is a compound that is a selective SIK2/SIK3 inhibitor, or a pharmaceutically acceptable salt thereof. In some embodiments, the inhibitor is 2-[2.6-Dimethoxy-4-[7-(1-methylpyrazol-4-yl)imidazo[1,2-a]pyridin-3-yl]phenyl]-5-ethyl-1,3,4-oxadiazole, or a pharmaceutically acceptable salt thereof. In some embodiments, the inhibitor is a compound having the structure:

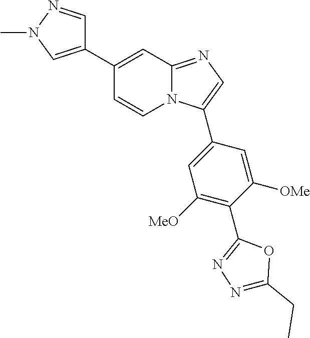 [SMILES: CCc1nnc(-c2c(OC)cc(-c3cnc4cc(-c5cnn(C)c5)ccn34)cc2OC)o1]

or a pharmaceutically acceptable salt thereof.

In some embodiments, the inhibitor is an inhibitory nucleic acid that targets a SIK2/SIK3 transcript. In some embodiments, the inhibitory nucleic acid is selected from the group consisting of antisense oligonucleotides, small interfering RNAs (siRNAs), and small hairpin RNAs (shRNAs). In some embodiments, the inhibitory nucleic acid is modified. In some embodiments, the inhibitory nucleic acid comprises a modified backbone. In some embodiments, the modified backbone comprises an amide or morpholino backbone. In some embodiments, the inhibitory nucleic acid comprises one or more modified nucleosides. In some embodiments, the inhibitory nucleic acid comprises at least one locked nucleoside.

In some embodiments, the subject is a human.

Unless otherwise defined, all technical and scientific terms used herein have the same meaning as commonly understood by one of ordinary skill in the art to which this invention belongs. Methods and materials are described herein for use in the present invention: other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1E. SK-124 (SIK2/SIK3 inhibitor) treatment improves renal function in mice with adenine-induced chronic kidney disease. (A) Study design: Female CD1 mice (n=8/group) were treated with control or 0.2% adenine diet starting at 6 weeks of age along with vehicle or SK-124 (40 mg/kg) treatment by oral gavage once daily. Bottom: body weight of mice in the indicated treatment groups. CKD-induced weight loss is prevented by SK-124 treatment. (B, C) After 6 weeks of treatment, the indicated analytes were measured in fasting serum or urine. All CKD-induced changes were prevented by SK-124 treatment. (D) H+E-stained kidney sections were assessed on a blinded basis by a pathologist for tubulointerstitial damage. (E) Whole kidney RNA from the indicated groups was analyzed by RT-qPCR for the indicated proximal tubule marker gene. For all panels in all figures, 1-way ANOVA was performed with Dunnett's posthoc analysis to analyze differences between treatment groups, p values less than 0.05 are shown in graph.

FIGS. 2A-2C. SIK isoform expression in adenine-induced chronic kidney disease. (A) Kidney protein lysates were isolated and analyzed by imnunoblotting for SIK2 (top) and GAPDH (bottom). SIK2 protein levels are increased in adenine diet. (B) Whole kidney RNA from the indicated groups was analyzed by RT-qPCR for the indicated SIK isoform. (C) Kidney sections from control and CKD (6 weeks adenine diet) were stained using RNAscope (darker shade) for the indicated SIK isoform.

FIGS. 3A-3C. SK-124 treatment reduces fibrosis in adenine-induced chronic kidney disease. (A) Representative trichrome stained sections from the indicated groups. Darker staining indicates fibrosis which is increased in vehicle-treated adenine diet mice and reduced by SK-124 treatment. (B) Kidney protein lysates were isolated and analyzed by immunoblotting for alpha-smooth muscle actin (αSMA) (top) and GAPDH (bottom). (C) Whole kidney RNA from the indicated groups was analyzed by RT-qPCR for the indicated fibrosis marker.

FIGS. 4A-4D. SK-124 treatment, but not once daily parathyroid hormone injections, improves kidney function in mice with adenine diet-induced chronic kidney disease. (A) Study design: Female CD1 mice (n=8/group) were treated with control or 0.2% adenine diet starting at 6 weeks of age along with vehicle, PTH (80 mcg/kg/d, IP injections) or SK-124 (40 mg/kg) treatment by oral gavage once daily. Mice were treated for 8 weeks. (B, C) After 8 weeks of treatment, the indicated serum analyte was measured. (D) Whole kidney RNA from the indicated groups was analyzed by RT-qPCR for the indicated fibrosis marker gene.

FIGS. 5A-5C. SIK2/SIK3 gene deletion improves renal function and reduces fibrosis in mice with adenine diet-induced chronic kidney disease. (A) Study design: control or double knockout mice were treated with tamoxifen at 5 weeks of age followed by adenine or control diet one week later. After 3 weeks on adenine diet, mice were sacrificed. (B) Serum BUN levels were increased in adenine diet-induced CKD, and reduced in double SIK2/SIK3 knockout mice (DKO) compared to control animals. (C) Whole kidney RNA from the indicated groups was analyzed by RT-qPCR for the indicated fibrosis marker gene.

FIGS. 6A-6F. The effects of SK-124 treatment at the transcriptomic level in adenine-induced mouse chronic kidney disease. RNA-seq was performed on whole kidney RNA samples (n=7/group) from FIG. 1. Volcano plot show gene regulated (>2-fold change, p(adj)<0.05) by CKD (versus control diet, A) or in CKD by SK-124 (versus vehicle control, B). In Volcano plot, representative fibrosis markers (Col3a1, Vim, Serpinel) and proximal tubule integrity (Lrp2. Atplai, and Hnf4a) are shown. Scatterplot (C) and heatmap (D) showing integration of individual RNA-seq comparisons. Genes that are regulated in CKD (versus control diet) are conversely regulated by SK-124 (versus vehicle control). (E) Single cell RNA-sequencing was performed on dissociated kidneys from adenine diet fed mice treated with vehicle or SK-124 (n=4/group). UMAP plot shows distinct clusters of cells based on transcriptomic profiles. (F) Relative cellularity of each annotated cluster is shown in pie chart format for adenine+vehicle (CKD-Veh) or adenine+SK-124 (CKD-SK-124) mice. SK-124 treatment reduces fibroblast cluster cellularity and increases numbers of healthy epithelial cells.

FIG. 7. SK-124 preserves human kidney organoid integrity in cisplatin injury model. Human kidney organoids derived from H9 embryonic stem cells were treated with intermittent cisplatin (5 separate treatments) based on published protocols along with the indicated agent. Organoids were cryo-sectioned and immuno-stained with the indicated markers. The top panels show low power images and the bottom panels focus on regions of proximal tubule cells. The graph measures the mean diameter of organoids in the control (no cisplatin), cisplatin+DMSO, and cisplatin+SK-124 treatment groups. Overall, SK-124, but not PTH or calcitriol, preserves organoid cellularity and size in response to cisplatin injury.

FIG. 8. SK-124 treatment restores gene expression changes in cisplatin-injured human kidney organoids. RNA was isolated from organoids treated as in FIG. 7 for RT-qPCR for the indicated genes. Injury markers (Pai-1, Fibronectin-1, and Co18a1) are induced by cisplatin treatment and reduced by SK-124. In contrast, the proximal tubule marker Lrp2 is reduced by cisplatin treatment and rescued by SK-124.

FIG. 9. SK-124 treatment protects from cisplatin-induced P21 induction in human kidney organoids. Organoids were treated with the indicated number of cisplatin cycles in the presence of DMSO vehicle (top) or SK-124 (bottom). Organoids were then processed for P21 immunofluorescent microscopy. Cisplatin exposure induces P21 expression, which is reduced by concurrent SK-124 treatment.

FIGS. 10A-10B. SK-124 treatment restores gene expression changes in cisplatin-injured human kidney organoids. RNA was isolated from organoids treated as in FIG. 9 with the indicated number of cisplatin cycles plus DMSO control (“Cis”) or SK-124 (10 μM; “Cis+SK”) for RT-qPCR. (A) Proximal tubule integrity markers (Lrp2 and Sic]7a2) are progressively reduced by cisplatin but restored by co-treatment with SK-124. (B) Injury/fibrosis markers (FN-1, Co/8a1, and Pai-1) were induced by cisplatin and suppressed by SK-124.

FIG. 11. SK-124 protects human kidney organoids from cisplatin injury in a dose-dependent manner. Organoids were treated with five cisplatin cycles in the presence of the indicated doses of SK-124. Subsequently, organoids were immunostained to reveal cellular composition and injury markers P21 (top panels) and gammaH2AX (bottom panels). These markers are induced by cisplatin injury but reduced by SK-124 co-treatment in a dose-dependent manner. Proximal tubule cells are labeled with LTL staining and are reduced by cisplatin treatment and restored by SK-124 co-treatment.

FIGS. 12A-12B. SK-124 protects human kidney organoids from cisplatin injury in a dose-dependent manner. RNA was isolated from organoids treated as in FIG. 11 for RT-qPCR. (A) Proximal tubule marker genes are reduced by cisplatin and increased by SK-124. (B) Fibrosis/injury marker genes are induced by cisplatin and reduced by SK-124.

FIGS. 13A-13E. Transcriptomic effects of SK-124 in cisplatin-induced human kidney organoid model. RNA-seq was performed on kidney organoid RNA samples (n=4/group) from FIG. 8. Volcano plot show gene regulated (>2-fold change, p(adj)<0.05) by cisplatin (versus vehicle, A) or within cisplatin-treated organoids by SK-124 (versus DMSO control, B). In Volcano plot, representative fibrosis/injury markers (Col8a1, Fni, Serpinel) and proximal tubule integrity (Lrp2 and Hnf4a) are shown. Scatterplot (C) and heatmap (D) showing integration of individual RNA-seq comparisons. Genes that are regulated in cisplatin (versus control) are conversely regulated by SK-124 in the setting of injury (versus DMSO control). (E) Top: Venn overlap analysis of genes that were both induced by injury and suppressed by SK-124 in human kidney organoids reveals core group of 419 conserved injury induced transcripts that are ‘rescued’ by SIK2/SIK3 inhibitor treatment. Bottom: Venn overlap analysis of genes that were both reduced by injury and restored by SK-124 in human kidney organoids reveals core group of 373 conserved transcripts whose defective expression is ‘rescued’ by SIK2/SIK3 inhibitor treatment in both CKD models.

FIGS. 14A-14E. SIK pathway controls YAP/TAZ-dependent myofibroblast activation in kidney Gli1 cells. KGlil cells were treated as indicated for 48 hours followed by (A) immunoblotting and (B) RT-qPCR. (C) KGlil cells were treated±TGFß (5 ng/mL) or verteporfin (1 μM) for 48 hours followed by imnunoblotting. (D) Luciferase assay from cells were transfected with a YAP/TAZ responsive reporter [2]. SIK inhibitor and forskolin both block basal and TGFß-stimulated-YAP/TAZ reporter activity. (E) Top, KGlil cells were treated for 24 hours as indicated followed by immunoblotting. Bottom, kidney lysates from adenine diet CKD mice were analyzed by immunoblotting as indicated.

### DETAILED DESCRIPTION

Novel therapies are needed to prevent and treat the development of chronic kidney disease (1). Salt inducible kinases (SIKs) are a broadly expressed subfamily within the AMP-activated protein kinases (AMPK) family (2, 3). We have reported critical roles for SIK2 and SIK3 downstream of parathyroid hormone signaling in bone (4, 5) and kidney (6), and have developed pharmacologic tools to study their function (7). Based on our findings that inhibiting SIKs stimulates 1.25-(OH)₂-vitamin D synthesis in the proximal tubule (6), we designed experiments to interrogate if SIK inhibitors have any therapeutic benefit in mineral/bone derangements (MBD). Unexpectedly, while exploring the therapeutic effect of SIK inhibitors on MBD, we observed dramatic renal-protective function of SIK2/SIK3 inhibitors in mouse chronic kidney disease-mineral and bone disorder (CKD-MBD) model and human kidney organoid injury model.

Salt inducible kinases (SIKs) are cytoplasmic AMPK family kinases. Although SIK1 is induced by dietary salt intake in the adrenal gland (8), SIK2 and SIK3 are broadly expressed and are not regulated by salt. Rather, SIK2/SIK3 activity is induced by kinase domain phosphorylation by the upstream activating kinase LKB1 and inhibited through an allosteric mechanism by cAMP/protein kinase A (PKA)-dependent phosphorylation (9-11). We previously used pharmacologic and genetic tools to define critical roles for these kinases downstream of parathyroid hormone action in bone and kidney (4-6, 12). Canonical SIK substrates include class IIa histone deacetylases (HDACs) and cAMP-regulated transcriptional coactivator (CRTC) proteins. When cellular cAMP levels are low, these substrates are phosphorylated are retained in the cytoplasm. Upon cAMP signaling and PKA-mediated SIK inhibition, SIK substrate phosphorylation is reduced, and these proteins translocate into the nucleus to regulate cell type-specific target gene expression.

Notably, this ‘canonical’ SIK mechanism operates downstream of several GPCR systems to control gene expression in response to cAMP signaling (3, 13, 14).

Identifying specific pharmacologic SIK inhibitors has been challenging because these kinases, like many unrelated tyrosine kinases, bear small threonine residues at the gatekeeper site (11). Recently, structure-based drug design has identified ‘next generation’ compounds with improved specificity and drug-like features (15-18). We recently reported the synthesis and characterization of SK-124 and related analogs, a series of SIK2/SIK3-selective inhibitor with drug-like features and PTH-like effects in vivo in bone and kidney (7). While SIK2 inhibitors have progressed into clinical trials for patients with SIK2-overexpressing ovarian cancer (15), the therapeutic potential of SIK inhibitors for non-oncology indications remains largely un-explored. The results described here (e.g., under the Examples section) show that pharmacologic or genetic inhibition of SIK2/SIK3 ameliorates epithelial cell injury in complementary mouse and human CKD models.

In particular, these results—that are described in Example 1 of the present disclosure-demonstrated that SIK2/SIK3 inhibitors bear potential to treat chronic kidney disease. Use of orthogonal genetic and pharmacologic approaches builds confidence in the described model. SIK2/SIK3 inhibition preserves proximal tubule epithelial integrity and reduces renal fibrosis. At this point, further studies are needed to determine whether direct anti-fibrotic effects of SIK2/SIK3 inhibitors might underly the therapeutic effects seen here. Since injured epithelial cells promote fibroblast activation in vivo, it is possible SK-124 reduces fibrosis in intact kidneys due to preserved epithelial cell integrity. Irrespective of the mechanism of kidney protection, these results support cellular and molecular roles of SIK2/SIK3 in chronic kidney disease. Furthermore, these data underscore the therapeutic potential of small molecule SIK2/SIK3 inhibitors in the treatment of chronic kidney disease and its complications.

Accordingly, described herein are SIK inhibitors and use of the same in treating renal fibrosis and chronic kidney disease. The disclosure is based, in part, on the surprising findings by the present inventors that are described here, e.g., under the Examples section.

### SIK Inhibitors

The present disclosure provides SIK inhibitors, such as inhibitors that inhibit one or more of SIK1, SIK2, and SIK3. Accordingly, described herein are SIK1 inhibitors, SIK2 inhibitors, SIK3 inhibitors, and SIK2/SIK3 inhibitors.

In some embodiments, provided herein are inhibitors of human SIK1 (Gene ID: 150094). Exemplary sequence of human SIK1 is provided at NCBI Reference Sequence: NP_775490.2 (encoded by NM_173354.5).

In some embodiments, provided herein are inhibitors of human SIK2 (NCBI Gene ID: 23235). Exemplary sequence of human SIK2 is provided at NCBI Reference Sequence: NP_056006.1 (encoded by NM_015191.3).

In some embodiments, provided herein are inhibitors of human SIK3 (NCBI Gene ID: 23387). Exemplary sequence of human SIK3 isoform 1 is provided at NCBI Reference Sequence: NP_079440.3 (encoded by NM_025164.6). Exemplary sequence of human SIK3 isofonn 2 is provided at NCBI Reference Sequence: NP_001268677.1 (encoded by NM_001281748.3). Exemplary sequence of human SIK3 isoform 3 is provided at NCBI Reference Sequence: NP_001268678.1 (encoded by NM_001281749.3). Exemplary sequence of human SIK3 isoform 4 is provided at NCBI Reference Sequence: NP_001353615.1 (encoded by NM_001366686.3). Exemplary sequence of human SIK3 isoform 5 is provided at NCBI Reference Sequence: NP_001353616.1 (encoded by NM_001366687.1).

In some embodiments, the SIK inhibitors provided herein are selective inhibitors of one or more SIKs. For example, SIK inhibitors described here can be selective inhibitors of SIK2 over SIK1; selective inhibitors of SIK3 over SIK1; and/or selective inhibitors of SIK2 and SIK3, over SIK1. Thus, in some embodiments, the present disclosure provides selective SIK2/SIK3 inhibitors.

### Inhibitory Compounds

In some embodiments, the present disclosure provides inhibitory compounds that selectively inhibit one or more SIKs. For example, described herein are inhibitory compounds that selectively inhibit SIK2 and SIK3, over SIK (e.g., compounds that are selective SIK2/SIK3 inhibitors).

Inhibitory compounds for use in the methods of the present disclosure can include one or more of 8-(4-Methylimidazol-1-yl)-2-[5-[(3-methyloxetan-3-yl)methoxy]benzimidazol-1-yl]quinoline, or a pharmaceutically acceptable salt thereof: 2-[2-[5-[(3-Methyloxetan-3-yl)methoxy]benzimidazol-1-yl]-8-quinolyl]-2-azaspiro[3.3]heptan-6-amine, or a pharmaceutically acceptable salt thereof; 2-(5-((3-methyloxetan-3-yl)methoxy)-1H-benzo[d]imidazol-1-yl)-8-(1,7-diazaspiro[3.5]nonan-7-yl)quinoline, or a pharmaceutically acceptable salt thereof, 2-(5-((3-methyloxetan-3-yl)methoxy)-1H-benzo[d]imidazol-1-yl)-8-(2,7-diazaspiro[4.4]nonan-2-yl)quinoline, or a pharmaceutically acceptable salt thereof: 1-(2-(5-((3-methyloxetan-3-yl)methoxy)-1H-benzo[d]imidazol-1-yl)quinolin-8-yl)azetidin-3-anine, or a pharmaceutically acceptable salt thereof, (1-(2-(5-((3-methyloxetan-3-yl)methoxy)-1H-benzo[d]imidazol-1-yl)quinolin-8-yl)pyrrolidin-2-yl)methanamine, or a pharmaceutically acceptable salt thereof, 8-(azetidin-3-yloxy)-2-(5-((3-methyloxetan-3-yl)methoxy)-1H-benzo[d]imidazol-1-yl)quinoline, or a pharmaceutically acceptable salt thereof; 1-Methyl-9-[2-[5-[(3-methyloxetan-3-yl)methoxy]benzimidazol-1-yl]-8-quinolyl]-4-oxa-1,9-diazaspiro[5.5]undecane, or a 15 pharmaceutically acceptable salt thereof, 8-((1-Methylazetidin-3-yl)oxy)-2-(5-((3-methyloxetan-3-yl)methoxy)-1H-benzo[d]imidazol-1-yl)quinoline, or a pharmaceutically acceptable salt thereof; 8-[1-(2-Methoxyethyl)-1,7-diazaspiro[3.5]nonan-7-yl]-2-[5-[(3-methyloxetan-3-yl)methoxy]benzimidazol-1-yl]quinoline, or a pharmaceutically acceptable salt thereof, 1-(2-(7-((3-Methyloxetan-3-yl)methoxy)imidazo[1.2-a]pyridin-3-yl)quinolin-8-yl)piperidin-4-amine, or a pharmaceutically acceptable salt thereof; 3-(((1-(8-(4-Aminopiperidin-1-yl)quinoxalin-2-yl)-1H-benzo[d]imidazol-5-yl)oxy)methyl)thietane 1,1-dioxide, or a pharmaceutically acceptable salt thereof, 8-(1-Methylazetidin-3-yl)-2-(5-((3-methyloxetan-3-yl)methoxy)-1H-benzo[d]imidazol-1-yl)quinoline, or a pharmaceutically acceptable salt thereof, 2-(5-((3-Methyloxetan-3-yl)methoxy)-1H-benzo[d]imidazol-1-yl)-8-(pyrrolidin-3-yl)quinoline, or a pharmaceutically acceptable salt thereof, 2-(5-((3-Methyloxetan-3-yl)methoxy)-1H-benzo[d]imidazol-1-yl)-8-(1-methylpyrrolidin-3-yl)quinoline, or a pharmaceutically acceptable salt thereof, 8-(1-Methyl-1H-imidazol-4-yl)-2-(5-((3-methyloxetan-3-yl)methoxy)-1H-benzo[d]imidazol-1-yl)quinoline, or a pharmaceutically acceptable salt thereof, 3-(4-cyanophenyl)-N-[(6-methyl-3-pyridyl)methyl]imidazo[1,2-a]pyridine-7-carboxanide, or a pharmaceutically acceptable salt thereof, 3-(4-cyanophenyl)-N-(pyridazin-4-ylmethyl)imidazo[1,2-a]pyridine-7-carboxamide, or a pharmaceutically acceptable salt thereof; 3-(4-cyanophenyl)-N-(pyrazin-2-ylmethyl)imidazo[1,2-a]pyridine-7-carboxamide, or a pharmaceutically acceptable salt thereof; N-((1H-benzo[d]imidazol-2-yl)methyl)-3-(4-cyanophenyl)imidazo[1,2-a]pyridine-7-carboxamide, or a pharmaceutically acceptable salt thereof; 3-(4-cyanophenyl)-N-((1-methyl-1H-benzo[d]imidazol-2-yl)methyl)imidazo[1,2-a]pyridine-7-carboxanide, or a pharmaceutically acceptable salt thereof: 3-(4-cyanophenyl)-N-(imidazo[1,2-a]pyridin-2-ylmethyl)imidazo[1.2-a]pyridine-7-carboxamide, or a pharmaceutically acceptable salt thereof; N-((3H-imidazo[4,5-c]pyridin-2-yl)methyl)-3-(4-cyanophenyl)imidazo[1,2-a]pyridine-7-carboxamide, or a pharmaceutically acceptable salt thereof; N-(benzo[d]oxazol-2-ylmethyl)-3-(4-cyanophenyl)imidazo[1,2-a]pyridine-7-carboxamide, or a pharmaceutically acceptable salt thereof; 3-(4-cyanophenyl)-N-(furan-2-ylmethyl)imidazo[1,2-a]pyridine-7-carboxamide, or a pharmaceutically acceptable salt thereof; 4-(7-(1,2,3,4-tetrahydrobenzo[4,5]imidazo[1,2-a]pyrazine-2-carbonyl)imidazo[1,2-a]pyridin-3-yl)benzonitrile, or a pharmaceutically acceptable salt thereof; 3-(4-Cyanophenyl) -N-methyl-N-[(1-methylbenzimidazol-2-yl) methyl]imidazo [1,2-a]pyridine-7-carboxamide, or a pharmaceutically acceptable salt thereof; 3-(4-Cyanophenyl)-N-methyl-N-((6-methylpyridin-3-yl)methyl)imidazo[1,2-a]pyridine-7-carboxamide, or a pharmaceutically acceptable salt thereof; 3-(2,5-dimethoxyphenyl)-N-(3-pyridylmethyl)imidazo[1,2-a]pyridine-7-carboxamide, or a pharmaceutically acceptable salt thereof; 1-(4-cyanophenyl)-N-(3-pyridylmethyl)benzimidazole-5-carboxamide, or a pharmaceutically acceptable salt thereof; 1-(4-cyanophenyl)-N-(imidazo[1,2-a]pyridin-2-ylmethyl)-1H-benzo[d]imidazole-5-carboxamide, or a pharmaceutically acceptable salt thereof; N-((1H-benzo[d]imidazol-2-yl)methyl)-1-(4-cyanophenyl)-1H-benzo[d]imidazole-5-carboxamide, or a pharmaceutically acceptable salt thereof; 3-(4-Cyanophenyl)-N-(3H-imidazo[4,5-b]pyridin-2-ylmethyl)pyrazolo[1,5-a]pyridine-6-carboxamide, or a pharmaceutically acceptable salt thereof; 3-(4-cyanophenyl)-N-(3-pyridylmethyl)imidazo[1,2-a]pyrimidine-7-carboxamide, or a pharmaceutically acceptable salt thereof; 3-(4-Cyanophenyl)-N-(3-pyridylmethyl)pyrazolo[1,5-a]pyridine-6-carboxamide, or a pharmaceutically acceptable salt thereof; 4-[7-[(3-Methyloxetan-3-yl)methoxy]imidazo[1,2-a]pyridin-3-yl]benzonitrile, or a pharmaceutically acceptable salt thereof; 2-(2,6-dimethoxy-4-(7-((3-methyloxetan-3-yl)methoxy)imidazo[1,2-a]pyridin-3-yl)phenyl)-5-ethyl-1,3,4-oxadiazole, or a pharmaceutically acceptable salt thereof; 2,6-dimethoxy-4-(7-((3-methyloxetan-3-yl)methoxy)imidazo[1,2-a]pyridin-3-yl)benzonitrile, or a pharmaceutically acceptable salt thereof; 4-[7-[2-(6-Methyl-3-pyridyl)ethoxy]imidazo[1,2-a]pyridin-3-yl]benzonitrile, or a pharmaceutically acceptable salt thereof; 2-[2, 6-Dimethoxy-4-[7-[(6-methyl-3-pyridyl)methoxy]imidazo[1,2-a]pyridin-3-yl]phenyl]-5-ethyl-1,3,4-oxadiazole, or a pharmaceutically acceptable salt thereof; 4-[5-(1-Ethylpyrazol-4-yl)benzimidazo]-1-yl]benzonitrile, or a pharmaceutically acceptable salt thereof; 4-(5-(1-Cyclopropyl-1H-pyrazol-4-yl)-1H-benzo[d]imidazol-1-yl)benzonitrile, or a pharmaceutically acceptable salt thereof; 4-[7-(1-Ethylpyrazol-4-yl) imidazo[1,2-a]pyridin-3-yl]benzonitrile, or a pharmaceutically acceptable salt thereof; 5-(7-(1-ethyl-1H-pyrazol-4-yl)imidazo[1,2-a]pyridin-3-yl)picolinonitrile, or a pharmaceutically acceptable salt thereof; 4-(7-(1-ethyl-1H-pyrazol-4-yl)imidazo[1,2-a]pyridin-3-yl)-2-methylbenzonitrile, or a pharmaceutically acceptable salt thereof; 4-[7-(4-Cyclopropylimidazol-1-yl)imidazo[1,2-a]pyridin-3-yl]benzonitrile, or a pharmaceutically acceptable salt thereof; 4-(7-(1-cyclopropyl-1H-pyrazol-4-yl)imidazo[1,2-a]pyridin-3-yl)benzonitrile, or a pharmaceutically acceptable salt thereof; 4-(7-(1-(oxetan-3-yl)-1H-pyrazol-4-yl)imidazo[1,2-a]pyridin-3-yl)benzonitrile, or a pharmaceutically acceptable salt thereof; 2-[2,6-Dimethoxy-4-[7-(1-methylpyrazol-4-yl)imidazo[1,2-a]pyridin-3-yl]phenyl]-5-ethyl-1,3,4-oxadiazole, or a pharmaceutically acceptable salt thereof: 4-[6-(1-Ethylpyrazol-4-yl)pyrazolo[1,5-a]pyridin-3-yl]benzonitrile, or a pharmaceutically acceptable salt thereof; 2-(4-(6-(1-cyclopropyl-1H-pyrazol-4-yl)pyrazolo[1,5-a]pyridin-3-yl)-2,6-dimethoxyphenyl)-5-ethyl-1.3.4-oxadiazole, or a pharmaceutically acceptable salt thereof; 4-(6-(1-cyclopropyl-1H-pyrazol-4-yl)pyrazolo[1,5-a]pyridin-3-yl)benzonitrile, or a pharmaceutically acceptable salt thereof; 2-[4-[7-(1-Cyclopropylpyrazol-4-yl)imidazo[1,2-a]pyridin-3-yl]-2,6-dimethoxy-phenyl]-5-ethyl-1,3,4-oxadiazole, or a pharmaceutically acceptable salt thereof; 4-(7-(1-cyclopropyl-1H-pyrazol-4-yl)imidazo[1.2-a]pyridin-3-yl)-2,6-dimethoxybenzohydrazide, or a pharmaceutically 30 acceptable salt thereof; 2-(4-(7-(1-ethyl-1H-pyrazol-4-yl)imidazo[1,2-a]pyridin-3-yl)phenyl)-5-methyl-1,3,4-oxadiazole, or a pharmaceutically acceptable salt thereof; 2-ethyl-5-(2-methoxy-4-(7-(1-methyl-1H-pyrazol-4-yl)imidazo[1,2-a]pyridin-3-yl)phenyl)-1,3,4-oxadiazole, or a pharmaceutically acceptable salt thereof; 2-(2,6-difluoro-4-(7-(1-methyl-1H-pyrazol-4-yl)imidazo[1,2-a]pyridin-3-yl)phenyl)-5-ethyl-1.3.4-oxadiazole, or a pharmaceutically acceptable salt thereof; 2-(2,6-dichloro-4-(7-(1-methyl-1H-pyrazol-4-yl)imidazo[1,2-a]pyridin-3-yl)phenyl)-5-ethyl-1,3,4-oxadiazole, or a pharmaceutically acceptable salt thereof; 2-cyclopropyl-5-(2,6-dimethoxy-4-(7-(1-methyl-1H-pyrazol-4-yl)imidazo[1,2-a]pyridin-3-yl)phenyl)-1,3,4-oxadiazole, or a pharmaceutically acceptable salt thereof; 3-[2,6-Dimethoxy-4-[7-(1-methylpyrazol-4-yl)imidazo[1, 2-a]pyridin-3-yl]phenyl]-5-ethyl-1,2,4-oxadiazole, or a pharmaceutically acceptable salt thereof; 3-(4-(7-(1-Ethyl-1H-pyrazol-4-yl)imidazo[1,2-a]pyridin-3-yl)phenyl)-5-methyl-1,2,4-oxadiazole, or a pharmaceutically acceptable salt thereof; 4-[6-(1-Ethylpyrazol-4-yl)pyrazolo[1,5-a]pyrimidin-3-yl]benzonitrile, or a pharmaceutically acceptable salt thereof; 4-[6-(1-Cyclopropylpyrazol-4-yl)pyrazolo[1,5-a]pyrimidin-3-yl]benzonitrile, or a pharmaceutically acceptable salt thereof; 6-(1-Cyclopropylpyrazol-4-yl)-3-[4-(1-ethyltriazol-4-yl)-3,5-dimethoxy-phenyl]pyrazolo[1,5-a]pyridine, or a pharmaceutically acceptable salt thereof; 4-[7-(1-Cyclopropyltriazol-4-yl)imidazo[1, 2-a]pyridin-3-yl]benzonitrile, or a pharmaceutically acceptable salt thereof: 4-[7-(2-Ethyltetrazol-5-yl)imidazo[1,2-a]pyridin-3-yl]benzonitrile, or a pharmaceutically acceptable salt thereof; and 3-[3,5-Dimethoxy-4-(2,2,2-trifluoroethylcarbanoyl)phenyl]-N-[(6-methyl-3-pyridyl)methyl]imidazo[1,2-a]pyridine-7-carboxamide, or a pharmaceutically acceptable salt thereof. Detailed description of such inhibitors is provided, e.g., in WO 2022/031928, which is incorporated herein by reference in its entirety.

In some embodiments, the present disclosure provides compounds of Formula I:

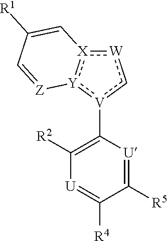

or a pharmaceutically acceptable salt thereof, wherein:
V, W, X, Y, and Z are each independently C or N;
wherein at least two of V, W, X, Y, and Z are N, and the ring comprising V, W, X, Y, and Z forms a heteroaryl ring;
U is CR³ or N;
U′ is CR⁵ or N;
U″ is CR⁶ or N;
R¹ is selected from C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, OR^{a1}, C(O)R^{b1}, C(O)NRN^{c1}R^{d1}, NR^{c1}C(O)R^{b1}, C(O)OR^{a1}, OC(O)R^{b1}, OC(O)NR^{c1}R^{d1}, and NR^{c1}C(O)OR^{a1}, wherein the C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl—are each optionally substituted with 1, 2, 3, or 4 independently selected R^{1A} substituents;
R^{a1}, R^{b1}, R^{c1}, and R^{d1} are each independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 4-10 membered heterocycloalkyl, 5-10 membered heteroaryl, C₆₋₁₀ aryl-C₁₋₆ alkyl-, C₃₋₁₀ cycloalkyl-C₁₋₆ alkyl-, (4-10 membered heterocycloalkyl)-C₁₋₆ alkyl, and (5-10 membered heteroaryl)-C₁₋₆ alkyl, wherein the C₁₋₆ alkyl, C₂₋₆ alkenyl, C₂₋₆ alkynyl, C₆₋₁₀ aryl, C₃₋₁₀ cycloalkyl. 4-10 membered heterocycloalkyl, 5-10 membered heteroaryl, C₆₋₁₀ aryl-C₁₋₆ alkyl-, C₃₋₁₀ cycloalkyl-C₁₋₆alkyl-, (4-10 membered heterocycloalkyl)-C₁₋₆ alkyl, and (5-10 membered heteroaryl)-C₁₋₆ alkyl are each optionally substituted with 1, 2, 3, or 4 independently selected R^{1A} substituents;
or R^{c1} and R^{d1}, together with the nitrogen to which they are attached, come together to form a 10-14 membered heterocycloalkyl or 10-14 membered heteroaryl group, wherein the 10-14 membered heterocycloalkyl and 10-14 membered heteroaryl group are each optionally substituted with 1, 2, 3, or 4 independently selected R^{1A} substituents;
each R^{1A} is independently selected from halo, C₁₋₆ alkyl, C₂₋₆ alkenyl, C₂₋₆ alkynyl, C₁₋₆ haloalkyl, C₁₋₆ alkoxy, C₆₋₁₀ aryl, C₃₋₁₀ cycloalkyl, 4-10 membered heterocycloalkyl, 5-10 membered heteroaryl, NO₂, CN, wherein the C₁₋₆ alkyl, C₂₋₆ alkenyl, and C₂₋₆ alkynyl are each optionally substituted with C₁₋₄ alkoxy;
R² is selected from H, halo, C₁₋₆ alkyl, C₂₋₆ alkenyl, C₂₋₆ alkynyl, C₁₋₆ haloalkyl, and C₁₋₆ alkoxy;
R³ is selected from H, halo, C₁₋₆ alkyl, C₂₋₆ alkenyl, C₂₋₆ alkynyl, C₁₋₆ haloalkyl, and C₁₋₆ alkoxy:
R⁴ is selected from H, halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 4-10 membered heterocycloalkyl, 5-10 membered heteroaryl, C₆₋₁₀ aryl-C₁₋₆ alkyl-, C₃₋₁₀ cycloalkyl-C₁₋₆ alkyl-, (4-10 membered heterocycloalkyl)-C₁₋₆ alkyl, (5-10 membered heteroaryl)-C₁₋₆ alkyl. NO₂. CN, OR^{a4}, C(O)R^{b4}, C(O)NR^{c4}R^{d4}, NR^{c4}C(O)R^{b4}, C(O)OR^{a4}, OC(O)R^{b4}, OC(O)NR^{c4}R^{d4}, and NR^{c4}C(O)OR^{a4}, wherein the C₆₋₁₀ aryl, C₃₋₁₀ cycloalkyl, 4-10 membered heterocycloalkyl, 5-10 membered heteroaryl, C₆₋₁₀ aryl-C₁₋₆ alkyl-, C₃₋₁₀ cycloalkyl-C₁₋₆ alkyl-, (4-10 membered heterocycloalkyl)-C₁₋₆ alkyl, and (5-10 membered heteroaryl)-C₁₋₆ alkyl are each optionally substituted with 1, 2, 3, or 4 independently selected R⁴¹ substituents;
R^{a4}, R^{b4}, R^{c4}, and R^{d4} are each independently selected from H, C₁₋₆ alkyl, C₂₋₆ alkenyl, C₂₋₆ alkynyl, C₁₋₆ haloalkyl, and C₁₋₆ alkoxy;
each R^{4A} is independently selected from C₁₋₆ alkyl, C₂₋₆ alkenyl, C₂₋₆ alkynyl, C₁₋₆ haloalkyl, and C₁₋₆ alkoxy:
R^{c} is selected from H, halo, C₁₋₆ alkyl, C₂₋₆ alkenyl, C₂₋₆ alkynyl, C₁₋₆ haloalkyl, and C₁₋₆ alkoxy;
or R⁴ and R⁵, together with the carbon atoms to which they are attached, come together to form a 5-6 membered aryl ring which is optionally substituted with 1, 2, 3, or 4 independently selected R⁷ substituents;
R⁶ is selected from H, C₁₋₆ alkyl, C₂₋₆ alkenyl, C₂₋₆ alkynyl, C₁₋₆ haloalkyl, and 5 C₁₋₆ alkoxy;
each R⁷ is independently selected from C₆₋₁₀ aryl, C₃₋₁₀ cycloalkyl. 4-14 membered heterocycloalkyl. 5-10 membered heteroaryl, C₆₋₁₀ aryl-C₁₋₆ alkyl-, C₃₋₁₀ cycloalkyl-C₁₋₆ alkyl-, (4-10 membered heterocycloalkyl)-C₁₋₆ alkyl, (5-10 membered heteroaryl)-C₁₋₆ alkyl, NO₂, CN, and OR^{a7}, wherein the C₆₋₁₀ aryl, C₃₋₁₀ cycloalkyl, 4-14 membered heterocycloalkyl, 5-10 membered heteroaryl, C₆₋₁₀ aryl-C₁₋₆ alkyl-, C₃₋₁₀ cycloalkyl-C₁₋₆ alkyl-, (4-10 membered heterocycloalkyl)-C₁₋₆ alkyl, (5-10 membered heteroaryl)-C₁₋₆ alkyl are each optionally substituted with 1, 2, 3, or 4 independently selected R⁷A substituents;
each R^{a7} is independently selected from C₆₋₁₀ aryl, C₃₋₁₀ cycloalkyl, 4-10 membered heterocycloalkyl, 5-10 membered heteroaryl, C₆₋₁₀ aryl-C₁₋₆ alkyl-, C₃₋₁₀ cycloalkyl-C₁₋₆ alkyl-, (4-10 membered heterocycloalkyl)-C₁₋₆ alkyl, and (5-10 membered heteroaryl)-C₁₋₆ alkyl, and
each R^{7A} is independently selected from C₁₋₆ alkyl, C₂₋₆ alkenyl, C₂₋₆ alkynyl, C₁₋₆ haloalkyl, C₁₋₆ alkoxy, amino, C₁₋₆ alkylamino, di(C₁₋₆ alkyl)amino, phenyl, C₃₋₆ cycloalkyl, 4-6 membered heterocycloalkyl, 5-6 membered heteroaryl, and wherein the C₁₋₆ alkyl, C₂₋₆ alkenyl, and C₂₋₆ alkynyl are each optionally substituted with amino or C₁₋₄ alkoxy.

In some embodiments, two of V, W, X, Y, and Z are N.

In some embodiments, three of V, W, X. Y, and Z are N.

In some embodiments, four of V, W, X, Y, and Z are N.

In some embodiments, V, X, and Z are each C, and W and Y are each N.

In some embodiments, X, Y, and Z are each C, and V and W are each N.

In some embodiments, V, Y, and Z are each C, and W and X are each N.

In some embodiments, V and Y are each C, and W, X, and Z are each N.

In some embodiments, R¹ is selected from phenyl, C₃₋₆ cycloalkyl, 5-6 membered heteroaryl, 4-6 membered heterocycloalkyl, OR^{a1}, C(O)NR^{c1}R^{dl}, and NR^{c1}C(O)R^{b1}, C(O)OR^{a1}, OC(O)R^{b1}, OC(O)NR^{c1}R^{d1}, and NR^{c1}C(O)OR^{a1}, wherein the phenyl, C₃₋₆ cycloalkyl. 5-10 membered heteroaryl, and 4-6 membered heterocycloalkyl, are each optionally substituted with 1, 2, 3, or 4 independently selected R^{1A} substituents.

In some embodiments, R¹ is selected from 5-10 membered heteroaryl, OR^{a1} C(O)NR^{c1}R^{d1}, and NR^{c1}C(O)R^{b1}, wherein the 5-10 membered heteroaryl is optionally substituted with 1, 2, 3, or 4 independently selected RA substituents.

In some embodiments, R¹ is selected from 5-6 membered heteroaryl, OR^{a1}, C(O)NR^{c1}R^{d1}, and NR^{c1}C(O)R^{b1}, wherein the 5-6 membered heteroaryl is optionally substituted with 1, 2, 3, or 4 independently selected RA “substituents.

In some embodiments, R¹ is selected from 5-6 membered heteroaryl, OR^{a1}, C(O)NR^{b1}R^{d1}, and NR^{c1}C(O)R^{b1}, wherein the 5-6 membered heteroaryl is optionally substituted with 1 or 2 independently selected R^{1A} substituents.

In some embodiments, R^{a1}, R^{b1}, R^{c1}, and R^{d1} are each independently selected from H, 4-10 membered heterocycloalkyl, (4-10 membered heterocycloalkyl)-C₁₋₆ alkyl, and (5-10 membered heteroaryl)-C₁₋₆ alkyl, wherein the 4-10 membered heterocycloalkyl. (4-10 membered heterocycloalkyl)-C₁₋₆ alkyl, and (5-10 membered heteroaryl)-C₁₋₆ alkyl are each optionally substituted with 1 or 2 independently selected R^{1A} substituents:
or R^{c1} and R^{d1}, together with the nitrogen to which they are attached, come together to form a 10-14 membered heterocycloalkyl or 10-14 membered heteroaryl group.

In some embodiments, R^{a1}, R^{b1}, R^{c1}, and R^{d1} are each independently selected from H, 4-10 membered heterocycloalkyl, (4-10 membered heterocycloalkyl)-C₁₋₆ alkyl, and (5-10 membered heteroaryl)-C₁₋₆ alkyl, wherein the 4-10 membered heterocycloalkyl. (4-10 membered heterocycloalkyl)-C₁₋₆ alkyl, and (5-10 membered heteroaryl)-C₁₋₆ alkyl are each optionally substituted with 1, 2, 3, or 4 independently selected R^{1A} substituents.

In some embodiments, R^{a1}, R^{b1}, R^{c1}, and R^{d1} are each independently selected from H, 4-10 membered heterocycloalkyl, (4-10 membered heterocycloalkyl)-C₁₋₆ alkyl, and (5-10 membered heteroaryl)-C₁₋₆ alkyl, wherein the 4-10 membered heterocycloalkyl, (4-10 membered heterocycloalkyl)-C₁₋₆ alkyl, and (5-10 membered heteroaryl)-C₁₋₆ alkyl are each optionally substituted with 1 or 2 independently selected Ria substituents.

In some embodiments, R^{c1} and R^{d1} together with the nitrogen to which they are attached, come together to form a 10-14 membered heterocycloalkyl or 10-14 membered heteroaryl group.

In some embodiments, R^{c1} and R^{d1} together with the nitrogen to which they are attached, come together to form a 10-14 membered heterocycloalkyl group.

In some embodiments, R^{c1} and R^{d1} together with the nitrogen to which they are attached, come together to form a 10-14 membered heteroaryl group.

In some embodiments, R^{a1}, R^{b1}, R^{c1}, and R^{d1} are each independently selected from H, methyl, pyrazolyl, pyridinylmethyl, pyridinylethyl, imidazo[1,2-a]pyridinylmethyl, benzoimidazolylmethyl, imidazo[4,5-c]pyridinylmethyl, benzoxazolylmethyl, oxetanylmethyl, oxetanylethyl, thietanyl-(1.1-dioxide)methyl, 2-oxaspiro[3.3]heptanyl, and 2-oxaspiro[3.5]nonanyl, wherein the methyl, pyrazolyl, pyridinylmethyl, pyridinylethyl, imidazo[1,2-a]pyridinylmethyl, benzoimidazolylmethyl, imidazo[4,5-c]pyridinylmethyl, benzoxazolylmethyl, oxetanylmethyl, oxetanylethyl, thietanyl-(1,1-dioxide)methyl. 2-oxaspiro[3.3]heptanyl, and 2-oxaspiro[3.5]nonanyl are each optionally substituted with 1 or 2 independently selected R^{1A} substituents;
or R^{c1} and R^{d1}, together with the nitrogen to which they are attached, come together to form 1,2,3,4-tetrahydrobenzo[4,5]imidazo[1,2-a]pyrazinyl.

In some embodiments, each R^{1A} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, and CN, wherein the C₁₋₆ alkyl, C₂₋₆ alkenyl, and C₂₋₆ alkynyl are each optionally substituted with C₁₋₄ alkoxy.

In some embodiments, each R^{1A} is independently selected from C₁₋₆ alkyl, C₁₋₆ haloalkyl, C₃₋₁₀ cycloalkyl, 4-10 membered heterocycloalkyl and CN, wherein the C₁₋₆ alkyl is optionally substituted with C₁₋₄ alkoxy.

In some embodiments, each R^{1A} is independently selected from C₁₋₆ alkyl, C₁₋₆ haloalkyl, C₃₋₆ cycloalkyl, 4-6 membered heterocycloalkyl, and CN, wherein the C₁₋₆ alkyl is optionally substituted with C₁₋₄ alkoxy.

In some embodiments, each R^{1A} is independently selected from C₁₋₆ alkyl and CN, wherein each C₁₋₆ alkyl is optionally substituted with C₁₋₄ alkoxy.

In some embodiments, each R^{1A} is independently selected from C₁₋₄ alkyl, C₃₋₆ cycloalkyl, 4-6 membered heterocycloalkyl, and CN, wherein each C₁₋₄ alkyl is optionally substituted with methoxy.

In some embodiments, each RA is independently selected from C₁₋₄ alkyl and CN, wherein each C₁₋₄ alkyl is optionally substituted with methoxy.

In some embodiments, each R^{1A} is independently selected from methyl, ethyl, methoxymethyl, cyclopropyl, oxetanyl, and CN.

In some embodiments, R² is selected from H, C₁₋₆ alkyl, C₁₋₆ haloalkyl, and C₁₋₆ alkoxy.

In some embodiments, R² is selected from H, C₁₋₆ alkyl, and C₁₋₆ alkoxy.

In some embodiments, R² is selected from H and C₁₋₆ alkoxy.

In some embodiments, R² is selected from H and C₁₋₄ alkoxy.

In some embodiments, R² is selected from H and methoxy.

In some embodiments, R² is H.

In some embodiments, R² is methoxy.

In some embodiments, U is CR³.

In some embodiments, R³ is selected from H, halo, C₁₋₆ alkyl, C₁₋₆ haloalkyl, and C₁₋₆ alkoxy.

In some embodiments, R³ is selected from H, halo, C₁₋₆ alkyl, and C₁₋₆ alkoxy.

In some embodiments, R³ is selected from H, halo, and C₁₋₆ alkoxy.

In some embodiments, R³ is selected from H, halo, and C₁₋₄ alkoxy.

In some embodiments, R³ is selected from H, fluoro, chloro, and methoxy.

In some embodiments, R³ is H.

In some embodiments, R³ fluoro.

In some embodiments, R³ is chloro.

In some embodiments, R³ is methoxy.

In some embodiments, U is N.

In some embodiments, U″ is CR⁶.

In some embodiments, R⁶ is selected from H, C₁₋₆ alkyl, and C₁₋₆ alkoxy.

In some embodiments, R⁶ is selected from H and C₁₋₆ alkoxy.

In some embodiments, R⁶ is selected from H and C₁₋₄ alkoxy.

In some embodiments, R⁶ is selected from H and methoxy.

In some embodiments, R⁶ is H.

In some embodiments, U″ is N.

In some embodiments, U is CR³ and U′ is CR⁶.

In some embodiments, U is N and U″ is CR⁶.

In some embodiments, U is CR³ and U″ is N.

In some embodiments, U is N and U″ is N.

In some embodiments, R⁴ is selected from H, C₆₋₁₀ aryl, C₃₋₁₀ cycloalkyl, 4-10 membered heterocycloalkyl. 5-10 membered heteroaryl, CN, OR^{a4}, C(O)R^{b4}. C(O)NR^{c4}R^{d4}, NR^{c4}C(O)R^{b4}, C(O)OR^{a4}, OC(O)R^{b4}, OC(O)NR^{c4}R^{d4}, and NR^{c4}C(O)OR^{a4}, wherein the C₆₋₁₀ aryl, C₃₋₁₀ cycloalkyl, 4-10 membered heterocycloalkyl, and 5-10 membered heteroaryl are each optionally substituted with 1, 2, 3, or 4 independently selected R⁴ substituents.

In some embodiments, R⁴ is selected from H, phenyl, C₃₋₆ cycloalkyl, 4-6 membered heterocycloalkyl, 5-6 membered heteroaryl, CN, and C(O)NR^{c4}R^{d4}, wherein the phenyl, C₃₋₆ cycloalkyl, 4-6 membered heterocycloalkyl, and 5-6 membered heteroaryl are each optionally substituted with 1 or 2 independently selected R⁴, substituents.

In some embodiments, R⁴ is selected from H, 5-10 membered heteroaryl, CN, and C(O)NR⁴R^{d4}, wherein the 5-10 membered heteroaryl is optionally substituted with 1, 2, 3, or 4 independently selected R^{4A} substituents.

In some embodiments, R⁴ is selected from H, 5-6 membered heteroaryl. CN, and C(O)NR^{c4}R^{d4}, wherein the 5-6 membered heteroaryl is optionally substituted with 1, 2, 3, or 4 independently selected R^{4A} substituents.

In some embodiments, R⁴ is selected from H, 5-6 membered heteroaryl, CN, C(O)NR^{c4}R^{d4}, and C(O)N(R^{c4})NR^{c4}R^{d4}, wherein the 5-6 membered heteroaryl is optionally substituted with 1 or 2 independently selected R^{4A} substituents.

In some embodiments, R⁴ is selected from H, 5-6 membered heteroaryl, CN, and C(O)NR⁴R^{d4}, wherein the 5-6 membered heteroaryl is optionally substituted with 1 or 2 independently selected R^{4A} substituents.

In some embodiments, R⁴ is C(O)N(R^{c4})NR^{c4}R^{d4}.

In some embodiments, R^{a4}, R^{b4}. R^{c4}, and R^{d4} are each independently selected from H, C₁₋₆ alkyl, and C₁₋₆ haloalkyl.

In some embodiments, R^{c4} and R^{d4} are each independently selected from H, C₁₋₆ alkyl, and C₁₋₆ haloalkyl.

In some embodiments, R^{a4}, R^{b4}, R^{c4}, and R^{d4} are each independently selected from H, C₁₋₄ alkyl, and C₁₋₄ haloalkyl.

In some embodiments, R^{c4} and R^{d4} are each independently selected from H, C₁₋₄ alkyl, and C₁₋₄ haloalkyl.

In some embodiments, R^{a4}, R^{b4}, R^{c4}, and R^{d4} are each independently selected from H, ethyl, and trifluoroethyl.

In some embodiments, R^{c4} and R^{d4} are each independently selected from H, ethyl, and trifluoroethyl.

In some embodiments, R⁴ is selected from H, CN, oxadiazolyl, and C(O)NR⁴R^{d4}, wherein the oxadiazolyl is optionally substituted with 1, 2, 3, or 4 independently selected R⁴ substituents; and R^{c4} and R^{d4} are each independently selected from H, C₁₋₄ alkyl, and C₁₋₄ haloalkyl.

In some embodiments, R⁴ is selected from H, CN, oxadiazolyl, and C(O)NR^{c4}R^{d4}, wherein the oxadiazolyl is optionally substituted with 1 or 2 independently selected R^{4A} substituents; and R^{c4} and R^{d4} are each independently selected from H, C₁₋₄ alkyl, and C₁₋₄ haloalkyl.

In some embodiments, R⁴ is selected from H, CN, oxadiazolyl, and C(O)NHCH₂CF₃, wherein the oxadiazolyl is optionally substituted with 1, 2, 3, or 4 independently selected R^{4A} substituents.

In some embodiments, R⁴ is selected from H, CN, oxadiazolyl, and C(O)NHCH₂CF₃, wherein the oxadiazolyl is optionally substituted with 1 or 2 independently selected R^{4A} substituents.

In some embodiments, each R^{4A} is independently selected from C₁₋₆ alkyl, C₁₋₆ haloalkyl, and C₁₋₆ alkoxy.

In some embodiments, each R^{4A} is independently selected from C₁₋₆ alkyl and C₁₋₆ haloalkyl.

In some embodiments, each R^{4A} is independently selected from C₁₋₄ alkyl and C₁₋₄ haloalkyl.

In some embodiments, each R^{4A} is an independently selected C₁₋₆ alkyl group.

In some embodiments, each R^{4A} is an independently selected C₁₋₆ haloalkyl group.

In some embodiments, each R^{4A} is an independently selected C₁₋₄ alkyl group.

In some embodiments, each R^{4A} is an independently selected C₁₋₄ haloalkyl group.

In some embodiments, each R^{4A} is ethyl.

In some embodiments, U′ is N.

In some embodiments, U′ is CR⁵.

In some embodiments, one of U, U′, and U″ is N.

In some embodiments, two of U, U′, and U″ are N.

In some embodiments, R⁵ is selected from H, halo, C₁₋₆ alkyl, C₁₋₆ haloalkyl, and C₁₋₆ alkoxy.

In some embodiments, R⁵ is selected from H, halo, C₁₋₆ alkyl, and C₁₋₆ alkoxy.

In some embodiments, R⁵ is selected from H, halo, and C₁₋₆ alkoxy.

In some embodiments, R⁵ is selected from H, halo, C₁₋₄ alkyl, and C₁₋₄ alkoxy.

In some embodiments, R⁵ is selected from H, halo, and C₁₋₄ alkoxy.

In some embodiments, R⁵ is selected from H, fluoro, chloro, and methoxy.

In some embodiments, R⁵ is H.

In some embodiments, R⁵ is fluoro.

In some embodiments, R⁵ is chloro.

In some embodiments, R⁵ is methoxy.

In some embodiments, R³ and R⁵ are each an independently selected halo.

In some embodiments, R³ and R⁵ are each fluoro. In some embodiments, R³ and R⁵ are each chloro.

In some embodiments, R³ and R⁵ are each an independently selected C₁₋₆ alkoxy.

In some embodiments, R³ and R⁵ are each methoxy.

In some embodiments, R⁴ and R⁵ together with the carbon atoms to which they are attached, come together to form a 5-6 membered aryl ring which is optionally substituted with 1, 2, 3, or 4 independently selected R⁷ substituents.

In some embodiments, R⁴ and R⁵ together with the carbon atoms to which they are attached, come together to form a 5-6 membered aryl ring which is optionally substituted with 1 or 2 independently selected R⁷ substituents.

In some embodiments, R⁴ and R⁵ together with the carbon atoms to which they are attached, come together to form a 5-membered aryl ring which is optionally substituted with 1 or 2 independently selected R⁷ substituents.

In some embodiments, R⁴ and R⁵ together with the carbon atoms to which they are attached, come together to form a 6-membered aryl ring which is optionally substituted with 1 or 2 independently selected R⁷ substituents.

In some embodiments, each R⁷ is independently selected from phenyl, C₃₋₁₀ 5 cycloalkyl, 4-14 membered heterocycloalkyl, 5-10 membered heteroaryl, phenyl-C₁₋₆ alkyl-, C₃₋₁₀ cycloalkyl-C₁₋₆ alkyl-, (4-10 membered heterocycloalkyl)-C₁₋₆ alkyl, (5-10 membered heteroaryl)-C₁₋₆ alkyl, and OR^{a7}, wherein the phenyl, C₃₋₁₀ cycloalkyl, 4-14 membered heterocycloalkyl, 5-10 membered heteroaryl, phenyl-C₁₋₆ alkyl-, C₃₋₁₀ cycloalkyl-C₁₋₆ alkyl-, (4-10 membered heterocycloalkyl)-C₁₋₆ alkyl, (5-10 membered 10 heteroaryl)-C₁₋₆ alkyl are each optionally substituted with 1, 2, 3, or 4 independently selected R^{7A} substituents.

In some embodiments, each R⁷ is independently selected from phenyl. C₃₋₁₀ cycloalkyl, 4-14 membered heterocycloalkyl, 5-10 membered heteroaryl, phenyl-C₁₋₆ alkyl-, C₃₋₁₀ cycloalkyl-C₁₋₆ alkyl-, (4-10 membered heterocycloalkyl)-C₁₋₆ alkyl, (5-10 membered heteroaryl)-C₁₋₆ alkyl, and OR^{a7}, wherein the phenyl, C₃₋₁₀ cycloalkyl, 4-14 membered heterocycloalkyl, 5-10 membered heteroaryl, phenyl-C₁₋₆ alkyl-, C₃₋₁₀ cycloalkyl-C₁₋₆ alkyl-, (4-10 membered heterocycloalkyl)-C₁₋₆ alkyl, (5-10 membered heteroaryl)-C₁₋₆ alkyl are each optionally substituted with 1 or 2 independently selected R^{7A} substituents.

In some embodiments, each R⁷ is independently selected from C₃₋₁₀ cycloalkyl, 4-14 membered heterocycloalkyl, 5-10 membered heteroaryl, and OR^{a7}, wherein the C₃₋₁₀ cycloalkyl, 4-14 membered heterocycloalkyl, and 5-10 membered heteroaryl are each optionally substituted with 1, 2, 3, or 4 independently selected R^{7A} substituents.

In some embodiments, each R⁷ is independently selected from C₃₋₁₀ cycloalkyl, 4-14 membered heterocycloalkyl, 5-10 membered heteroaryl, and OR^{a7}, wherein the C₃₋₁₀ cycloalkyl, 4-14 membered heterocycloalkyl, and 5-10 membered heteroaryl are each optionally substituted with 1 or 2 independently selected R^{7A} substituents.

In some embodiments, each R^{a7} is independently selected from phenyl, C₃₋₆ cycloalkyl, 4-6 membered heterocycloalkyl, 5-6 membered heteroaryl, phenyl-C₁₋₆ alkyl-, C₃₋₆ cycloalkyl-C₁₋₆ alkyl-, (4-6 membered heterocycloalkyl)-C₁₋₆ alkyl, and (5-6 membered heteroaryl)-C₁₋₆ alkyl.

In some embodiments, each R^{a7} is independently selected from 4-10 membered heterocycloalkyl and 5-10 membered heteroaryl.

In some embodiments, each R^{a7} is independently selected from 4-6 membered heterocycloalkyl and 5-6 membered heteroaryl.

In some embodiments, each R^{a7} is an independently selected 4-10 membered heterocycloalkyl group.

In some embodiments, each R^{a7} is an independently selected 4-6 membered heterocycloalkyl group.

In some embodiments, each R^{a7} is azetidinyl.

In some embodiments, each R⁷ is independently selected from bicyclo[1.1.1]pentanyl, azetidinyl, pyrrolidinyl, piperidinyl, morpholinyl, imidazolyl, azaspiro[3.3]heptanyl, diazaspiro[3.5]nonanyl, oxadiazaspiro[5.5]undecanyl, diazaspiro[4.4]nonanyl, and azetidinyloxy, wherein the bicyclo[1.1.1]pentanyl, azetidinyl, pyrrolidinyl, piperidinyl, morpholinyl, imidazolyl, azaspiro[3.3]heptanyl, diazaspiro[3.5]nonanyl, oxadiazaspiro[5.5]undecanyl, and diazaspiro[4.4]nonanyl are each optionally substituted with 1, 2, 3, or 4 independently selected R^{7A} substituents.

In some embodiments, each R⁷ is independently selected from bicyclo[1.1.1]pentanyl, azetidinyl, pyrrolidinyl, piperidinyl, morpholinyl, imidazolyl, azaspiro[3.3]heptanyl, diazaspiro[3.5]nonanyl, oxadiazaspiro[5.5]undecanyl, diazaspiro[4.4]nonanyl, and azetidinyloxy, wherein the bicyclo[1.1.1]pentanyl, azetidinyl, pyrrolidinyl, piperidinyl, morpholinyl, imidazolyl, azaspiro[3.3]heptanyl, diazaspiro[3.5]nonanyl, oxadiazaspiro[5.5]undecanyl, and diazaspiro[4.4]nonanyl are each optionally substituted with 1 or 2 independently selected R^{7A} substituents.

In some embodiments, each R^{7A} is independently selected from C₁₋₆ alkyl, amino, C₁₋₆ alkylamino, di(C₁₋₆ alkyl)amino, C₃₋₆ cyclopropyl, and 4-6 membered heterocycloalkyl, wherein each C₁₋₆ alkyl is optionally substituted with amino or C₁₋₄ alkoxy.

In some embodiments, each R^{7A} is independently selected from C₁₋₆ alkyl, amino, C₃₋₆ cyclopropyl and 4-6 membered heterocycloalkyl, wherein each C₁₋₆ alkyl is optionally substituted with amino or C₁₋₄ alkoxy.

In some embodiments, each R^{7A} is independently selected from C₁₋₆ alkyl, amino, C₃₋₆ cyclopropyl and 4-6 membered heterocycloalkyl, wherein each C₁₋₆ alkyl is optionally substituted with amino or methoxy.

In some embodiments, each R⁷A is independently selected from methyl, ethyl, amino, cyclopropyl, and oxetanyl, wherein each methyl and ethyl is optionally substituted with amino or methoxy.

In some embodiments, each R⁷A is independently selected from methyl, 5 methoxyethyl, aminomethyl, amino, cyclopropyl, and oxetanyl.

In some embodiments:
V, X, and Z are each C, and W and Y are each N; or
X, Y, and Z are each C, and V and W are each N; or
V, Y, and Z are each C, and W and X are each N; or
V and Y are each C, and W, X, and Z are each N;
R¹ is selected from 5-10 membered heteroaryl, OR^{a1}, C(O)NR^{b1}R^{d1}, and NR^{c1}C(O)R^{b1}, wherein the 5-10 membered heteroaryl is optionally substituted with 1, 2, 3, or 4 independently selected R^{1A} substituents;
R^{a1}, R^{b1}, R^{c1}, and R^{d1} are each independently selected from H, 4-10 membered heterocycloalkyl. (4-10 membered heterocycloalkyl)-C₁₋₆ alkyl, and (5-10 membered heteroaryl)-C₁₋₆ alkyl, wherein the 4-10 membered heterocycloalkyl, (4-10 membered heterocycloalkyl)-C₁₋₆ alkyl, and (5-10 membered heteroaryl)-C₁₋₆ alkyl are each optionally substituted with 1 or 2 independently selected R^{1A} substituents;
or R^{c1} and R^{d1}, together with the nitrogen to which they are attached, come together to form a 10-14 membered heterocycloalkyl or 10-14 membered heteroaryl group:
each RA is independently selected from C₁₋₆ alkyl, C₃₋₁₀ cycloalkyl, 4-10 membered heterocycloalkyl, and CN, wherein each C₁₋₆ alkyl is optionally substituted with C₁₋₄ alkoxy;
R² is H or C₁₋₆ alkoxy;
U is CR³ or N;
U′ is CR⁵ or N;
U″ is CR⁶ or N;
R³ is selected from H, halo, and C₁₋₆ alkoxy:
R⁴ is selected from H, 5-10 membered heteroaryl, CN, and C(O)NR^{c4}R^{d4},

wherein the 5-10 membered heteroaryl is optionally substituted with 1, 2, 3, or 4 independently selected R^{4A} substituents;
R^{c4} and R^{d4} are each independently selected from H, C₁₋₆ alkyl, and C₁₋₆ haloalkyl;
each R^{4A} is an independently selected C₁₋₆ alkyl group;
R⁵ is selected from H, halo, C₁₋₆ alkyl, and C₁₋₆ alkoxy;
or R⁴ and R⁵ together with the carbon atoms to which they are attached, come together to form a 5-6 membered aryl ring which is optionally substituted with 1, 2, 3, or 4 independently selected R⁷ substituents;
R⁶ is selected from H and C₁₋₆ alkoxy;
each R⁷ is independently selected from C₃₋₁₀ cycloalkyl, 4-14 membered heterocycloalkyl, 5-10 membered heteroaryl, and OR⁷, wherein the C₃₋₁₀ cycloalkyl, 4-14 membered heterocycloalkyl, and 5-10 membered heteroaryl are each optionally substituted with 1 or 2 independently selected R⁷A substituents;
each R^{a7} is an independently selected 4-10 membered heterocycloalkyl group; and
each R^{7A} is independently selected from C₁₋₆ alkyl, amino, C₃₋₆ cyclopropyl and 4-6 membered heterocycloalkyl, wherein each C₁₋₆ alkyl is optionally substituted with amino or C₁₋₄ alkoxy.

In some embodiments:
V, X, and Z are each C, and W and Y are each N; or
X, Y, and Z are each C, and V and W are each N; or
V. Y, and Z are each C, and W and X are each N; or
V and Y are each C, and W, X, and Z are each N;
R¹ is selected from 5-6 membered heteroaryl, OR^{a1}, C(O)NR^{b1}R^{d1}, and NR^{b1}C(O)R^{b1}, wherein the 5-6 membered heteroaryl is optionally substituted with 1 or 2 independently selected R^{1A} substituents;
R^{a1}, R^{b1}, R^{c1}, and R^{d1} are each independently selected from H, 4-10 membered heterocycloalkyl, (4-10 membered heterocycloalkyl)-C₁₋₆ alkyl, and (5-10 membered heteroaryl)-C₁₋₆ alkyl, wherein the 4-10 membered heterocycloalkyl, (4-10 membered heterocycloalkyl)-C₁₋₆ alkyl, and (5-10 membered heteroaryl)-C₁₋₆ alkyl are each optionally substituted with 1 or 2 independently selected R^{1A} substituents:
or R^{e1} and R^{d1}, together with the nitrogen to which they are attached, come together to form a 10-14 membered heterocycloalkyl or 10-14 membered heteroaryl group;
each R^{1A} is independently selected from C₁₋₆ alkyl, C₃₋₆ cycloalkyl, 4-6 membered heterocycloalkyl, and CN, wherein each C₁₋₆ alkyl is optionally substituted with C₁₋₄ alkoxy;
R² is H or C₁₋₆ alkoxy;
U is CR³ or N;
U′ is CR⁵ or N;
U″ is CR⁶ or N;
R³ is selected from H, halo, and C₁-6 alkoxy;
R⁴ is selected from H, 5-6 membered heteroaryl, CN, and C(O)NR^{c4}R^{d4}, wherein the 5-6 membered heteroaryl is optionally substituted with 1 or 2 independently selected R⁴ substituents:
R^{c4} and R^{d4} are each independently selected from H, C₁₋₆ alkyl, and C₁₋₆ haloalkyl:
each R^{4A} is an independently selected C₁₋₆ alkyl group;
R⁵ is selected from H, halo, C₁₋₆ alkyl, and C₁₋₆ alkoxy;
or R⁴ and R⁵ together with the carbon atoms to which they are attached, come together to form a 6-membered aryl ring which is optionally substituted with 1 or 2 independently selected R⁷ substituents;
each R⁷ is independently selected from C₃₋₁₀ cycloalkyl, 4-14 membered 20 heterocycloalkyl. 5-10 membered heteroaryl, and OR⁷, wherein the C₃₋₁₀ cycloalkyl, 4-14 membered heterocycloalkyl, and 5-10 membered heteroaryl are each optionally substituted with 1 or 2 independently selected R⁷A substituents;
each R⁷ is an independently selected 4-10 membered heterocycloalkyl group; and
each R^{7A} is independently selected from C₁₋₆ alkyl, amino, C₃₋₆ cyclopropyl and 4-6 membered heterocycloalkyl, wherein each C₁₋₆ alkyl is optionally substituted
with amino or C₁₋₄ alkoxy.

In some embodiments:
V, X, and Z are each C, and W and Y are each N; or
X Y, and Z are each C, and V and W are each N; or
V, Y, and Z are each C, and W and X are each N; or
V and Y are each C, and W, X, and Z are each N;
R¹ is selected from 5-6 membered heteroaryl, OR^{a1}, C(O)NR^{c1}R^{d1}, and NR^{c1}C(O)R^{b1}, wherein the 5-6 membered heteroaryl is optionally substituted with 1 or 2 independently selected R^{1A} substituents;
R^{a1}, R^{b1}, R^{c1}, and R^{d1} are each independently selected from H, methyl, pyrazolyl, pyridinylmethyl, pyridinylethyl, imidazo[1,2-a]pyridinylmethyl, benzoimidazolylmethyl, imidazo[4,5-c]pyridinylmethyl, benzoxazolylmethyl, oxetanylmethyl, oxetanylethyl, thietanyl-(1,1-dioxide)methyl. 2-oxaspiro[3.3]heptanyl, and 2-oxaspiro[3.5]nonanyl, wherein the methyl, pyrazolyl, pyridinylmethyl, pyridinylethyl, imidazo[1,2-a]pyridinylmethyl, benzoimidazolylmethyl, imidazo[4,5-c]pyridinylmethyl, benzoxazolylmethyl, oxetanylmethyl, oxetanylethyl, thietanyl-(1,1-dioxide)methyl. 2-oxaspiro[3.3]heptanyl, and 2-oxaspiro[3.5]nonanyl are each optionally substituted with 1 or 2 independently selected R^{1A} substituents;
or R^{c1} and R^{d1}, together with the nitrogen to which they are attached, come together to form 1,2,3,4-tetrahydrobenzo[4,5]imidazo[1,2-a]pyrazinyl;
each R^{1A} is independently selected from methyl, ethyl, methoxymethyl, and CN:
R² is H or C₁₋₆ alkoxy;
U is CR³ or N;
U′ is CR⁵ or N:
U″ is CR⁶ or N:
R³ is selected from H, halo, and C₁₋₆ alkoxy;
R⁴ is selected from H, 5-6 membered heteroaryl, CN, and C(O)NR^{c4}R^{d4}, wherein the 5-6 membered heteroaryl is optionally substituted with 1 or 2 independently selected R^{4A} substituents;
R^{c4} and R^{d4} are each independently selected from H, C₁₋₆ alkyl, and C₁₋₆ haloalkyl;
each R^{4A} is an independently selected C₁₋₆ alkyl group;
R⁵ is selected from H, halo, C₁₋₆ alkyl, and C₁₋₆ alkoxy;
or R⁴ and R⁵ together with the carbon atoms to which they are attached, come together to form a 6-membered aryl ring which is optionally substituted with 1 or 2 independently selected R⁷ substituents;
each R⁷ is independently selected from bicyclo[1.1.1]pentanyl, azetidinyl, pyrrolidinyl, piperidinyl, morpholinyl, imidazolyl, azaspiro[3.3]heptanyl, diazaspiro[3.5]nonanyl, oxadiazaspiro[5.5]undecanyl, diazaspiro[4.4]nonanyl, and azetidinyloxy, wherein the bicyclo[1.1.1]pentanyl, azetidinyl, pyrrolidinyl, piperidinyl, morpholinyl, imidazolyl, azaspiro[3.3]heptanyl, diazaspiro[3.5]nonanyl, oxadiazaspiro[5.5]undecanyl, and diazaspiro[4.4]nonanyl are each optionally substituted with 1 or 2 R^{7A} substituents; and
each R^{7A} is independently selected from methyl, methoxyethyl, aminomethyl, amino, cyclopropyl, and oxetanyl.

In some embodiments, the compound of Formula I is a compound of Formula II:

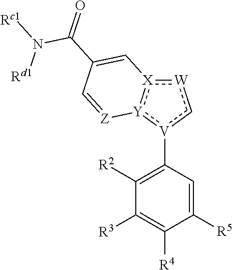

or a pharmaceutically acceptable salt thereof, wherein variables V, W, X, Y, Z, R^{c1}, R^{d1}, R², R³, R⁴, and R⁵ are defined according to the definitions provided herein for compounds of Formula I.

In some embodiments, the compound of Formula I is a compound of Formula III:

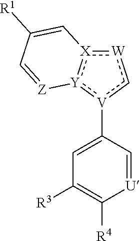

or a pharmaceutically acceptable salt thereof, wherein R¹ is a 5-10 membered heteroaryl which is optionally with 1, 2, 3, or 4 R^{1A} substituents, wherein variables U′, V, W, X, Y, Z, R^{1A}, R³, R⁴, and R⁵ are defined according to the definitions provided herein for compounds of Formula I.

In some embodiments, the compound of Formula I is a compound of Formula IIIa:

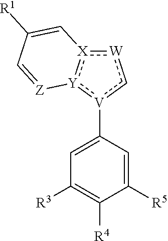

or a pharmaceutically acceptable salt thereof, wherein R¹ is a 5-10 membered heteroaryl which is optionally with 1, 2, 3, or 4 R^{1A} substituents, wherein variables V, W, X, Y, Z, R^{1A}, R³. R⁴, and R⁵ are defined according to the definitions provided herein for compounds of Formula I.

In some embodiments, the compound of Formula I is a compound of Formula IV:

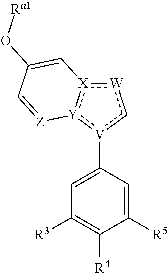

or a pharmaceutically acceptable salt thereof, wherein variables V, W, X, Y, Z, R^{a1}, R³, R⁴, and R⁵ are defined according to the definitions provided herein for compounds of Formula I.

In some embodiments, the compound of Formula I is a compound of Formula V:

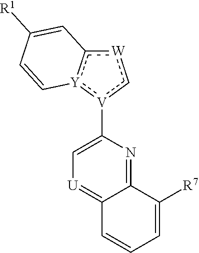

or a pharmaceutically acceptable salt thereof, wherein variables U, V. W, Y, R, and R⁷ are defined according to the definitions provided herein for compounds of Formula I.

In some embodiments, the compound provided herein (e.g., the compound of any one of Formulas I-V), or a pharmaceutically acceptable salt thereof, is selected from:

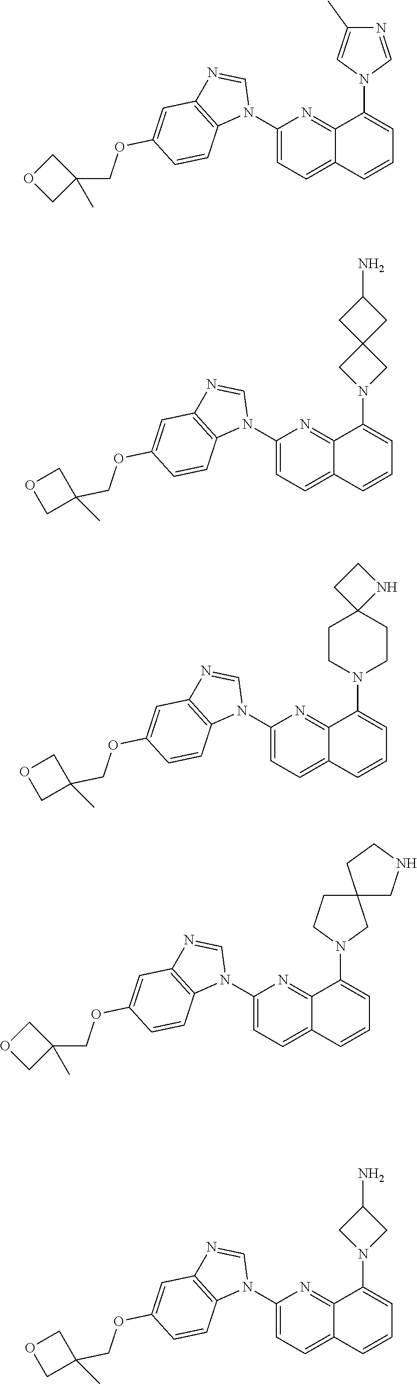 [SMILES: Cc1cn(-c2cccc3ccc(-n4cnc5cc(OCC6(C)COC6)ccc54)nc23)cn1 ; CC1(COc2ccc3c(c2)ncn3-c2ccc3cccc(N4CC5(CC(N)C5)C4)c3n2)COC1 ; CC1(COc2ccc3c(c2)ncn3-c2ccc3cccc(N4CCC5(CCN5)CC4)c3n2)COC1 ; CC1(COc2ccc3c(c2)ncn3-c2ccc3cccc(N4CCC5(CCNC5)C4)c3n2)COC1 ; CC1(COc2ccc3c(c2)ncn3-c2ccc3cccc(N4CC(N)C4)c3n2)COC1]

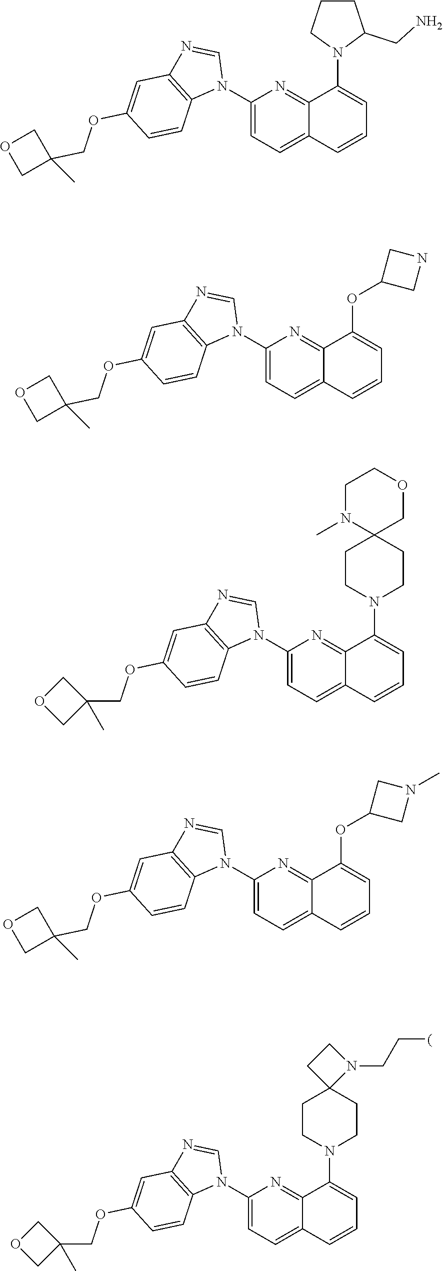

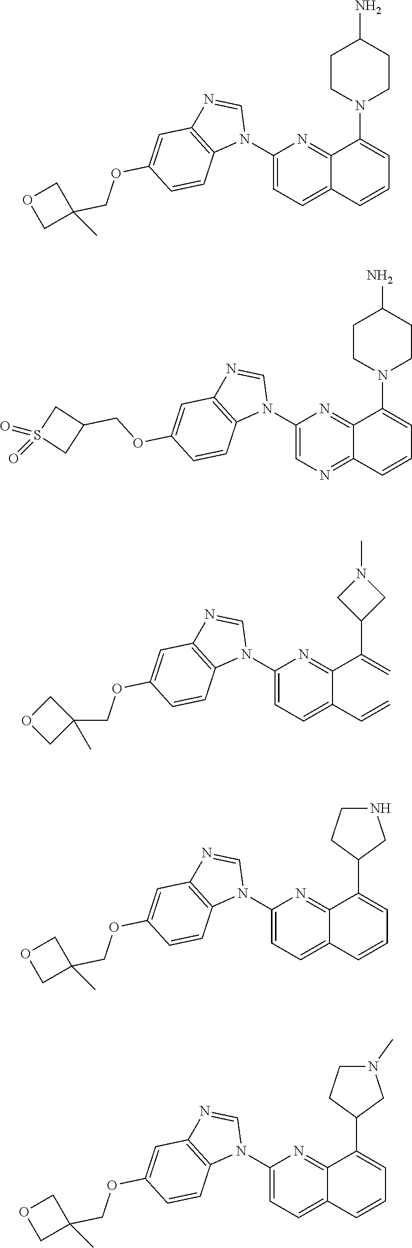 [SMILES: CC1(COc2ccc3c(c2)ncn3-c2ccc3cccc(N4CCC(N)CC4)c3n2)COC1 ; NC1CCN(c2cccc3ncc(-n4cnc5cc(OCC6CS(=O)(=O)C6)ccc54)nc23)CC1 ; C=Cc1ccc(-n2cnc3cc(OCC4(C)COC4)ccc32)nc1C(=C)C1CN(C)C1 ; CC1(COc2ccc3c(c2)ncn3-c2ccc3cccc(C4CCNC4)c3n2)COC1 ; CN1CCC(c2cccc3ccc(-n4cnc5cc(OCC6(C)COC6)ccc54)nc23)C1]

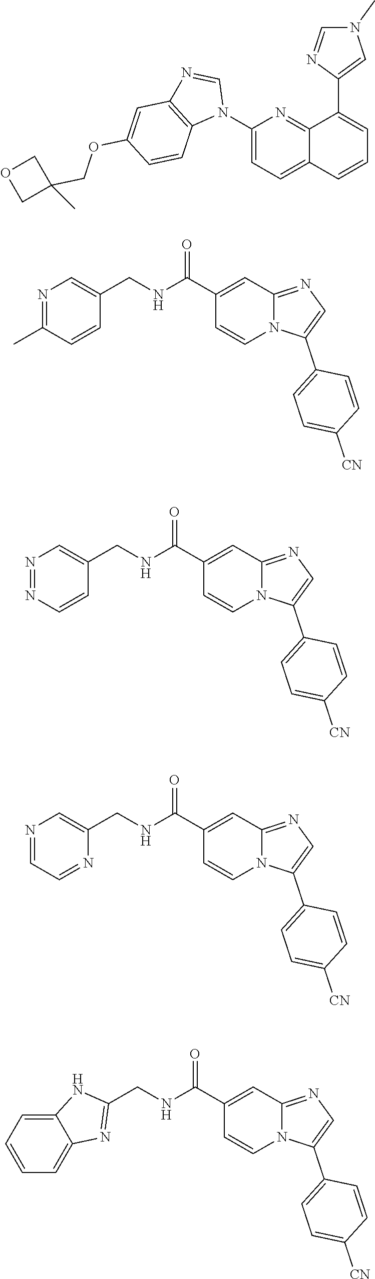

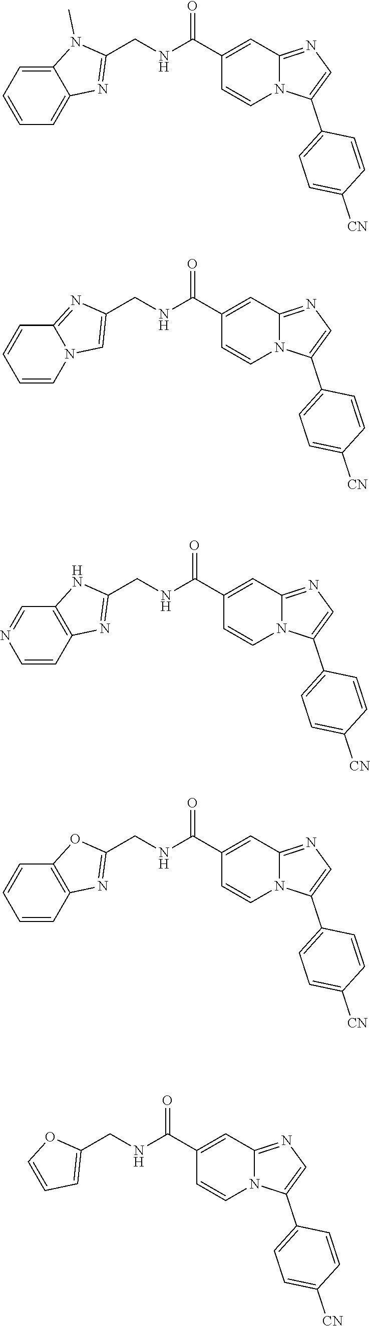 [SMILES: Cn1c(CNC(=O)c2ccn3c(-c4ccc(C#N)cc4)cnc3c2)nc2ccccc21 ; N#Cc1ccc(-c2cnc3cc(C(=O)NCc4cn5ccccc5n4)ccn23)cc1 ; N#Cc1ccc(-c2cnc3cc(C(=O)NCc4nc5ccncc5[nH]4)ccn23)cc1 ; N#Cc1ccc(-c2cnc3cc(C(=O)NCc4nc5ccccc5o4)ccn23)cc1 ; N#Cc1ccc(-c2cnc3cc(C(=O)NCc4ccco4)ccn23)cc1]

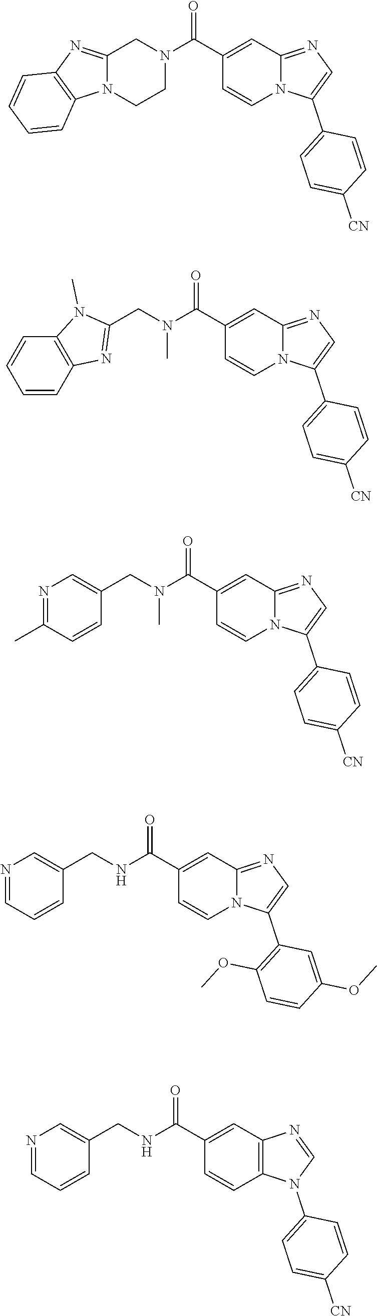 [SMILES: N#Cc1ccc(-c2cnc3cc(C(=O)N4CCn5c(nc6ccccc65)C4)ccn23)cc1 ; CN(Cc1nc2ccccc2n1C)C(=O)c1ccn2c(-c3ccc(C#N)cc3)cnc2c1 ; Cc1ccc(CN(C)C(=O)c2ccn3c(-c4ccc(C#N)cc4)cnc3c2)cn1 ; COc1ccc(OC)c(-c2cnc3cc(C(=O)NCc4cccnc4)ccn23)c1 ; N#Cc1ccc(-n2cnc3cc(C(=O)NCc4cccnc4)ccc32)cc1]

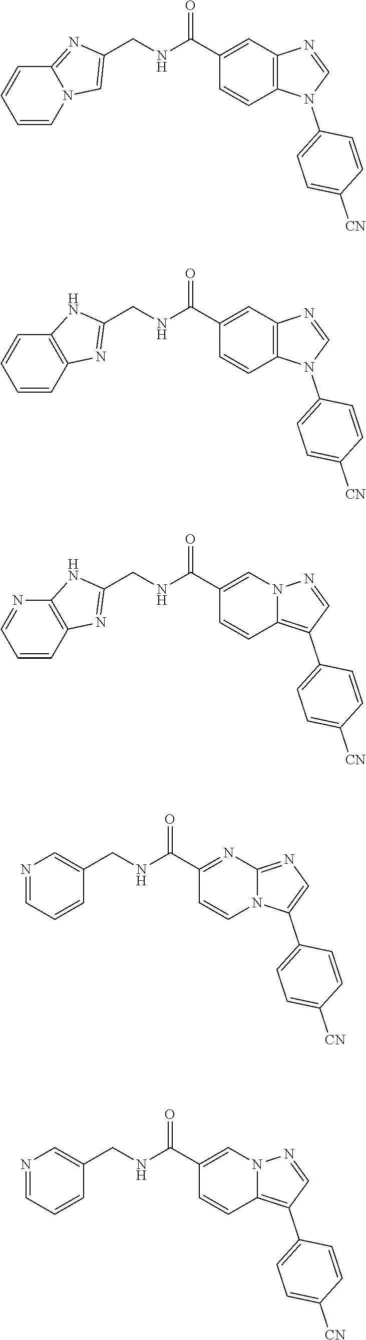 [SMILES: N#Cc1ccc(-n2cnc3cc(C(=O)NCc4cn5ccccc5n4)ccc32)cc1 ; N#Cc1ccc(-n2cnc3cc(C(=O)NCc4nc5ccccc5[nH]4)ccc32)cc1 ; N#Cc1ccc(-c2cnn3cc(C(=O)NCc4nc5cccnc5[nH]4)ccc23)cc1 ; N#Cc1ccc(-c2cnc3nc(C(=O)NCc4cccnc4)ccn23)cc1 ; N#Cc1ccc(-c2cnn3cc(C(=O)NCc4cccnc4)ccc23)cc1]

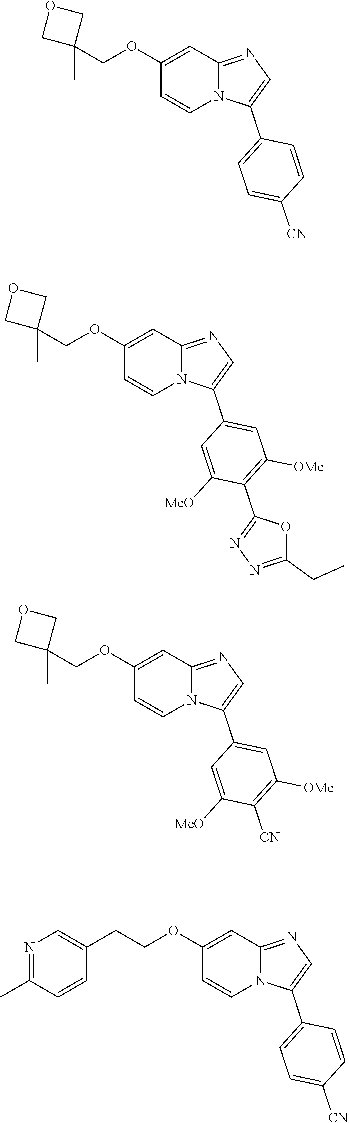

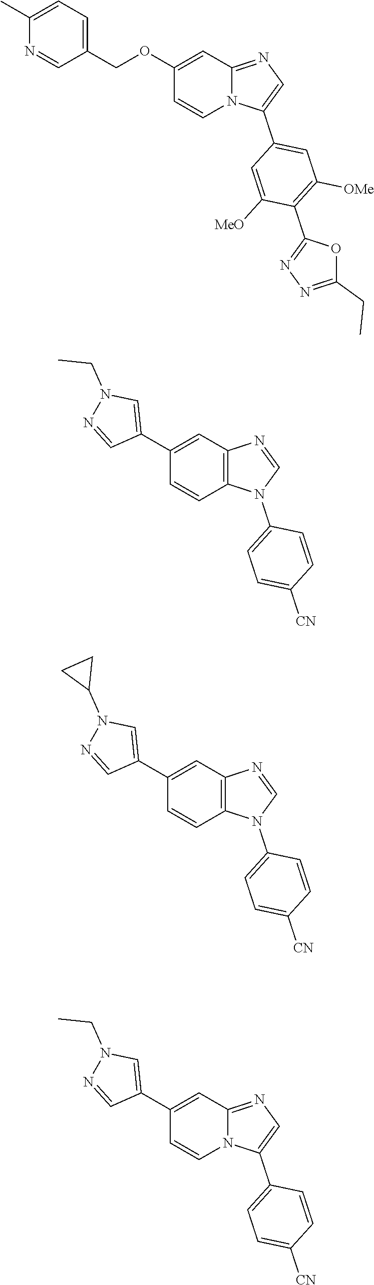 [SMILES: CCc1nnc(-c2c(OC)cc(-c3cnc4cc(OCc5ccc(C)nc5)ccn34)cc2OC)o1 ; CCn1cc(-c2ccc3c(c2)ncn3-c2ccc(C#N)cc2)cn1 ; N#Cc1ccc(-n2cnc3cc(-c4cnn(C5CC5)c4)ccc32)cc1 ; CCn1cc(-c2ccn3c(-c4ccc(C#N)cc4)cnc3c2)cn1]

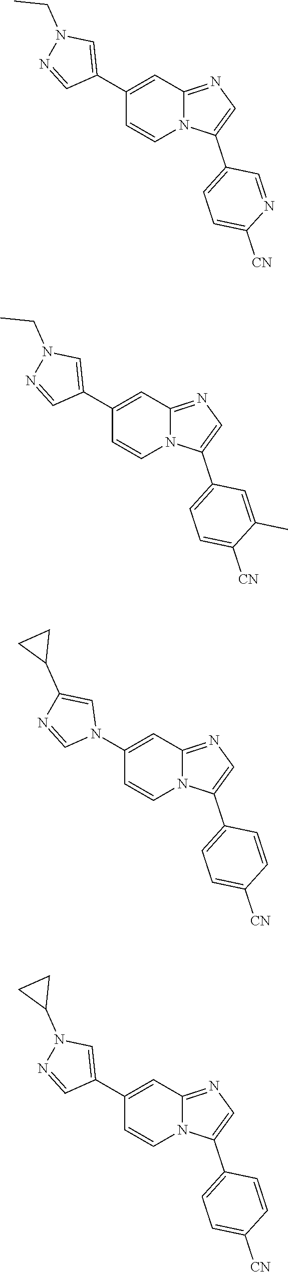 [SMILES: CCn1cc(-c2ccn3c(-c4ccc(C#N)nc4)cnc3c2)cn1 ; CCn1cc(-c2ccn3c(-c4ccc(C#N)c(C)c4)cnc3c2)cn1 ; N#Cc1ccc(-c2cnc3cc(-n4cnc(C5CC5)c4)ccn23)cc1 ; N#Cc1ccc(-c2cnc3cc(-c4cnn(C5CC5)c4)ccn23)cc1]

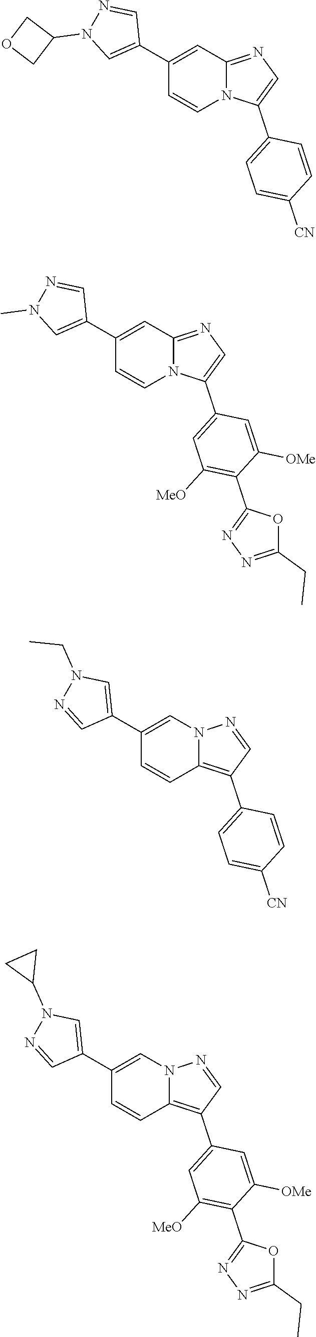 [SMILES: N#Cc1ccc(-c2cnc3cc(-c4cnn(C5COC5)c4)ccn23)cc1 ; CCc1nnc(-c2c(OC)cc(-c3cnc4cc(-c5cnn(C)c5)ccn34)cc2OC)o1 ; CCn1cc(-c2ccc3c(-c4ccc(C#N)cc4)cnn3c2)cn1 ; CCc1nnc(-c2c(OC)cc(-c3cnn4cc(-c5cnn(C6CC6)c5)ccc34)cc2OC)o1]

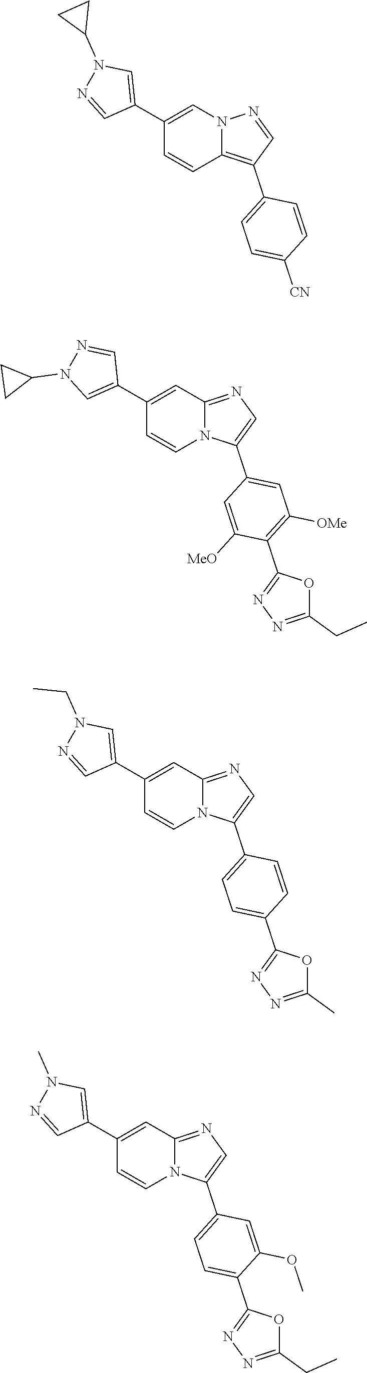

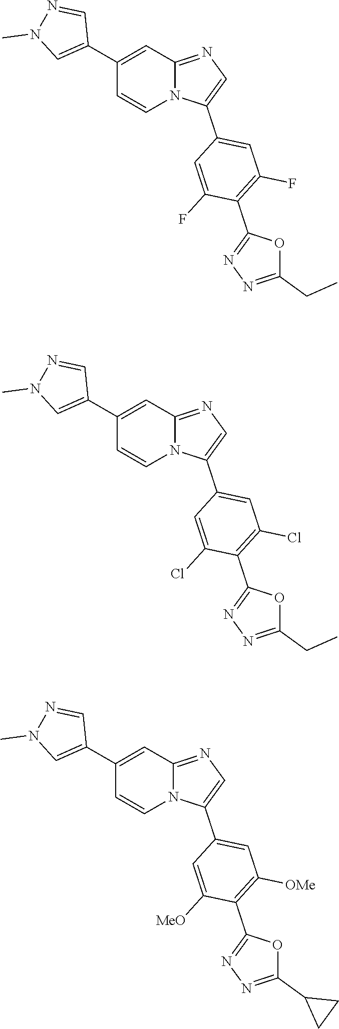

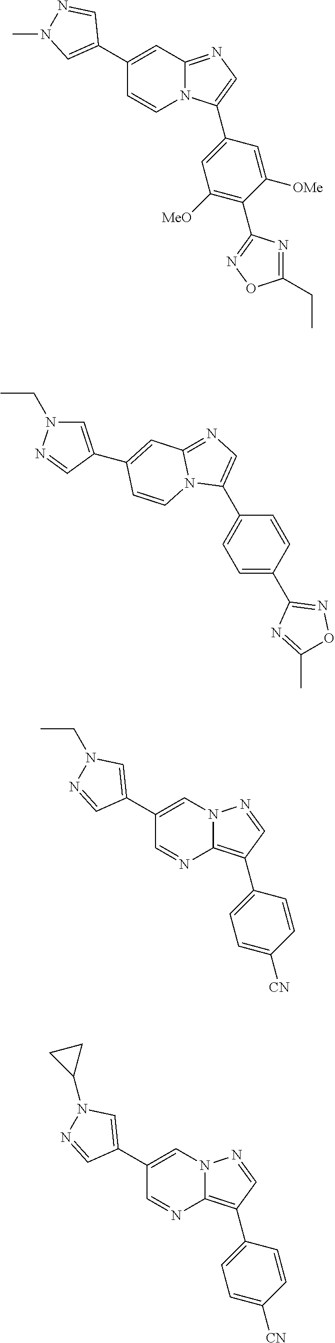

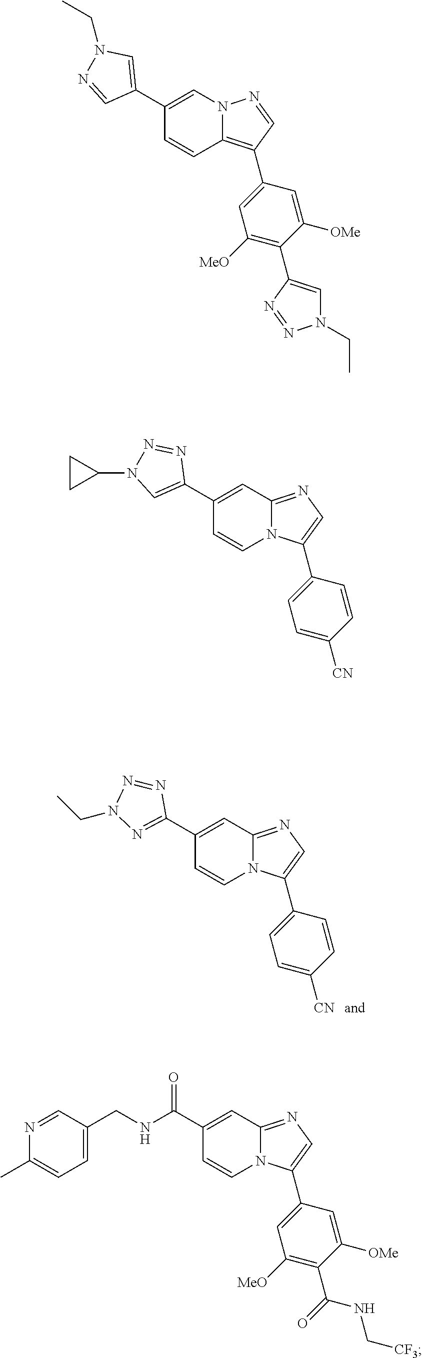

or a pharmaceutically acceptable salt thereof.

In some embodiments, the compound has formula:

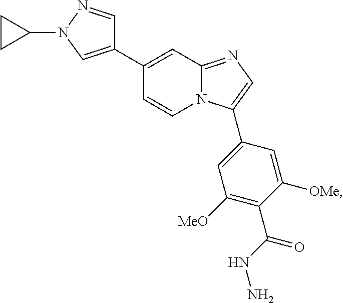 [SMILES: COc1cc(-c2cnc3cc(-c4cnn(C5CC5)c4)ccn23)cc(OC)c1C(=O)NN]

or a pharmaceutically acceptable salt thereof.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

At various places in the present specification, divalent linking substituents are described. It is specifically intended that each divalent linking substituent include both the forward and backward forms of the linking substituent. For example, —NR(CR′R″)ₙ— includes both —NR(CR′R″)ₙ— and —(CR′R″)ₙNR—. Where the structure clearly requires a linking group, the Markush variables listed for that group are understood to be linking groups.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent. e.g., oxo, can replace two hydrogen atoms. It is to be understood that substitution at a given atom is limited by valency.

As used herein, the phrase “each ‘variable’ is independently selected from” means substantially the same as wherein “at each occurrence ‘variable’ is selected from”

Throughout the definitions, the term “Cₙ₋ₘ” indicates a range which includes the endpoints, wherein n and m are integers and indicate the number of carbons. Examples include C₁₋₃, C₁₋₄, C₁₋₆, and the like.

As used herein, the term “Cn-m alkyl”, employed alone or in combination with other terms, refers to a saturated hydrocarbon group that may be straight-chain or branched, having n to m carbons. Examples of alkyl moieties include, but are not limited to, chemical groups such as methyl (Me), ethyl (Et), n-propyl (n-Pr), isopropyl (iPr), n-butyl, tert-butyl, isobutyl, sec-butyl: higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, “Cn-m alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein. “Cn-m alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “Cn-m alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, the term “amino” refers to a group of formula —NH₂.

As used herein, the term “aryl” refers to an aromatic hydrocarbon group, which may be monocyclic or polycyclic (e.g., having 2 or more fused rings). The term “Cₙ₋ₘ aryl” refers to an aryl group having from n to m ring carbon atoms. Aryl groups include, but are not limited to, phenyl, naphthyl, and the like. In some embodiments, aryl groups have from 6 to 14 carbon atoms (i.e., C₆₋₁₄ aryl). In some embodiments, aryl groups have from 6 to 10 carbon atoms (i.e., C₆₋₁₀ aryl). In some embodiments, the aryl is phenyl.

As used herein, “halo” refers to F, Cl, Br, or I. In some embodiments, a halo is F or Cl In some embodiments, a halo is F.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms. In some embodiments, the haloalkyl group is trifluoroethyl.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)amino” refers to a group of formula —N(alkyl)₂, wherein each alkyl group independently has n to m carbon atoms.

In some embodiments, each alkyl group independently has 1 to 6, 1 to 4, or 1 to 3 carbon atoms.

As used herein, “cycloalkyl” refers to non-aromatic cyclic hydrocarbons including cyclized alkyl and alkenyl groups. Cycloalkyl groups can include mono- or poly cyclic (e.g., having 2 or more fused rings) groups, spirocycles, and bridged rings (e.g., a bridged bicycloalkyl group). Ring-forming carbon atoms of a cycloalkyl group can be optionally substituted by oxo (i.e., ═O). Also included in the definition of cycloalkyl are moieties that have one or more aromatic rings fused (i.e., having a bond in common with) to the cycloalkyl ring, for example, benzo or thienyl derivatives of cyclobutane, cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, 10, 11, 12, 13, or 14 ring-forming carbons (i.e., C₃₋₁₄). In some embodiments, the cycloalkyl is a C₃₋₁₄ monocyclic or bicyclic cycloalkyl. In some embodiments, the cycloalkyl is a C₃₋₁₀ monocyclic or bicyclic cycloalkyl. In some embodiments, the cycloalkyl is a C₃₋₇ monocyclic cycloalkyl. In some embodiments, the cycloalkyl is a C₄0.7 monocyclic cycloalkyl. In some embodiments, the cycloalkyl is a C₄₋₁₄ spirocycle or bridged cycloalkyl (e.g., a bridged bicycloalkyl group). In some embodiments, the cycloalkyl is a C₄₋₁₀ spirocycle or bridged cycloalkyl (e.g., a bridged bicycloalkyl group). Example cycloalkyl groups include, but are not limited to, cyclopropyl, cyclobutyl, cyclopentyl, cyclohexyl, cycloheptyl, cyclopentenyl, cyclohexenyl, cyclohexadienyl, cycloheptatrienyl, norbomyl, adamantanyl, bicyclo[1.1.1]pentyl, bicyclo[2.1.1]hexyl, bicyclo[2.2.1]heptanyl, bicyclo[3.1.1]heptanyl, bicyclo[2.2.2]octanyl, spiro[3.3]heptanyl, and the like.

As used herein, “heteroaryl” refers to a monocyclic or polycyclic (e.g., having 2 or more fused rings) aromatic heterocycle having at least one heteroatom ring member selected from N. O, and S. In some embodiments, the heteroaryl ring has 1, 2, 3, or 4 heteroatom ring members independently selected from N, O, and S. In some embodiments, the heteroaryl ring has 1, 2, 3, or 4 heteroatom ring members independently selected from N and O. In some embodiments, the heteroaryl is a 5-14 membered monocyclic or bicyclic heteroaryl having 1, 2, 3, or 4 heteroatom ring members independently selected from N, O, and S. In some embodiments, the heteroaryl is a 5-10 membered monocyclic or bicyclic heteroaryl having 1, 2, 3, or 4 heteroatom ring members independently selected from N, O, and S. In some embodiments, the heteroaryl is a 5-14 membered monocyclic or bicyclic heteroaryl having 1, 2, 3, or 4 heteroatom ring members independently selected from N and O.

In some embodiments, the heteroaryl is a 5-10 membered monocyclic or bicyclic heteroaryl having 1, 2, 3, or 4 heteroatom ring members independently selected from N and O. In some embodiments, the heteroaryl is a 5-6 membered monocyclic heteroaryl having 1 or 2 heteroatom ring members independently selected from N, O, and S. In some embodiments, the heteroaryl is a 5-6 membered monocyclic heteroaryl having 1 or 2 heteroatom ring members independently selected from N and O. In some embodiments, the heteroaryl group has 1 to 4 ring-forming heteroatoms, 1 to 3 ring-forming heteroatoms, 1 to 2 ring-forming heteroatoms or 1 ring-forming heteroatom. When the heteroaryl group contains more than one heteroatom ring member, the heteroatoms may be the same or different. Example heteroaryl groups include, but are not limited to, thienyl (or thiophenyl), furyl (or furanyl), pyrrolyl, imidazolyl, thiazolyl, oxazolyl, pyrazolyl, isothiazolyl, isoxazolyl, 1,2,3-triazolyl, tetrazolyl, 1,2,3-thiadiazolyl. 1,2,3-oxadiazolyl, 1,2,4-triazolyl, 1,2,4-thiadiazolyl, 1.2.4-oxadiazolyl, 1,3,4-triazolyl, 1,3,4-thiadiazolyl, 1,3,4-oxadiazolyl and 1.2-dihydro-1,2-azaborine, pyridinyl, pyrimidinyl, pyrazinyl, pyridazinyl, azolyl, triazolyl, thiadiazolyl, quinolinyl, isoquinolinyl, indolyl, benzothiophenyl, benzofuranyl, benzisoxazolyl, imidazo[1,2-b]thiazolyl, purinyl, triazinyl, thieno[3,2-b]pyridinyl, imidazo[1,2-a]pyridinyl, 1,5-naphthyridinyl, 1H-pyrazolo[4,3-b]pyridinyl, triazolo[4.3-a]pyridinyl. 1H-pyrrolo[3,2-b]pyridinyl, 1H-pyrrolo[2,3-b]pyridinyl, pyrazolo[1,5-a]pyridinyl, indazolyl, and the like.

As used herein, “heterocycloalkyl” refers to monocyclic or polycyclic heterocycles having at least one non-aromatic ring (i.e., a saturated or partially unsaturated ring), wherein one or more of the ring-forming carbon atoms of the heterocycloalkyl is replaced by a heteroatom selected from N, O, and S, and wherein the ring-forming carbon atoms and heteroatoms of a heterocycloalkyl group can be optionally substituted by one or more oxo groups (i.e., substituted by one or more ═O groups, such as S(O), C(O), S(O)₂, and the like), etc.). When a ring-forming carbon atom or heteroatom of a heterocycloalkyl group is optionally substituted by one or more oxo groups, the 0 of said group is in addition to the number of ring-forming atoms specified herein. Heterocycloalkyl groups include monocyclic and polycyclic (e.g., having 2 or more fused rings) systems. Heterocycloalkyl groups provided include monocyclic and polycyclic 3 to 14 membered. 3 to 10 membered, 4 to 14 membered, 4 to 10 membered, 5 to 14 membered, 5 to 10 membered, 4 to 7 membered, 5 to 7 membered, or 5 to 6 membered heterocycloalkyl groups.

Heterocycloalkyl groups can also include spirocycles and bridged rings (e.g., a 5 to 14 membered bridged heterocycloalkyl ring having one or more of the ring-forming carbon atoms replaced by a heteroatom independently selected from N, O, and S). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 double bonds. In some embodiments, the heterocycloalkyl group contains 1, 2, or 3 double bonds.

Also included in the definition of heterocycloalkyl are moieties that have one or more aromatic rings fused (i.e., having a bond in common with) to the non-aromatic heterocyclic ring, for example, benzo or thienyl derivatives of piperidine, morpholine, azetidine, and the like. A heterocycloalkyl group containing a fused aromatic ring can be attached through any ring-forming atom including a ring-forming atom of the fused aromatic ring.

As used herein, “Cₒ₋ₚ cycloalkyl-Cₙ₋ₘ alkyl-” refers to a group of formula cycloalkyl-alkylene-, wherein the cycloalkyl has o to p carbon atoms and the alkylene linking group has n to m carbon atoms.

As used herein “Cₒ₋ₚ aryl-Cₙ₋ₘ alkyl-” refers to a group of formula aryl-alkylene-, wherein the aryl has o to p carbon atoms and the alkylene linking group has n to m carbon atoms.

As used herein, “heteroaryl-Cₙ₋ₘ alkyl-” refers to a group of formula heteroaryl-alkylene-, wherein alkylene linking group has n to m carbon atoms.

As used herein “heterocycloalkyl-Cₙ₋ₘ alkyl-” refers to a group of formula heterocycloalkyl-alkylene-, wherein alkylene linking group has n to m carbon atoms.

As used herein, an “alkylene group” is a bivalent straight chain or branched alkyl linking group. For example, “Cₒ₋ₚ cycloalkyl-Cₙ-m alkyl-”. “Cₒ₋ₚ aryl-Cₙ₋ₘ alkyl-““phenyl-Cₙ₋ₘ alkyl-”, “heteroaryl-Cₙ₋ₘ alkyl-”, and “heterocycloalkyl-Cₙ₋ₘ alkyl-” contain alkyl linking groups. Examples of “alkyl linking groups” or “alkylene groups” include methylene, ethan-1,1-diyl, ethan-1,2-diyl, propan-1,3-dilyl, propan-1,2-diyl, propan-1.1-diyl and the like.

As used herein, the term “oxo” refers to an oxygen atom (i.e., ═O) as a divalent substituent, forming a carbonyl group when attached to a carbon (i.e., C═O or C(O)), or attached to a nitrogen or sulfur heteroatom forming a nitroso, sulfinyl, or sulfonyl group.

At certain places, the definitions or embodiments refer to specific rings (e.g., an azetidine ring, a pyridine ring, etc.). Unless otherwise indicated, these rings can be attached to any ring member provided that the valency of the atom is not exceeded.

For example, an azetidine ring may be attached at any position of the ring, whereas a pyridin-3-yl ring is attached at the 3-position.

As used herein, the term “independently selected from” means that each occurrence of a variable or substituent (e.g., “each R^{1A}”), are independently selected at each occurrence from the applicable list.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present disclosure are described and may be isolated as a mixture of isomers or as separated isomeric forms. In some embodiments, the compound has the (R)-configuration. In some embodiments, the compound has the (S)-configuration. The Formulas (e.g., Formula I, Formula Ia, etc.) provided herein include stereoisomers of the compounds.

Resolution of racemic mixtures of compounds can be carried out by any of numerous methods known in the art. An example method includes fractional recrystallizaion using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as p-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example 1H- and 3H-imidazole, 1H-, 2H- and 4H-1,2,4-triazole, 1H- and 2H-isoindole, 2-hydroxypyridine and 2-pyridone, and 1H- and 2H-pyrazole. Tautomeric forms can be in equilibrium or sterically locked into one form by appropriate substitution.

All compounds, and pharmaceutically acceptable salts thereof, can be found 5 together with other substances such as water and solvents (e.g. hydrates and solvates) or can be isolated.

In some embodiments, preparation of compounds can involve the addition of acids or bases to affect, for example, catalysis of a desired reaction or formation of salt forms such as acid addition salts.

Example acids can be inorganic or organic acids and include, but are not limited to, strong and weak acids. Some example acids include hydrochloric acid, hydrobromic acid, sulfuric acid, phosphoric acid, p-toluenesulfonic acid, 4-nitrobenzoic acid, methanesulfonic acid, benzenesulfonic acid, trifluoroacetic acid, and nitric acid. Some weak acids include, but are not limited to acetic acid, propionic acid, butanoic acid, benzoic acid, tartaric acid, pentanoic acid, hexanoic acid, heptanoic acid, octanoic acid, nonanoic acid, and decanoic acid.

Example bases include lithium hydroxide, sodium hydroxide, potassium hydroxide, lithium carbonate, sodium carbonate, potassium carbonate, and sodium bicarbonate. Some example strong bases include, but are not limited to, hydroxide, alkoxides, metal amides, metal hydrides, metal dialkylamides and arylamines, wherein; alkoxides include lithium, sodium and potassium salts of methyl, ethyl and t-butyl oxides; metal amides include sodium amide, potassium amide and lithium amide; metal hydrides include sodium hydride, potassium hydride and lithium hydride; and metal dialkylamides include lithium, sodium, and potassium salts of methyl, ethyl, n-propyl, iso-propyl, n-butyl, tert-butyl, trimethylsilyl and cyclohexyl substituted amides.

As used herein, the terms “room temperature” or “RT” are understood in the art, and refer generally to a temperature (e.g., a reaction temperature) that is about the temperature of the room in which the reaction is carried out, for example, a temperature from about 20° C. to about 30° C.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### Synthesis

As will be appreciated by those skilled in the art, the compounds provided herein, including salts and stereoisomers thereof, can be prepared using known organic synthesis techniques and can be synthesized according to any of numerous possible synthetic routes.

The compounds provided herein, or intermediates useful in the preparation of the compounds provided herein, can be prepared, for example, according to the procedures described in WO 2022/031928, which is incorporated herein by reference in its entirety.

A skilled artisan will appreciate that the processes described above are not the exclusive means by which compounds provided herein may be synthesized and that a broad repertoire of synthetic organic reactions is available to be potentially employed in synthesizing compounds provided herein. A skilled artisan knows how to select and implement appropriate synthetic routes. Suitable synthetic methods of starting materials, intermediates and products may be identified by reference to the literature, including reference sources such as: Advances in Heterocyclic Chemistry, Vols. 1-107 (Elsevier, 1963-2012); Journal of Heterocyclic Chemistry Vols. 1-49 (Journal of Heterocyclic Chemistry, 1964-2012); Carreira, et al. (Ed.) Science of Synthesis, Vols. 1-48 (2001-2010) and Knowledge Updates KU2010/1-4; 2011/1-4; 2012/1-2 (Thieme, 2001-2012); Katritzky, et al. (Ed.) Comprehensive Organic Functional Group Transformations, (Pergamon Press, 1996); Katritzky et al. (Ed.); Comprehensive Organic Functional Group Transformations II (Elsevier, 2^{nd} Edition, 2004); Katritzky et al. (Ed.), Comprehensive Heterocyclic Chemistry (Pergamon Press, 1984); Katritzky et al., Comprehensive Heterocyclic Chemistry II, (Pergamon Press, 1996); Smith et al., March's Advanced Organic Chemistry: Reactions, Mechanisms, and Structure, 6^{th} Ed. (Wiley, 2007); Trost et al. (Ed.), Comprehensive Organic Synthesis (Pergamon Press, 1991).

Preparation of compounds described herein can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3^{rd} Ed., Wiley & Sons, Inc., New York (1999).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC).

Compounds can be purified by those skilled in the art by a variety of methods, including high performance liquid chromatography (HPLC) and normal phase silica chromatography.

### 2-[2,6-Dimethoxy-4-[7-(1-methylpyrazol-4-yl)imidazo[1,2-a]pyridin-3-yl]phenyl]-5-ethyl-1,3,4-oxadiazole

In some embodiments, a selective SIK2/SIK3 inhibitor for use in the methods of the present disclosure is 2-[2,6-Dimethoxy-4-[7-(1-methylpyrazol-4-yl)imidazo[1,2-a]pyridin-3-yl]phenyl]-5-ethyl-1,3,4-oxadiazole, or a pharmaceutically acceptable salt thereof. For example, a selective SIK2/SIK3 inhibitor for use in the present methods can be a compound with the structure:

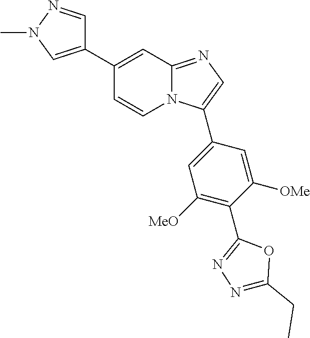 [SMILES: CCc1nnc(-c2c(OC)cc(-c3cnc4cc(-c5cnn(C)c5)ccn34)cc2OC)o1]

or a pharmaceutically acceptable salt thereof. In some embodiments, 2-[2,6-Dimethoxy-4-[7-(1-methylpyrazol-4-yl)imidazo[1,2-a]pyridin-3-yl]phenyl]-5-ethyl-1,3,4-oxadiazole (or a pharmaceutically acceptable salt thereof) is referred to herein as SK-124. Detailed description regarding 2-[2,6-Dimethoxy-4-[7-(1-methylpyrazol-4-yl)imidazo[1,2-a]pyridin-3-yl]phenyl]-5-ethyl-1,3,4-oxadiazole (or a pharmaceutically acceptable salt thereof) and synthesis of the same is provided, e.g., in WO 2022/031928, which is incorporated herein by reference in its entirety. In some embodiments, 2-[2,6-Dimethoxy-4-[7-(1-methylpyrazol-4-yl)imidazo[1,2-a]pyridin-3-yl]phenyl]-5-ethyl-1,3,4-oxadiazole (or a pharmaceutically acceptable salt thereof) is synthesized, as described in WO 2022/031928, e.g., in Example 51.

### Antibodies

The methods described herein can include the use of antibodies to one or more SIK proteins. e.g., human SIK proteins. Thus, in some embodiments, the present disclosure provides antibodies that selectively inhibit one or more SIKs. For example, described herein are antibodies that selectively bind to and inhibit SIK 2 and SIK3, over SIK1 (e.g., antibodies that are selective SIK2/SIK3 inhibitors). The term “antibody” as used herein refers to an immunoglobulin molecule or an antigen-binding portion thereof. Examples of antigen-binding portions of immunoglobulin molecules include F(ab) and F(ab′)₂ fragments, which retain the ability to bind antigen. The antibody can be polyclonal, monoclonal, recombinant, chimeric, de-immunized or humanized, fully human, non-human. (e.g., murine), or single chain antibody. In some embodiments the antibody has effector function and can fix complement. In some embodiments, the antibody has reduced or no ability to bind an Fc receptor. For example, the antibody can be an isotype or subtype, fragment or other mutant, which does not support binding to an Fc receptor, e.g., it has a mutagenized or deleted Fc receptor binding region. Methods for making antibodies and fragments thereof are known in the art, see, e.g., Harlow et. al., editors, Antibodies: A Laboratory Manual (1988); Goding, Monoclonal Antibodies: Principles and Practice, (N.Y. Academic Press 1983); Howard and Kaser, Making and Using Antibodies: A Practical Handbook (CRC Press; 1st edition, Dec 13, 2006); Kontermann and Dubel, Antibody Engineering Volume 1 (Springer Protocols) (Springer; 2nd ed., May 21, 2010); Lo, Antibody Engineering: Methods and Protocols (Methods in Molecular Biology) (Humana Press; Nov. 10, 2010); and DUbel, Handbook of Therapeutic Antibodies: Technologies, Emerging Developments and Approved Therapeutics, (Wiley-VCH: 1 edition Sep. 7, 2010). Antibodies useful in the present methods include those that bind specifically to (i.e., do not bind to targets other than) SIK2 and SIK3. Such antibodies can ameliorate epithelial cell injury and treat chronic kidney disease. Anti-SIK antibodies are also commercially available, e.g., from AAT Bioquest, Inc.; Abcam; Biorbyt; Cell Signaling Technology; G Biosciences; GeneTex; and MyBioSource.com.

In some embodiments, the antibody can be coupled to a detectable or imaging agent. Such agents are well known in the art and include paramagnetic agents, bioluminescent or fluorescent labels (e.g., GFP, FITC, rhodamine, or Texas Red), radioactive isotopes, and colorimetric/enzvmatic agents (e.g., HRP, B-galactosidase). In a preferred embodiment, the antibody is coupled to a paramagnetic agent, e.g., a paramagnetic nanoparticle, e.g., cross-linked iron oxide (CLIO) nanoparticles; see, e.g., US 20110046004; Josephson et al., Bioconjug. Chem., 10(2):186-91 (1999).

### Inhibitory Nucleic Acids

The methods described herein can include the use of inhibitory nucleic acids directed to one or more human SIKs. Thus, in some embodiments, the present disclosure provides inhibitory nucleic acids that selectively inhibit one or more SIKs. For example, described herein are inhibitory nucleic acids that selectively inhibit SIK 2 and SIK3, over SIK (e.g., inhibitory nucleic acids that are selective SIK2/SIK3 inhibitors). Inhibitory nucleic acids useful in the present methods and compositions include antisense oligonucleotides, siRNA compounds, single- or double-stranded RNA interference (RNAi) compounds such as siRNA compounds, modified bases/locked nucleic acids (LNAs), peptide nucleic acids (PNAs), and other oligomeric compounds or oligonucleotide mimetics which hybridize to at least a portion of the target SIK2/SIK3 nucleic acid and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a short, hairpin RNA (shRNA); or combinations thereof. See, e.g., WO 2010040112.

In some embodiments, the inhibitory nucleic acids are 10 to 50, 10 to 20, 10 to 25, 13 to 50, or 13 to 30 nucleotides in length. One having ordinary skill in the art will appreciate that this embodies inhibitory nucleic acids having complementary portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therewithin. In some embodiments, the inhibitory nucleic acids are 15 nucleotides in length. In some embodiments, the inhibitory nucleic acids are 12 or 13 to 20, 25, or 30 nucleotides in length. One having ordinary skill in the art will appreciate that this embodies inhibitory nucleic acids having complementary portions of 12, 13, 14, 15, 16, 17, 18, 19, 20, 21, 22, 23, 24, 25, 26, 27, 28, 29 or 30 nucleotides in length, or any range therewithin (complementary portions refers to those portions of the inhibitory nucleic acids that are complementary to the target sequence).

The inhibitory nucleic acids useful in the present methods are sufficiently complementary to the target RNA (SIK2/SIK3 mRNA), i.e., hybridize sufficiently well and with sufficient specificity, to give the desired effect. “Complementary” refers to the capacity for pairing, through hydrogen bonding, between two sequences comprising naturally or non-naturally occurring bases or analogs thereof. For example, if a base at one position of an inhibitory nucleic acid is capable of hydrogen bonding with a base at the corresponding position of a RNA, then the bases are considered to be complementary to each other at that position. 100% complementarity is not required.

Routine methods can be used to design an inhibitory nucleic acid that binds to the SIK2/SIK3 sequence with sufficient specificity. In some embodiments, the methods include using bioinformatics methods known in the art to identify regions of secondary structure, e.g., one, two, or more stem-loop structures, or pseudoknots, and selecting those regions to target with an inhibitory nucleic acid. For example, -gene walk” methods can be used to optimize the inhibitory activity of the nucleic acid; for example, a series of oligonucleotides of 10-30 nucleotides spanning the length of a target RNA can be prepared, followed by testing for activity. Optionally, gaps, e.g., of 5-10 nucleotides or more, can be left between the target sequences to reduce the number of oligonucleotides synthesized and tested. GC content is preferably between about 30-60%. Contiguous runs of three or more Gs or Cs should be avoided where possible (for example, it may not be possible with very short (e.g., about 9-10 nt) oligonucleotides).

In some embodiments, the inhibitory nucleic acid molecules can be designed to target a specific region of the RNA sequence. For example, a specific functional region can be targeted, e.g., a region comprising a known RNA localization motif (i.e., a region complementary to the target nucleic acid on which the RNA acts).

Alternatively, or in addition, highly conserved regions can be targeted, e.g., regions identified by aligning sequences from disparate species such as primate (e.g., human) and rodent (e.g., mouse) and looking for regions with high degrees of identity. Percent identity can be determined routinely using basic local alignment search tools (BLAST programs) (Altschul et al., J. Mol. Biol., 1990, 215, 403-410; Zhang and Madden, Genome Res., 1997, 7, 649-656), e.g., using the default parameters.

Once one or more target regions, segments or sites have been identified, e.g., within a SIK2/SIK3 sequence known in the art or provided herein, inhibitory nucleic acid compounds are chosen that are sufficiently complementary to the target, i.e., that hybridize sufficiently well and with sufficient specificity (i.e., do not substantially bind to other non-target RNAs), to give the desired effect.

In the context of this invention, hybridization means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. Complementary, as used herein, refers to the capacity for precise pairing between two nucleotides. For example, if a nucleotide at a certain position of an oligonucleotide is capable of hydrogen bonding with a nucleotide at the same position of a RNA molecule, then the inhibitory nucleic acid and the RNA are considered to be complementary to each other at that position. The inhibitory nucleic acids and the RNA are complementary to each other when a sufficient number of corresponding positions in each molecule are occupied by nucleotides which can hydrogen bond with each other. Thus, “specifically hybridisable” and “complementary” are terms which are used to indicate a sufficient degree of complementarity or precise pairing such that stable and specific binding occurs between the inhibitory nucleic acid and the RNA target. For example, if a base at one position of an inhibitory nucleic acid is capable of hydrogen bonding with a base at the corresponding position of a RNA, then the bases are considered to be complementary to each other at that position. 100% complementarity is not required.

It is understood in the art that a complementary nucleic acid sequence need not be 100% complementary to that of its target nucleic acid to be specifically hybridisable. A complementary nucleic acid sequence for purposes of the present methods is specifically hybridisable when binding of the sequence to the target RNA molecule interferes with the normal function of the target RNA to cause a loss of activity, and there is a sufficient degree of complementarity to avoid non-specific binding of the sequence to non-target RNA sequences under conditions in which specific binding is desired, e.g., under physiological conditions in the case of in vivo assays or therapeutic treatment, and in the case of in vitro assays, under conditions in which the assays are performed under suitable conditions of stringency. For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent. e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 300 C in 750 mM NaCl, 75 mM trisodium citrate, and 1% SDS. In a more preferred embodiment, hybridization will occur at 370 C in 500 mM NaCl. 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 250 C, more preferably of at least about 42° C., and even more preferably of at least about 680 C. In a preferred embodiment, wash steps will occur at 25° C. in 30 mM NaCl, 3 mM trisodium citrate, and 0.1% SDS. In a more preferred embodiment, wash steps will occur at 420 C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS.

Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

In general, the inhibitory nucleic acids useful in the methods described herein have at least 80% sequence complementarity to a target region within the target nucleic acid, e.g., 90%, 95%, or 100% sequence complementarity to the target region within an RNA. For example, an antisense compound in which 18 of 20 nucleobases of the antisense oligonucleotide are complementary, and would therefore specifically hybridize, to a target region would represent 90 percent complementarity. Percent complementarity of an inhibitory nucleic acid with a region of a target nucleic acid can be determined routinely using basic local alignment search tools (BLAST programs) (Altschul et al., J. Mol. Biol., 1990, 215, 403-410; Zhang and Madden, Genome Res., 1997, 7, 649-656). Inhibitory nucleic acids that hybridize to an RNA can be identified through routine experimentation. In general, the inhibitory nucleic acids must retain specificity for their target, i.e., must not directly bind to, or directly significantly affect expression levels of, transcripts other than the intended target.

For further disclosure regarding inhibitory nucleic acids, please see US2010/0317718 (antisense oligos); US2010/0249052 (double-stranded ribonucleic acid (dsRNA)): US2009/0181914 and US2010/0234451 (LNAs); US2007/0191294 (siRNA analogues); US2008/0249039 (modified siRNA); and WO2010/129746 and WO2010/040112 (inhibitory nucleic acids).

### Antisense Oligonucleotides

In some embodiments, the inhibitory nucleic acids used in the methods described herein are antisense oligonucleotides. For example, described herein are antisense oligonucleotides that selectively inhibit SIK 2 and SIK3, over SIK1 (e.g., antisense oligonucleotides that are selective SIK2/SIK3 inhibitors). Antisense oligonucleotides are typically designed to block expression of a DNA or RNA target by binding to the target and halting expression at the level of transcription, translation, or splicing. Antisense oligonucleotides of the present disclosure can be complementary nucleic acid sequences designed to hybridize under stringent conditions to an RNA. Thus, oligonucleotides are chosen that are sufficiently complementary to the target, i.e., that hybridize sufficiently well and with sufficient specificity, to give the desired effect.

siRNA/shRNA

In some embodiments, the inhibitory nucleic acids used in the methods described herein are interfering RNAs. Thus, in some embodiments, the nucleic acid sequence that is complementary to SIK2/SIK3 RNA is interfering RNA, including but not limited to a small interfering RNA (“siRNA”) and a small hairpin RNA (“shRNA”). For example, described herein are siRNAs that selectively inhibit SIK 2 and SIK3, over SIK1 (e.g., siRNAs that are selective SIK2/SIK3 inhibitors). Additionally, or in the alternative, described herein are shRNAs that selectively inhibit SIK 2 and SIK3, over SIK1 (e.g., shRNAs that are selective SIK2/SIK3 inhibitors).

Methods for constructing interfering RNAs are well known in the art. For example, the interfering RNA can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure); the antisense strand comprises nucleotide sequence that is complementary to a nucleotide sequence in a target nucleic acid molecule or a portion thereof (i.e., an undesired gene) and the sense strand comprises nucleotide sequence corresponding to the target nucleic acid sequence or a portion thereof. Alternatively, interfering RNA is assembled from a single oligonucleotide, where the self-complementary sense and antisense regions are linked by means of nucleic acid based or non-nucleic acid-based linker(s). The interfering RNA can be a polynucleotide with a duplex, asymmetric duplex, hairpin or asymmetric hairpin secondary structure, having self-complementary sense and antisense regions, wherein the antisense region comprises a nucleotide sequence that is complementary to nucleotide sequence in a separate target nucleic acid molecule or a portion thereof and the sense region having nucleotide sequence corresponding to the target nucleic acid sequence or a portion thereof. The interfering can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNA interference.

In some embodiments, the interfering RNA coding region encodes a self-complementary RNA molecule having a sense region, an antisense region and a loop region. Such an RNA molecule when expressed desirably forms a “hairpin” structure, and is referred to herein as an “shRNA.” The loop region is generally between about 2 and about 10 nucleotides in length. In some embodiments, the loop region is from about 6 to about 9 nucleotides in length. In some embodiments, the sense region and the antisense region are between about 15 and about 20 nucleotides in length. Following post-transcriptional processing, the small hairpin RNA is converted into a siRNA by a cleavage event mediated by the enzyme Dicer, which is a member of the RNase III family. The siRNA is then capable of inhibiting the expression of a gene with which it shares homology. For details, see Brummelkamp et al., Science 296:550-553, (2002); Lee et al, Nature Biotechnol., 20, 500-505, (2002); Miyagishi and Taira, Nature Biotechnol 20:497-500, (2002); Paddison et al. Genes & Dev. 16:948-958, (2002); Paul, Nature Biotechnol, 20, 505-508, (2002); Sui. Proc. Natl. Acad. Sd. USA, 99(6), 5515-5520, (2002); Yu et al. Proc Natl Acad Sci USA 99:6047-6052. (2002).

The target RNA cleavage reaction guided by siRNAs is highly sequence specific. In general, siRNA containing nucleotide sequences identical to a portion of the target nucleic acid are preferred for inhibition. However, 100% sequence identity between the siRNA and the target gene is not required to practice the present invention. Thus, the present disclosure has the advantage of being able to tolerate sequence variations that might be expected due to genetic mutation, strain polymorphism, or evolutionary divergence. For example, siRNA sequences with insertions, deletions, and single point mutations relative to the target sequence have also been found to be effective for inhibition. Alternatively, siRNA sequences with nucleotide analog substitutions or insertions can be effective for inhibition. In general, the siRNAs must retain specificity for their target, i.e., must not directly bind to, or directly significantly affect expression levels of, transcripts other than the intended target.

### Modified Inhibitory Nucleic Acids

In some embodiments, the inhibitory nucleic acids used in the methods described herein are modified, e.g., comprise one or more modified bonds or bases. For example, described herein are modified inhibitory nucleic acids that selectively inhibit SIK 2 and SIK3, over SIK1 (e.g., modified inhibitory nucleic acid molecules that are selective SIK2/SIK3 inhibitors).

A number of modified bases include phosphorothioate, methylphosphonate, peptide nucleic acids, or locked nucleic acid (LNA) molecules. Some inhibitory nucleic acids are fully modified, while others are chimeric and contain two or more chemically distinct regions, each made up of at least one nucleotide. These inhibitory nucleic acids typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Also, for use in the present methods, chimeric inhibitory nucleic acids can be formed as composite structures of two or more oligonucleotides, modified oligonucleotides, oligonucleosides and/or oligonucleotide mimetics as described above. Such compounds have also been referred to in the art as hybrids or gapmers. In some embodiments, the oligonucleotide is a gapmer (contain a central stretch (gap) of DNA monomers sufficiently long to induce RNase H cleavage, flanked by blocks of LNA modified nucleotides; see, e.g., Stanton et al., Nucleic Acid Ther. 2012, 22: 344-359; Nowotny et al., Cell, 121:1005-1016, 2005; Kurreck, European Journal of Biochemistry 270:1628-1644, 2003; Fluiter et al., Mol Biosyst. 5(8):838-43, 2009). In some embodiments, the oligonucleotide is a mixmer (includes alternating short stretches of LNA and DNA; Naguibneva et al., Biomed Pharmacother. 2006 Nov: 60(9):633-8; Orom et al., Gene. 2006 May 10; 372( ):137-41). Representative United States patents that teach the preparation of such hybrid structures include, but are not limited to, U.S. Pat. Nos. 5,013,830; 5,149,797: 5, 220,007; 5,256,775: 5,366,878; 5,403.711; 5,491,133: 5,565,350: 5,623,065; 5,652,355; 5,652.356; and 5,700,922, each of which is herein incorporated by reference.

In some embodiments, an inhibitory nucleic acid for use in the present methods comprises at least one nucleotide modified at the 2′ position of the sugar, most preferably a 2′-O-alkyl, 2′-O-alkyl-O-alkyl or 2′-fluoro-modified nucleotide. In some embodiments, RNA modifications include 2′-fluoro, 2-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than; 2′-deoxyoligonucleotides against a given target.

A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH2—NH—O—CH2, CH, ~N(CH3)~O—CH2 (known as a methylene(methylimino) or MMI backbone], CH2—O—N(CH3)—CH2, CH2—N(CH3)—N(CH3)—CH2 and O—N(CH3)—CH2—CH2 backbones, wherein the native phosphodiester backbone is represented as O—P—O—CH,): amide backbones (see De Mesmaeker et al. Ace. Chem. Res. 1995, 28:366-374); morpholino backbone structures (see, Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′: see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220: Lacerra et al., Proc. Natl. Acad. Sci., 2000, 97, 9591-9596; and U.S. Pat. No. 5,034,506, issued Jul. 23, 1991.

Cyclohexenyl nucleic acid oligonucleotide mimetics are described in Wang et al., J. Am. Chem. Soc., 2000, 122, 8595-8602.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts; see U.S. Pat. Nos. 5,034,506; 5,166,315; 5,185,444; 5,214,134; 5,216,141; 5,235,033; 5,264, 562; 5, 264,564; 5,405,938; 5.434,257; 5,466,677; 5,470.967; 5,489,677; 5.541,307; 5.561.225; 5,596, 086; 5,602.240; 5,610.289; 5,602,240: 5,608,046; 5,610,289; 5,618,704: 5,623, 070; 5,663,312: 5,633,360; 5,677,437; and 5,677,439, each of which is herein incorporated by reference.

One or more substituted sugar moieties can also be included, e.g., one of the following at the 2′ position: OH, SH, SCH₃, F, OCN, OCH₃ OCH₃, OCH₃O(CH₂)ₙ CH₃, O(CH₂)ₙ NH₂ or O(CH₂)ₙ CH₃ where n is from 1 to about 10; Ci to C10 lower alkyl, alkoxyalkoxy, substituted lower alkyl, alkaryl or aralkyl; C1; Br; CN; CF3; OCF3; O—, S—, or N-alkyl; O—, S—, or N-alkenyl; SOCH3; SO2 CH₃; ONO2; NO2; N3; NH2; heterocycloalkyl; heterocycloalkaryl; aminoalkylamino; polyalkylamino: substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-O—CH₂CH₂OCH₃, also known as 2′-O-(2-methoxyethyl)](Martin et al, HeIv. Chim. Acta, 1995, 78, 486). Other preferred modifications include 2′-methoxy (2′-O—CH₃), 2′-propoxy (2′-OCH₂ CH₂CH₃) and 2′-fluoro (2-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Inhibitory nucleic acids for use in the present methods can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g.. 2-aminoadenine. 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine and 2.6-diaminopurine. Kornberg, A., DNA Replication, W. H. Freeman & Co., San Francisco, 1980, pp 75-77; Gebeyehu, G., et al. Nucl. Acids Res. 1987, 15:4513). A “universal” base known in the art, e.g., inosine, can also be included. 5-Me-C substitutions have been shown to increase nucleic acid duplex stability by 0.6-1.2<0>C. (Sanghyi, Y. S., in Crooke, S. T, and Lebleu, B., eds., Antisense Research and Applications, CRC Press, Boca Raton. 1993, pp. 276-278) and are presently preferred base substitutions.

It is not necessary for all positions in a given oligonucleotide to be uniformly modified, and in fact more than one of the aforementioned modifications may be incorporated in a single oligonucleotide or even at within a single nucleoside within an oligonucleotide.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds comprise, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

Inhibitory nucleic acids for use in the present methods can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C). 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl. 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

Further, nucleobases comprise those disclosed in U.S. Pat. No. 3,687,808, those disclosed in ‘The Concise Encyclopedia of Polymer Science And Engineering’, pages 858-859, Kroschwitz, J. I., ed. John Wiley & Sons, 1990, those disclosed by Englisch et al., Angewandle Chemie, International Edition’, 1991, 30, page 613, and those disclosed by Sanghyi. Y. S.. Chapter 15, Antisense Research and Applications’, pages 289-302, Crooke, S. T, and Lebleu, B, ea., CRC Press, 1993. Certain of these nucleobases are particularly useful for increasing the binding affinity of the oligomeric compounds of the invention. These include 5-substituted pyrimidines, 6-azapyrimidines and N-2, N-6 and 0-6 substituted purines, comprising 2-aminopropyladenine, 5-propynyluracil and 5-propynylcytosine. 5-methylcytosine substitutions have been shown to increase nucleic acid duplex stability by 0.6-1.2<0>C (Sanghyi, Y. S., Crooke, S. T, and Lebleu, B., eds, ‘Antisense Research and Applications’, CRC Press, Boca Raton, 1993, pp. 276-278) and are presently preferred base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175, 273; 5, 367,066; 5.432,272; 5,457,187; 5,459.255; 5,484,908; 5,502,177; 5,525,711; 5.552,540; 5,587,469; 5.596,091; 5,614.617; 5,750.692, 5,681.941, each of which is herein incorporated by reference.

In some embodiments, the inhibitory nucleic acids for use in the present methods are chemically linked to one or more moieties or conjugates that enhance the activity, cellular distribution, or cellular uptake of the oligonucleotide. Such moieties can include, but are not limited to, lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al, Ann. N. Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Kabanov et al., FEBS Lett.. 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Mancharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-t oxycholesterol moiety (Crooke et al., J. Pharmacol. Exp. Ther., 1996, 277, 923-937). See also U.S. Pat. Nos. 4,828,979; 4,948,882; 5,218,105; 5,525,465; 5,541,313; 5,545,730: 5,552, 538; 5,578,717, 5,580,731; 5,580,731; 5,591,584; 5,109,124; 5,118,802; 5,138,045; 5,414,077; 5,486, 603; 5,512,439; 5.578,718; 5,608,046; 4,587,044; 4,605,735; 4,667,025; 4,762, 779; 4,789,737; 4,824,941; 4.835,263; 4,876.335; 4,904.582; 4,958,013: 5,082, 830; 5,112,963: 5,214,136; 5,082,830: 5,112,963: 5,214,136; 5, 245,022: 5,254,469; 5.258,506; 5,262,536; 5,272,250; 5,292,873; 5,317,098; 5,371,241, 5,391, 723; 5,416,203, 5,451,463; 5,510,475; 5,512,667; 5,514,785; 5, 565,552; 5,567,810; 5,574,142; 5,585,481; 5,587,371; 5,595.726; 5,597,696; 5,599,923: 5,599, 928 and 5,688,941, each of which is herein incorporated by reference.

These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat.

Nos. 4,828.979; 4,948,882; 5,218,105: 5,525,465; 5,541,313; 5.545,730; 5,552.538; 5,578,717, 5,580,731; 5,580,731; 5,591.584; 5,109,124: 5,118,802: 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4.958,013; 5,082,830; 5,112,963; 5,214,136: 5,082,830; 5,112,963; 5,214,136; 5,245,022; 5.254,469; 5,258.506; 5,262.536; 5,272,250: 5,292,873; 5,317,098; 5,371,241, 5,391,723: 5,416,203, 5,451,463; 5.510,475; 5,512,667; 5,514.785; 5,565,552; 5,567,810; 5,574,142; 5,585,481; 5,587,371; 5,595,726; 5,597,696; 5,599,923; 5,599,928 and 5,688,941.

### Locked Nucleic Acids (LNAs)

In some embodiments, the modified inhibitory nucleic acids used in the methods described herein comprise locked nucleic acid (LNA) molecules, e.g., including [alpha]-L-LNAs. For example, described herein are LNAs that selectively inhibit SIK 2 and SIK3, over SIK1 (e.g.. LNA molecules that are selective SIK2/SIK3 inhibitors).

LNAs comprise ribonucleic acid analogues wherein the ribose ring is “locked” by a methylene bridge between the 2′-oxygen and the 4′-carbon—i.e., oligonucleotides containing at least one LNA monomer, that is, one 2′-O,4′-C-methylene β-D-ribofuranosyl nucleotide. LNA bases form standard Watson-Crick base pairs but the locked configuration increases the rate and stability of the basepairing reaction (Jepsen et al., Oligonucleotides, 14, 130-146 (2004)). LNAs also have increased affinity to base pair with RNA as compared to DNA. These properties render LNAs especially useful as probes for fluorescence in situ hybridization (FISH) and comparative genomic hybridization, as knockdown tools for miRNAs, and as antisense oligonucleotides to target mRNAs or other RNAs. e.g., RNAs as described herein.

The LNA molecules can include molecules comprising 10-30, e.g., 12-24, e.g., 12, 13, 14, 15, 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the RNA. The LNA molecules can be chemically synthesized using methods known in the art.

The LNA molecules can be designed using any method known in the art; a number of algorithms are known, and are commercially available (e.g., on the internet, for example at exiqon.com). See, e.g., You et al., Nuc. Acids. Res. 34:e60 (2006); McTigue et al., Biochemistry 43:5388-405 (2004); and Levin et al., Nuc. Acids. Res. 34:e142 (2006). For example, “gene walk” methods, similar to those used to design antisense oligos, can be used to optimize the inhibitory activity of the LNA; for example, a series of oligonucleotides of 10-30 nucleotides spanning the length of a target RNA can be prepared, followed by testing for activity. Optionally, gaps, e.g., of 5-10 nucleotides or more, can be left between the LNAs to reduce the number of oligonucleotides synthesized and tested. GC content is preferably between about 30-60%. General guidelines for designing LNAs are known in the art; for example, LNA sequences will bind very tightly to other LNA sequences, so it is preferable to avoid significant complementarity within an LNA. Contiguous runs of more than four LNA residues, should be avoided where possible (for example, it may not be possible with very short (e.g., about 9-10 nt) oligonucleotides). In some embodiments, the LNAs are xylo-LNAs.

For additional information regarding LNAs, see U.S. Pat. Nos. 6,268,490; 6,734,291; 6,770,748; 6,794,499; 7,034,133; 7,053,207; 7,060,809; 7,084,125; and 7,572,582; and U.S. Pre-Grant Pub. Nos. 20100267018: 20100261175; and 20100035968; Koshkin et al. Tetrahedron 54, 3607-3630 (1998); Obika et al. Tetrahedron Lett. 39, 5401-5404 (1998); Jepsen et al., Oligonucleotides 14:130-146 (2004); Kauppinen et al., Drug Disc. Today 2(3):287-290 (2005); and Ponting et al., Cell 136(4):629-641 (2009), and references cited therein.

### Making and Using Inhibitory Nucleic Acids

The nucleic acid sequences used to practice the methods described herein, whether RNA, cDNA, genomic DNA, vectors, viruses or hybrids thereof, can be isolated from a variety of sources, genetically engineered, amplified, and/or expressed/generated recombinantly. Recombinant nucleic acid sequences can be individually isolated or cloned and tested for a desired activity. Any recombinant expression system can be used, including e.g., in vitro, bacterial, fungal, mammalian, yeast, insect or plant cell expression systems.

Nucleic acid sequences of the invention can be inserted into delivery vectors and expressed from transcription units within the vectors. The recombinant vectors can be DNA plasmids or viral vectors. Generation of the vector construct can be accomplished using any suitable genetic engineering techniques well known in the art, including, without limitation, the standard techniques of PCR, oligonucleotide synthesis, restriction endonuclease digestion, ligation, transformation, plasmid purification, and DNA sequencing, for example, as described in Sambrook et al. Molecular Cloning: A Laboratory Manual. (1989)), Coffin et al. (Retroviruses. (1997)) and “RNA Viruses: A Practical Approach” (Alan J. Cann, Ed., Oxford University Press, (2000)). As will be apparent to one of ordinary skill in the art, a variety of suitable vectors are available for transferring nucleic acids of the present disclosure into cells. The selection of an appropriate vector to deliver nucleic acids and optimization of the conditions for insertion of the selected expression vector into the cell, are within the scope of one of ordinary skill in the art without the need for undue experimentation. Viral vectors comprise a nucleotide sequence having sequences for the production of recombinant virus in a packaging cell. Viral vectors expressing nucleic acids of the invention can be constructed based on viral backbones including, but not limited to, a retrovirus, lentivirus, adenovirus, adeno-associated virus, pox virus or alphavirus. The recombinant vectors capable of expressing the nucleic acids of the invention can be delivered as described herein, and persist in target cells (e.g., stable transformants).

Nucleic acid sequences used to practice the methods described here can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896: Narang (1979) Meth. Enzymol. 68:90; Brown (1979) Meth. Enzymol. 68:109; Beaucage (1981) Tetra. Lett. 22:1859; U.S. Pat. No. 4,458,066.

Nucleic acid sequences of the present disclosure can be stabilized against nucleolytic degradation such as by the incorporation of a modification, e.g., a nucleotide modification. For example, nucleic acid sequences of the present disclosure can include a phosphorothioate at least the first, second, or third internucleotide linkage at the 5′ or 3′ end of the nucleotide sequence. As another example, the nucleic acid sequence can include a 2-modified nucleotide, e.g., a 2′-deoxy, 2′-deoxy-2′-fluoro, 2-O-methyl, 2′-O-methoxyethyl (2′-O-MOE), 2′-O-aminopropyl (2-O-AP), 2′-O-dimethylaminoethyl (2′-O-DMAOE), 2′-O-dimethylaminopropyl (2′-O-DMAP), 2-O-dimethylaminoethyloxyethyl (2′-O-DMAEOE), or 2′-O—N-methylacetamido (2′-O—NMA). As another example, the nucleic acid sequence can include at least one 2′-O-methyl-modified nucleotide, and in some embodiments, all of the nucleotides include a 2-O-methyl modification. In some embodiments, the nucleic acids are “locked,” i.e., comprise nucleic acid analogues in which the ribose ring is “locked” by a methylene bridge connecting the 2′-0 atom and the 4′-C atom (see, e.g., Kaupinnen et al., Drug Disc. Today 2(3):287-290 (2005); Koshkin et al., J. Am. Chem. Soc., 120(50):13252-13253 (1998)). For additional modifications see US 20100004320, US 20090298916, and US 20090143326.

Techniques for the manipulation of nucleic acids used to practice the present methods, such as, e.g., subcloning, labeling probes (e.g., random-primer labeling using Klenow polymerase, nick translation, amplification), sequencing, hybridization and the like are well described in the scientific and patent literature, see, e.g., Sambrook et al., Molecular Cloning; A Laboratory Manual 3d ed. (2001); Current Protocols in Molecular Biology, Ausubel et al., eds. (John Wiley & Sons, Inc., New York 2010); Kriegler, Gene Transfer and Expression: A Laboratory Manual (1990); Laboratory Techniques In Biochemistry And Molecular Biology: Hybridization With Nucleic Acid Probes, Part I. Theory and Nucleic Acid Preparation, Tijssen, ed. Elsevier, N.Y (1993).

### Methods of Treatment

### Treating Chronic Kidney Disease

Described herein are methods for treating chronic kidney disease (CKD) and symptoms and disorders associated therewith in a subject, e.g., a mammalian subject, e.g., a human or non-human veterinary subject such as a cat, dog, rabbit, horse, goat, or pig.

Thus, in some embodiments, the present methods can be used for treating kidney fibrosis (e.g., kidney fibrosis associated with CKD) in a subject. Kidney fibrosis can result from various diseases and insults to the kidneys. Examples of such diseases and insults include CKD, metabolic syndrome, vesicoureteral reflux, tubulointerstitial renal fibrosis, diabetes (including diabetic nephropathy), and resultant glomerular nephritis (GN), including, but not limited to, focal segmental glomerulosclerosis and membranous glomerulonephritis, and mesangiocapillary GN. Since kidney fibrosis is associated with loss of blood vessels, this results in secondary ischemia which can also result in glomerular disease with loss of glomerular function. Regardless of the primary cause, insults to the kidneys may result in kidney fibrosis and the concomitant loss of kidney function (Schena, F, and Gesualdo, L., Pathogenic Mechanisms of Diabetic Nephropathy, J. Am. Soc. Nephrol, 16: S30-33 (2005); Whaley-Connell, A., and Sower, J R., Chronic Kidney Disease and the 25 Cardiometabolic Syndrome, J. Clin. Hypert., 8(4): 546-48 (2006)). Conditions associated with kidney fibrosis include, but are not limited to, diabetic nephropathy, chronic kidney disease, systemic lupus erythematosis, vasculitis, IgAnephropathy, other autoimmune diseases, paraprotein diseases, diabetes. Since chronic kidney disease associated with kidney fibrosis is an important risk factor for cardiovascular disease, it would be apparent to a skilled artisan that a therapeutic that prevented or reduced chronic kidney disease and/or kidney fibrosis would have a beneficial effect on cardiac and vascular disease throughout the body. A condition associated with kidney fibrosis, including kidney fibrosis itself can be diagnosed using methods known in the art, e.g., by one or more of a blood test that measures the level of waste products such as creatinine and urea; a urine test that looks for abnormalities: an imaging test using ultrasound to assess kidney's structure and size; and a kidney biopsy.

The methods of the present disclosure can be used for treating CKD and/or conditions associated therewith (e.g., kidney injury and/or kidney fibrosis) in a subject in need thereof. For example, the present methods can be used for treating a subject who has been diagnosed with CKD and/or conditions associated therewith (e.g., kidney fibrosis and/or kidney fibrosis). Additionally, or in the alternative, the present methods can be used for treating a subject who shows one or more symptoms of CKD and/or conditions associated therewith (e.g., kidney fibrosis and/or kidney fibrosis).

CKD can refer to the progressive loss of kidney function over time. In some embodiments, CKD is characterized by hyperphosphatemia (i.e., >4.6 mg/dl) or low glomerular filtration rates (i.e., <90 ml/minute per 1.73 m² of body surface).

However, many CKD patients may have normal serum phosphate levels in conjunction with a sustained reduction in glomerular filtration rate (GFR) for 3 or more months, or a normal GFR in conjunction with sustained evidence of a structural abnormality of the kidney. In some embodiments, a subject with CKD can be a subject with a sustained reduction in GFR<60 mi/min per 1.73 m² of body surface for 3 or more months; and/or a structural or functional abnormality of renal function for 3 or more months even in the absence of a reduced GFR. Structural or anatomical abnormalities of the kidney could be defined as but not limited to persistent microalbuminuria or proteinuria or hematuria or presence of renal cysts. Common symptoms of CKD include tiredness, nausea, urine-like odor to the breath, bone pain, abnormally dark or light skin, itching, restless leg syndrome, blood in stools, bruising easily, pedal edema, and peripheral edema. CKD can be diagnosed through, e.g., one or more of medical history; a blood test that measures complete blood count, BUN level, or creatinine level; renal flow and scan; and renal ultrasound. Kidney damage, e.g., kidney damage associate with CKD, refers to pathologic abnormalities, and can be diagnosed or detected by imaging studies or renal biopsy, abnormalities in urinary sediment, and/or increased urinary albumin excretion rates.

The 2012 KDIGO CKD classification recommends details about the cause of the CKD and classifies it into 6 categories based on glomerular filtration rate (G1 to G5 with G3 split into 3a and 3b). It also includes the staging based on three levels of albuminuria (A1. A2, and A3), with each stage of CKD being sub-categorized according to the urinary albumin-creatinine ratio in (mg/gm) or (mg/mmol) in an early morning “spot” urine sample (Vaidya S R, Aeddula N R. Chronic Kidney Disease. [Updated 2022 Oct 24]. In: StatPearls [Internet]. Treasure Island (FL): StatPearls Publishing; 2024, Available from: ncbi.nlm.nih.gov/books/NBK535404/; Inker et al. KDOQI US commentary on the 2012 KDIGO clinical practice guideline for the evaluation and management of CKD. Am J Kidney Dis. 2014 May, 63(5):713-35). The 6 categories include: stage 1 or G1 (GFR 90 ml/min per 1.73 m² and above); stage 2 or G2 (GFR 60 to 89 ml/min per 1.73 m²); stage 3a or G3a (GFR 45 to 59 ml/min per 1.73 m²): stage 3b or G3b (GFR 30 to 44 ml/min per 1.73 m²); stage 4 or G4 (GFR 15 to 29 ml/min per 1.73 m²); and stage 5 or G5 (GFR less than 15 ml/min per 1.73 m² or treatment by dialysis). Stratification also comprises three categories of albuminuria, where patients are stratified based on albumin to creatinine ratio (ACR). The three levels of albuminuria include:A1 with ACR less than 30 mg/gm (less than 3.4 mg/mmol): A2 with ACR 30 to 299 mg/gm (3.4 to 34 mg/mmol); and A3 with ACR greater than 300 mg/gm (greater than 34 mg/mmol).

In some embodiments, the present methods are used for treating: a subject who has stage 1 CKD or G1 CKD (e.g., with GFR 90 ml/min per 1.73 m² and above); a subject who has stage 2 CKD or G2 CKD (e.g., with GFR 60 to 89 ml/min per 1.73 m2): a subject who has stage 3a CKD or G3a CKD (e.g., with GFR 45 to 59 ml/min per 1.73 m²); a subject who has stage 3b CKD or G3b CKD (e.g., with GFR 30 to 44 ml/min per 1.73 m²); a subject who has stage 4 CKD or G4 CKD (e.g., with GFR 15 to 29 ml/min per 1.73 m²); and/or a subject who has stage 5 CKD or and G5 CKD (e.g., with GFR less than 15 ml/min per 1.73 m²). In some embodiments, the present methods are used for treating: a subject who has A1 CKD (e.g., with ACR less than 30 mg/gm (less than 3.4 mg/mmol)); a subject who has A2 CKD (e.g., with ACR 30 to 299 mg/gm (3.4 to 34 mg/mmol)); and/or a subject who has A3 CKD (e.g., with ACR greater than 300 mg/gm (greater than 34 mg/mmol)).

In some embodiments, the present methods are used for treating a subject who has (e.g., is diagnosed with and/or shows symptoms of) early-stage or intermediate-stage CKD. Early-stage CKD can refer to one or more of stage 1 CKD or G1 CKD (e.g., with GFR 90 ml/min per 1.73 m² and above); stage 2 CKD or G2 CKD (e.g., with GFR 60 to 89 ml/min per 1.73 m²); and A1 CKD (e.g., with ACR less than 30 mg/gm (less than 3.4 mg/mmol)). Intermediate-stage CKD can refer to one or more of stage 3a CKD or G3a CKD (e.g., with GFR 45 to 59 ml/min per 1.73 m²); stage 3b CKD or G3b CKD (e.g., with GFR 30 to 44 ml/min per 1.73 m²); and A2 CKD (e.g., with ACR 30 to 299 mg/gm (3.4 to 34 mg/mmol)). Thus, the present methods can be used for treating: a subject who has (e.g., is diagnosed with and/or shows symptoms of) stage 1 CKD or G1 CKD (e.g., with GFR 90 ml/min per 1.73 m² and above); a subject who has (e.g., is diagnosed with and/or shows symptoms of) stage 2 CKD or G2 CKD (e.g., with GFR 60 to 89 ml/min per 1.73 m²); a subject who has (e.g., is diagnosed with and/or shows symptoms of) A1 CKD (e.g., with ACR less than 30 mg/gm (less than 3.4 mg/mmol)); a subject who has (e.g., is diagnosed with and/or shows symptoms of) stage 3a CKD or G3a CKD (e.g., with GFR 45 to 59 ml/min per 1.73 m²); a subject who has (e.g., is diagnosed with and/or shows symptoms of) stage 3b CKD or G3b CKD (e.g., with GFR 30 to 44 ml/min per 1.73 m²); and/or a subject who has (e.g., is diagnosed with and/or shows symptoms of) A2 CKD (e.g., with ACR 30 to 299 mg/gm (3.4 to 34 mg/mmol)).

In some embodiments, a subject who is treated by the present methods does not have late-stage chronic kidney disease. Late-stage CKD can refer to one or more of: stage 4 CKD or G4 CKD (e.g., with GFR 15 to 29 ml/min per 1.73 m²); stage 5 CKD or G5 CKD (e.g., with GFR less than 15 ml/min per 1.73 m² or treatment by dialysis); and A3 CKD (e.g., with ACR greater than 300 mg/gm (greater than 34 mg/mmol)). Thus, the present methods can be used for treating: a subject who does not have (e.g., is not diagnosed with and/or does not show symptoms of) stage 4 CKD or G4 CKD (e.g.. GFR 15 to 29 mil/min per 1.73 m²); a subject who does not have (e.g., is not diagnosed with and/or does not show symptoms of) stage 5 CKD or G5 CKD (e.g., GFR less than 15 ml/min per 1.73 m² or treatment by dialysis); and/or a subject who does not have (e.g., is not diagnosed with and/or does not show symptoms of) A3 CKD (e.g.. ACR greater than 300 mg/gm (greater than 34 mg/mmol)).

In some embodiments, the present methods are used for treating a subject who has been exposed to one or more environmental toxins associated with CKD. Such environmental toxins can include, without limitation, heavy metals (e.g., lead, cadmium, arsenic, mercury, and/or uranium) arsine gas, carbon tetrachloride, methylene chloride, toluene, and trichloroethylene. In some embodiments, a subject who is treated by the present methods does not have chronic kidney disease-mineral and bone disorder (CKD-MBD). In some embodiments, a subject who is treated by the present methods does not have acute kidney injury (AKI).

In some embodiments, a subject is diagnosed with CKD and/or conditions associated therewith (e.g., kidney injury and/or kidney fibrosis) by assessment of renal function. Renal function can be assessed by methods known in the art, e.g., by one or more of: assessment of GFR; assessment of serum creatinine; assessment of Blood Urea Nitrogen (BUN); assessment of cystatin C; assessment for albuminuria; assessment for proteinuria; assessment of tubular function; urine analysis (e.g., assessment of urine characteristics); and assessment of one or more biomarkers. In certain embodiments, a subject is diagnosed as having CKD and/or conditions associated therewith (e.g., kidney injury and/or kidney fibrosis), when the subject has increased expression of one or more markers of renal dysfunction (e.g., one or more of creatinine, BUN, albuminuria, proteinuria, and cystatin C), as compared to a heathy control (e.g., an individual who is not diagnosed with CKD and/or conditions associated therewith). In certain embodiments, a subject is diagnosed as having CKD and/or conditions associated therewith (e.g., kidney injury and/or kidney fibrosis), when the subject has reduced or deteriorated renal function, as compared to a heathy control (e.g., an individual who is not diagnosed with CKD and/or conditions associated therewith).

In certain embodiments, a subject is diagnosed as having CKD and/or conditions associated therewith (e.g., kidney injury and/or kidney fibrosis), when the subject has increased expression of one or more markers of fibrosis (e.g., one or more of alpha-smooth muscle actin (αSMA), Collal, and Vimentin (Vim)), as compared to a heathy control (e.g., an individual who is not diagnosed with CKD and/or conditions associated therewith).

In certain embodiments, a subject is diagnosed as having CKD and/or conditions associated therewith (e.g., kidney injury and/or kidney fibrosis), when the subject has increased expression of one or more injury markers, such as renal injury markers (e.g., one or more of Col3al, Vim, Serpinel, Pai-1, Fibronectin-1, and Col8al), as compared to a heathy control (e.g., an individual who is not diagnosed with CKD and/or conditions associated therewith).

In certain embodiments, a subject is diagnosed as having CKD and/or conditions associated therewith (e.g., kidney injury and/or kidney fibrosis), when the subject has increased proximal tubule injury, as compared to a heathy control (e.g., an individual who is not diagnosed with CKD and/or conditions associated therewith).

Proximal tubule injury can be assessed by one or more of assessment for expression of one or more proximal tubule injury markers (e.g., p21); assessment for expression of one or more markers of intact proximal epithelial cells (e.g., one or more of Lrp2, Atplal, Slc34al, Hnf4a, and Slc17a2): assessment of epithelial cell integrity; and assessment of number of healthy proximal tubule cells. Accordingly, in certain embodiments, a subject is diagnosed as having CKD and/or conditions associated therewith (e.g., kidney injury and/or kidney fibrosis), when, as compared to a heathy control (e.g., an individual who is not diagnosed with CKD and/or conditions associated therewith), the subject has one or more of the following: increased expression of one or more proximal tubule injury markers (e.g., p21); reduced expression of one or more markers of intact proximal epithelial cells (e.g., one or more of Lrp2, Atplal. Slc34al, Hnf4a, and Slc17a2): reduced epithelial cell integrity; and reduced number of healthy proximal tubule cells.

Methods of the present disclosure can include administering a therapeutically effective amount of one or more SIK inhibitor(s), as described herein, to a subject who is in need of, or who has been determined to be in need of, such treatment. Treatment by the present methods can ameliorate at least one symptom of CKD and/or conditions associated therewith (e.g., kidney injury and/or kidney fibrosis) in a subject, as compared to before treatment (or as compared to a subject with CKD and/or conditions associated therewith who has not been treated by the present methods). Often, CKD and/or conditions associated therewith (e.g., kidney injury and/or kidney fibrosis) results in increased levels of BUN or creatinine, hyperphosphatemia and/or low glomerular filtration rates; thus, treatment by the present methods can result in a reduction in BUN, phosphate, or creatinine levels, and a return or approach to normal kidney function (e.g., glomerular filtration rates of at least 90 ml/minute per 1.73 m² of body surface), as compared to before treatment (e.g., before the SIK inhibitor(s) is administered to the subject) or as compared to a subject with CKD and/or conditions associated therewith who has not been treated by the present methods.

In some embodiments, treatment by the present method (e.g., administration of a therapeutically effective amount of one or more SIK inhibitors) results in decreased kidney fibrosis (e.g., as detectable on ultrasound) in a subject, as compared to before treatment (e.g., before the SIK inhibitor(s) is administered to the subject) or as compared to a subject with CKD, kidney fibrosis, kidney injury, and/or conditions associated therewith who has not been treated by the present methods.

In some embodiments, treatment by the present method (e.g., administration of a therapeutically effective amount of one or more SIK inhibitors) results in decreased kidney injury (e.g., as detectable on ultrasound and/or by one or more renal injury markers) in a subject, as compared to before treatment (e.g., before the SIK inhibitor(s) is administered to the subject) or as compared to a subject with CKD, kidney fibrosis, kidney injury, and/or conditions associated therewith who has not been treated by the present methods.

In some embodiments, treatment of a subject by the present method (e.g., administration of a therapeutically effective amount of one or more SIK inhibitors) results in reduced expression of one or more markers of renal dysfunction (e.g., one or more of creatinine, BUN, proteinuria, and cystatin C) in the subject, as compared to before treatment (e.g., before the SIK inhibitor(s) is administered to the subject) or as compared to a subject with CKD, kidney fibrosis, kidney injury, and/or conditions associated therewith who has not been treated by the present methods.

In some embodiments, treatment of a subject by the present method (e.g., administration of a therapeutically effective amount of one or more SIK inhibitors) results in reduced expression of one or more fibrosis markers (e.g., one or more of alpha-smooth muscle actin (αSMA), Collal, and Vimentin) in the subject, as compared to before treatment (e.g., before the SIK inhibitor(s) is administered to the subject) or as compared to a subject with CKD, kidney fibrosis, kidney injury, and/or conditions associated therewith who has not been treated by the present methods.

In some embodiments, treatment of a subject by the present method (e.g., administration of a therapeutically effective amount of one or more SIK inhibitors) results in reduced expression of one or more injury markers, such as renal injury markers (e.g., one or more of Col3a1, Vim, Serpinel, Pai-1, Fibronectin-1, and Col8a1) in the subject, as compared to before treatment (e.g., before the SIK inhibitor(s) is administered to the subject) or as compared to a subject with CKD, kidney fibrosis, kidney injury, and/or conditions associated therewith who has not been treated by the present methods.

In some embodiments, treatment of a subject by the present method (e.g., administration of a therapeutically effective amount of one or more SIK inhibitors) results in reduced expression of one or more proximal tubule injury markers (e.g., p21) in the subject, as compared to before treatment (e.g., before the SIK inhibitor(s) is administered to the subject) or as compared to a subject with CKD, kidney fibrosis, kidney injury, and/or conditions associated therewith who has not been treated by the present methods.

In some embodiments, treatment of a subject by the present method (e.g., administration of a therapeutically effective amount of one or more SIK inhibitors) results in increased expression of one or more markers of intact proximal epithelial cells (e.g., one or more of Lrp2, Atplal, Slc34al, Hnf4a, and Slc17a2) in the subject, as compared to before treatment (e.g., before the SIK inhibitor(s) is administered to the subject) or as compared to a subject with CKD, kidney fibrosis, kidney injury, and/or conditions associated therewith who has not been treated by the present methods.

In some embodiments, treatment of a subject by the present method (e.g., administration of a therapeutically effective amount of one or more SIK inhibitors) results in preservation of epithelial cell integrity in the subject, as compared to before treatment (e.g., before the SIK inhibitor(s) is administered to the subject) or as compared to a subject with CKD, kidney fibrosis, kidney injury, and/or conditions associated therewith who has not been treated by the present methods.

In some embodiments, treatment of a subject by the present method (e.g., administration of a therapeutically effective amount of one or more SIK inhibitors) results in increased number of healthy proximal tubule cells in the subject, as compared to before treatment (e.g., before the SIK inhibitor(s) is administered to the subject) or as compared to a subject with CKD, kidney fibrosis, kidney injury, and/or conditions associated therewith who has not been treated by the present methods.

In some embodiments, treatment of a subject by the present method (e.g., administration of a therapeutically effective amount of one or more SIK inhibitors) results in improved renal function in the subject, as compared to before treatment (e.g., before the SIK inhibitor(s) is administered to the subject) or as compared to a subject with CKD, kidney fibrosis, kidney injury, and/or conditions associated therewith who has not been treated by the present methods.

### Preventing Chronic Kidney Disease

The methods of the present disclosure can be used for preventing (e.g., reducing the risk of occurrence or delaying the occurrence of) chronic kidney disease (CKD) and/or conditions associated therewith (e.g., kidney injury and/or kidney fibrosis) in a subject in need thereof. For example, the present methods can be useful for reducing the risk of occurrence or delaying the occurrence of CKD, kidney fibrosis, kidney injury, and/or conditions associated therewith in a subject who is at risk for developing CKD, kidney fibrosis, kidney injury, and/or conditions associated therewith. Risk factors for CKD include, but are not limited to, diabetes, high blood pressure, heart disease, obesity, family history of CKD, inherited kidney disorders, past damage to the kidneys, and older age. Thus, in some embodiments, the present methods can be used for reducing the risk of occurrence or delaying the occurrence of CKD and/or conditions associated therewith (e.g., kidney injury and/or kidney fibrosis) in a subject who has diabetes, high blood pressure, heart disease, obesity, family history of CKD, inherited kidney disorders, past damage to the kidneys, and/or older age. Risk factors for CKD can also include exposure to one or more environmental toxins, such as, one or more of heavy metals (e.g., lead, cadmium, arsenic, mercury, and/or uranium) arsine gas, carbon tetrachloride, methylene chloride, toluene, and trichloroethylene. Thus, in some embodiments, the present methods can be used for reducing the risk of occurrence or delaying the occurrence of CKD and/or conditions associated therewith (e.g., kidney injury and/or kidney fibrosis) in a subject who has been exposed to one or more of such environmental toxins.

Treatment of a subject by the present method (e.g., administration of a therapeutically effective amount of one or more SIK inhibitors) can reduce the risk of occurrence or delay the occurrence of CKD, kidney fibrosis, and/or kidney injury in the subject, as compared to a subject who is at risk for developing CKD, kidney fibrosis, and/or kidney injury, but has not been treated by the present methods.

Dosage An “effective amount” is an amount sufficient to effect beneficial or desired results. Thus, a therapeutic amount (or a therapeutically effective amount) is one that achieves the desired therapeutic effect. For example, a therapeutic amount (or a therapeutically effective amount) of SIK inhibitor(s) described herein can be the amount that when administered to a subject in need thereof, ameliorates at least one symptom of CKD and/or conditions associated therewith (e.g., kidney injury and/or kidney fibrosis) in the subject, as compared to before treatment (or as compared to a subject with CKD and/or conditions associated therewith who has not been treated by the present methods). This amount can be the same or different from a prophylactically effective amount, which is an amount necessary to prevent onset of disease or disease symptoms. For example, a therapeutically effective amount of SIK inhibitor(s) described herein can be the amount that when administered to a subject in need thereof, reduces the risk of occurrence or delays the occurrence of CKD, kidney fibrosis and/or kidney injury in the subject, as compared to a subject who is at risk for developing CKD, kidney fibrosis and/or kidney injury, but who has not been treated by the present methods.

An effective amount can be administered in one or more administrations, applications or dosages. A therapeutically effective amount of a SIK inhibitor depends on the particular inhibitor selected. Pharmaceutical formulations comprising the SIK inhibitor(s) can be administered one or more times per day, one or more times per week (including once every other day), or one or more times per month. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the SIK inhibitor(s) described herein can include a single treatment or a series of treatments. For example, pharmaceutical formulations comprising the SIK inhibitor(s) can be administered to a subject one or more times (e.g., twice, thrice, four times, five times, six times, seven times, eight times, or more) at hourly, daily, weekly, or monthly intervals.

Dosage, toxicity and therapeutic efficacy of the SIK inhibitor(s) can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. SIK inhibitors which exhibit high therapeutic indices are preferred. While SIK inhibitors that exhibit toxic side effects may be used, care should be taken to design a delivery system that targets such SIK inhibitors to the site of affected tissue in order to minimize potential damage to uninfected cells and, thereby, reduce side effects.

The data obtained from cell culture assays and animal studies can be used in formulating a range of dosage for use in humans. The dosage of such SIK inhibitors lies preferably within a range of circulating concentrations that include the ED50 with little or no toxicity. The dosage may vary within this range depending upon the dosage form employed and the route of administration utilized. For any SIK inhibitor used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Pharmaceutical Compositions

When employed as pharmaceuticals, the compounds and salts provided herein can be administered in the form of pharmaceutical compositions. Additionally, or in the alternative, antibodies or inhibitory nucleic acid sequences described herein can also be administered in the form of pharmaceutical compositions.

These compositions can be prepared as described herein or elsewhere (see, e.g., Remington: The Science and Practice of Pharmacy, 21st ed. 2005), and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insuffilation of powders or aerosols, including by nebulizer; intratracheal or intranasal), oral, or parenteral. Parenteral administration includes intravenous, intraarterial, subcutaneous, intraperitoneal intramuscular or injection or infusion; or intracranial. (e.g., intrathecal or intraventricular, administration). Parenteral administration can be in the form of a single bolus dose, or may be, for example, by a continuous perfusion pump. In some embodiments, the compounds, salts, and pharmaceutical compositions provided herein are suitable for parenteral administration. In some embodiments, the inhibitory compounds, salts, antibodies, inhibitory nucleic acid sequences, and/or pharmaceutical compositions provided herein are suitable for intravenous administration. In some embodiments, the inhibitory compounds, salts, antibodies, inhibitory nucleic acid sequences, and/or pharmaceutical compositions provided herein are suitable for oral administration.

Also provided are pharmaceutical compositions which contain, as the active ingredient (or active agent), an inhibitory compound described herein, or a pharmaceutically acceptable salt thereof, in combination with one or more non-toxic pharmaceutically acceptable carriers (e.g., excipients). Additionally, or in the alternative, provided herein are pharmaceutical compositions which contain, as the active ingredient (or active agent), an antibody or inhibitory nucleic acid described here, in combination with one or more non-toxic pharmaceutically acceptable carriers (e.g., excipients). In making the compositions provided herein, the active ingredient can be typically mixed with an excipient, diluted by an excipient or enclosed within such a carrier in the form of, for example, a capsule, sachet, paper, or other container.

When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders.

Some examples of suitable excipients include, without limitation, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include, without limitation, lubricating agents such as talc, magnesium stearate, and mineral oil; wetting agents; emulsifiers and lubricants, such as sodium lauryl sulfate and magnesium stearate; suspending agents; preserving agents, such as methyl- and propylhydroxy-benzoates; sweetening agents; flavoring agents, or combinations thereof.

The active ingredient can be effective over a wide dosage range and is generally administered in a therapeutically effective amount. It will be understood, however, that the amount of the active ingredient (e.g., inhibitory compound, antibody, and/or inhibitory nucleic acid described herein) actually administered will usually be determined by a physician, according to the relevant circumstances, including the condition to be treated, the chosen route of administration, the actual compound administered, the age, weight, and response of the individual subject, the severity of the subject's symptoms, and the like.

Formulations of the compositions of the present disclosure can include those suitable for intradermal, inhalation, oral/nasal, topical, parenteral, rectal, and/or intravaginal administration. The formulations may conveniently be presented in unit dosage form and may be prepared by any methods well known in the art of pharmacy.

The amount of active ingredient (e.g., inhibitory compound, antibody, and/or inhibitory nucleic acid described herein) which can be combined with a carrier material to produce a single dosage form will vary depending upon the host being treated, the particular mode of administration, e.g., oral, intravenous, subcutaneous, intradermal, or inhalation. The amount of active ingredient which can be combined with a carrier material to produce a single dosage form will generally be that amount of the active ingredient which produces a therapeutic effect.

Pharmaceutical formulations for oral administration can be formulated using active ingredients (e.g., inhibitory compound, antibody, and/or inhibitory nucleic acid described herein) and pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional non-active ingredients, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active ingredient (e.g., inhibitory compound, antibody, and/or inhibitory nucleic acid described herein) in admixture with excipients suitable for the manufacture of aqueous suspensions, e.g., for aqueous intradermal injections. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., poly oxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

In some embodiments, oil-based pharmaceuticals are used for administration of inhibitors of the present disclosure (e.g., inhibitory compound, antibody, and/or inhibitory nucleic acid described herein). Oil-based suspensions can be formulated by suspending an active agent in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102.

Pharmaceutical formulations can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. In alternative embodiments, these injectable oil-in-water emulsions of the invention comprise a paraffin oil, a sorbitan monooleate, an ethoxylated sorbitan monooleate and/or an ethoxylated sorbitan trioleate.

The pharmaceutical formulations of the present disclosure can also be administered by intranasal, intraocular and intravaginal routes including suppositories, insufflation, powders and aerosol formulations (for examples of steroid inhalants, see e.g., Rohatagi (1995) J. Clin. Pharmacol. 35:1187-1193; Tjwa (1995) Ann. Allergy Asthma Immunol. 75:107-111). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

The pharmaceutical formulations of the present disclosure can also be delivered transdermally, by a topical route, formulated as applicator sticks, solutions, suspensions, emulsions, gels, creams, ointments, pastes, jellies, paints, powders, and aerosols.

The pharmaceutical formulations of the present disclosure can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In some embodiments, pharmaceutical formulations of the present disclosure are parenterally administered, such as by intravenous (IV) administration or administration into a body cavity or lumen of an organ. These formulations can comprise an active agent (e.g., inhibitory compound, antibody, and/or inhibitory nucleic acid described herein) dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose, any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

In some embodiments, the pharmaceutical compositions and formulations are lyophilized. Stable lyophilized formulations comprising an active ingredient (e.g., inhibitory compound, antibody, and/or inhibitory nucleic acid described herein) can be made by lyophilizing a solution comprising a pharmaceutical formulation of the invention and a bulking agent, e.g., mannitol, trehalose, raffinose, and sucrose or mixtures thereof. A process for preparing a stable lyophilized formulation can include lyophilizing a solution of about 2.5 mg/mL protein, about 15 mg/mL sucrose, about 19 mg/mL NaCl, and a sodium citrate buffer having a pH greater than 5.5 but less than 6.5. See, e.g.. U.S. 20040028670.

The compositions and formulations can be delivered by the use of liposomes. By using liposomes, particularly where the liposome surface carries ligands specific for target cells, or are otherwise preferentially directed to a specific organ, one can focus the delivery of the active agent into target cells in vivo. See, e.g.. U.S. Pat. Nos. 6,063,400: 6,007,839: A1-Muhammed (1996) J. Microencapsul. 13:293-306: Chonn (1995) Curr. Opin. Biotechnol. 6:698-708; Ostro (1989) Am. J. Hosp. Pharm. 46:1576-1587. As used in the present invention, the term “liposome” means a vesicle composed of amphiphilic lipids arranged in a bilayer or bilayers. Liposomes are unilamellar or multilamellar vesicles that have a membrane formed from a lipophilic material and an aqueous interior that contains the composition to be delivered.

Cationic liposomes are positively charged liposomes that are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes can also include “sterically stabilized” liposomes, i.e., liposomes comprising one or more specialized lipids. When incorporated into liposomes, these specialized lipids result in liposomes with enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

The formulations of the present disclosure can be administered for prophylactic and/or therapeutic treatments. In some embodiments, for therapeutic applications, pharmaceutical formulations of the present disclosure are administered to a subject who is in need of treatment for chronic kidney disease (CKD) and/or kidney fibrosis, in an amount sufficient to cure, alleviate or partially arrest the clinical manifestations of CKD, kidney fibrosis, and/or complications thereof; this can be called a therapeutically effective amount. For example, in some embodiments, pharmaceutical compositions of the present disclosure are administered in an amount sufficient to result in one or more of reduced expression of one or more markers of renal dysfunction in the subject; reduced expression of one or more fibrosis markers in the subject; reduced expression of one or more renal injury markers in the subject: reduced expression of one or more proximal tubule injury markers in the subject; increased expression of one or more markers of intact proximal epithelial cells in the subject: preservation of epithelial cell integrity in the subject; increased number of healthy proximal tubule cells in the subject; and improved renal function in the subject. Additionally, or in the alternative, for prophylactic applications, pharmaceutical formulations of the present disclosure can be administered to a subject who is at risk for developing CKD and/or kidney fibrosis, in an amount sufficient to reduce the risk of occurrence or delaying the occurrence of CKD, kidney fibrosis, and/or complications thereof; and this can be called a therapeutically effective amount. For example, in some embodiments, pharmaceutical compositions of the present disclosure are administered in an amount sufficient to reduce the risk of occurrence or delaying the occurrence of CKD, kidney fibrosis, and/or complications thereof in a subject.

The amount of pharmaceutical composition adequate to accomplish this is a therapeutically effective dose. The dosage schedule and amounts effective for this use, i.e., the dosing regimen, will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; Remington: The Science and Practice of Pharmacy, 21st ed., 2005). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods of the invention are correct and appropriate.

Single or multiple administrations of formulations can be given depending on for example: the dosage and frequency as required and tolerated by the patient, the degree and amount of therapeutic effect generated after each administration (e.g., effect on expression of one or more fibrosis markers in the subject; effect on expression of one or more injury markers in the subject: effect on expression of one or more proximal tubule injury markers in the subject; effect on expression of one or more markers of intact proximal epithelial cells in the subject; effect on epithelial cell integrity in the subject; effect on number of healthy proximal tubule cells in the subject; and effect on renal function in the subject), and the like. The formulations should provide a sufficient quantity of active agent to effectively treat, prevent or ameliorate conditions, diseases or symptoms. In some embodiments, pharmaceutical formulations for oral administration are in a daily amount of between about 1 to 100 or more mg per kilogram of body weight per day. Lower dosages can be used, in contrast to administration orally, into the blood stream, into a body cavity or into a lumen of an organ. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington: The Science and Practice of Pharmacy, 21st ed., 2005.

### Combination Treatments

The methods described herein can include the use of standard treatments in addition to the SIK inhibitor(s). Treatments for CKD and/or conditions associated therewith (e.g., kidney injury and/or kidney fibrosis) are known in the art and include, by way of non-limiting example, dialysis; transplant; low protein diet; one or more ACE inhibitors (e.g., one or more of perindopril, captopril, enalapril, lisinopril, and ramipril): one or more angiotensin II receptor blockers (ARBs) (e.g., one or more of Losartan, irbesartan, olmesartan, candesartan, valsartan,fimasartan, and telnisartan); one or more lipid control agents (e.g., statins): one or more D-vitamin supplementation; one or more phosphate control agents; one or more anemia control agents (e.g., erythroid stimulating agents); one or more acidosis prevention agents (e.g., sodium bicarbonate); and/or one or more uric acid control agents (e.g., allopurinol).

### EXAMPLES

The invention is further described in the following examples, which do not limit the scope of the invention described in the claims.

### Example 1. SIK Inhibitors to Treat Chronic Kidney Disease

Here we show that pharmacologic or genetic inhibition of salt inducible kinase (SIK)2/SIK3 ameliorates epithelial cell injury in complementary mouse and human chronic kidney disease models. In particular, in the studies described below, SK-124 was used for pharmacologic inhibition of SIK. SIK-124 is a selective SIK2/SIK3 inhibitor that is described in detail, e.g., at WO 2022/031928, Sato T et al. (Proc Natl Acad Sci USA. 2022 Dec. 13; 119(50):e2214396119), and Yoon S H et al. (J Clin Invest. 2023;133(9):e163627), each of which is incorporated herein by reference in its entirety.

### Materials and Methods

### Compounds

Tamoxifen (Sigma, T5648) for Ubiquitin-Cre^{ERt2}; Sik2^{ff}; Sik3^{ff} mice was prepared in sunflower oil to inject 1 mg of tamoxifen per mouse. SK-124 (6, 7) was prepared in vehicle (15% hydroxy-propyl beta-cyclodextrin (Sigma, H107) dissolved in sterile water) for in vivo use and dissolved in DMSO at 10 mM for in vitro use. Human PTH1-34 was dissolved in 10 mM acetic acid, then further diluted in 0.1% BSA for in vitro use or in PTH vehicle (10 mM citric acid, 150 mM NaCl, 0.05% Tween-80, pH 5) for in vivo use. 1,25-dihydroxy-vitamin D (HY-10002, MedChemExpress) was reconstituted in ethanol and cisplatin (Sigma, 232120) was dissolved in PBS to treat the human pluripotent cell derived kidney organoids. For the experiments in kidney Glil cells, recombinant human TGF-beta (Bio-Techne R+D Systems, 7754-BH-025) was reconstituted in 4 mM HCl with 0.1% BSA.

### Mice

7-week-old female CD1 mice were obtained from Charles River Laboratories (Wilmington, MA) and were fed on 0.2% adenine containing diet (TD.130900; 0.2% Adenine, 0.9% Phosphate, 0.5% Calcium; Envigo) or control diet (TD. 96348) for 6 or 8 weeks to induce chronic kidney disease (CKD). Mice were oral gavaged with 40 mg/kg SK-124, or intraperitonially injected with 80 μg/kg PTH, or treated with vehicle 5 days a week for the duration of the special diet.

For studies with Ubiquitin-Cre^{ERt2}; Sik2^{ff}; Sik3^{ff} mice (JAX #007001 for Ubiquitin-Cre^{ERt2}: RRID:MGL:5905012 for Sik2 foxed mice; and Sik3^{tmla}(EUCOMM)Hmgu mice (RRID:MGI:5085429) bred to PGK1 FLPo mice (JAX #011065) in order to generate Sik3 foxed mice), tamoxifen was injected (1 mg, IP, Q48 hours, 3 injections total) in all animals including Cre-negative control littermates housed in cages with Pure-o′Cel bedding at 5 weeks of age. Mice were then fed on 0.2% adenine diet from 6 weeks to 9 weeks of age. Mice were fasted for 6 hours and treated with SK-124 and PTH 4 hours and 2 hours before the sacrifice. Blood and spot urine samples were collected by cheek pouch bleeding and by pipetting urine collected on hydrophobic sand (Labsand; Braintree, MA). Kidneys were immediately snap-frozen in liquid nitrogen for TRIzol (Life Technologies)-Chloroform (Fischer Scientific) based RNA isolation or protein isolation, or fixed in 10% formalin at 4C for 24 hrs for histology. All serum and urine measurements were performed using commercial bioassay detection reagents listed in Table 1, following the manufacturers' instructions. Urine protein levels were measured by Bradford assay and normalized to urine creatinine.

TABLE 1
Bioassay detection reagents

| Analyte | Manufacturer | Cat Number |
|---|---|---|
| Phosphate | Abcam | ab65622 |
| Creatinine | Stanbio | 0430-120 |
| Urea Nitrogen Direct | Stanbio | 0580-250 |
| Cystatin C | R&D Systems | MSCTC0 |
| 1,25-(OH)2-vitamin D | IDS | AC-62F1 |
| intact PTH | Immunotopics | 60-2305 |
| intact FGF23 | Immunotopics | 60-6800 |

### Human Kidney Organoids

BJFF human induced pluripotent stem cells (hPSCs) were maintained on Geltrexcoated (Thermo Fisher Scientific) plates using StemFit Basic04 Complete (Ajinomoto Co. Inc.) as previously reported(25). The cells were passaged weekly using Accutase (STEMCELL Technologies) for dissociation and Y27632 (Tocris) for adhesion. Directed differentiation of hPSCs into kidney organoids has been published elsewhere (29). Briefly, hPSCs were differentiated into metanephric mesenchyme cells on day 8 by a three-step directed differentiation protocol. Metanephric mesenchyme was transferred into suspension culture in 96-well ultralow adhesion plates (Coming) and further differentiated into kidney organoids, as previously reported(25). Quality control measures of SIX2 immunostaining on day 8 of differentiation and CDH1, LTL, PODXL, CD146, MEIS1/2/3, and SYTOX-blue staining on day 21 were performed on all batches of kidney organoids to limit batch-to-batch heterogeneity. Kidney organoids were maintained in 200 μl of basal media consisting of Advanced RPMI (Thermo Fisher Scientific) and GlutaMAX (Thermo Fisher Scientific) in 96-well plates, as previously reported. Media changes were conducted three times weekly with removal of 90 [l and addition of 100 μl of fresh basal media.

For cisplatin induced injury, day 49 organoids were treated with 5 μM cisplatin in basal media after full media change. After 24 hours, full media exchanges were performed. This 24-hour treatment was performed twice a week, on day 52-53, day 56-57, day 59-60, and day 63-64 as well, for a total of five cisplatin treatments. Samples were obtained for RT-qPCR and immunostaining immediately following each 24-hour treatment with cisplatin, and from corresponding controls.

Visualization of nephron structures and p21 and γH2AX expression on kidney organoids

Following the treatment, the organoids were collected and immediately fixed in 4% PFA in PBS for 1 hour at room temperature, submerged in 30% sucrose overnight at 4° C., then embedded in O.C.T. Compound (4585. Fisher HealthCare). Subsequently, 10-pm frozen sections were prepared via cryo-sectioning by a cryostat. After 1 hr blocking at room temperature, the sections were treated overnight at 4° C. with primary antibodies in antibody dilution buffer (ADB) with biotinylated LTL (1:200, B-1325, Vector), rat anti-CDH1 (1:500, ab11512, Abcam), goat anti-PODXL (1:500, AF1658, R&D Systems), as well as the antibody of interest, either mouse anti-p21 (1:200, NBP2-43697, Novus Biologicals), or rabbit anti-γH2AX (1:100, 2577, Cell Signaling) antibodies. For the optimum labeling of LTL by biotin/streptavidin interaction, the Streptavidin/Biotin Blocking Kit (SP-2002, Vector Laboratories) was applied according to the manufacturer's instructions. After washing, the samples were stained for 1 hour at RT with the nuclear dye-SYTOX Blue (1:500. S 11348, Invitrogen) and the corresponding secondary antibodies in ADB such as streptavidin-AF750 (1:500, S21384, Invitrogen), donkey anti-rat IgG-AF594 (1:500, A21209, Invitrogen), donkey anti-goat IgG-AF555 (1:500, A21432, Invitrogen), and donkey anti-rabbit IgG-AF488 (1:500, A21206, Invitrogen) for γH2AX or donkey anti-mouse IgG-AF488 (1:500, A21202, Invitrogen) for p21 antibodies as well as mouse anti-Meisl/⅔ (1:200, sc-101850, SantaCruz) conjugated antibody, and slides were mounted with Vectashield antifade mounting medium (H1000, Vector Laboratories).

Finally, the images were captured by the STELLARIS 8 confocal microscope (Leica Microsystems) using 25× water immersion objective lens. The mean diameters of the organoids were measured on the Leica Application Suite X (LAS X) software platform.

### RT-qPCR

cDNA was prepared using Primescript RT kit (Takara Inc), with 750 ng RNA per reaction. RT-qPCR was performed by using PerfeCa® SYBR® Green FAstMix® ROX (Quanta bio) in the StepOnePlus™ Real-time PCR System (Applied Biosystems). Hmbs was used as a control housekeeping gene for mouse samples, and B-Actin was used for organoids, relative expression was calculated (2^{-deltaCT} method) and compared to the mean of control group if necessary. Primers used for RT-qPCR are listed in Table 2 (mouse primers) and Table 3 (human primers).

TABLE 2
Mouse primers

| | | | SEQ |
|---|---|---|---|
| Target | Primer | Sequence | ID NO. |
| Hmbs | F | AGAAAAGTGCCGTGGGAAC | 1 |
| | R | TGTTGAGGTTTCCCCGAAT | 2 |
| Lrp2 | F | CTCGGATGAGGCAGGCTGTG | 3 |
| | R | TACAGTCCGGGTGCCCATCA | 4 |
| Atplal | F | GCTCCATCCTTCTCCACGGC | 5 |
| | R | GCATTTGCCCACAGCATCGG | 6 |
| Slc34al | F | CATCCAGTACGCCGTCACCC | 7 |
| | R | GTGGCTATCGTGGGCCACTC | 8 |
| Acta2 | F | GTCCCTCTATGCCTCTGGAC | 9 |
| (aSMA) | R | AAGGAATAGCCACGCTCAGT | 10 |
| Col1a1 | F | TGGAAACCCGAGGTATGCTT | 11 |
| | R | CATTGCATTGCACGTCATCG | 12 |
| Vimentin | F | CCCTCATTCCCTTGTTGCAG | 13 |
| | R | TAGGAGGACGAGGACACAGA | 14 |
| Serpine1 | F | TCAGTGGCCAATGGAAGACT | 15 |
| (Pai-1) | R | AACGTCATACTCGAGCCCAT | 16 |
| Ccn1 | F | AAGGGCAAGAAATGCAGCAA | 17 |
| (Cyr61) | R | CCGCATCTTCACAGTTCTGG | 18 |
| Ccn2 (Ctgf) | F | AGTGCATCCGGACACCTAAA | 19 |
| | R | GCATTTGAACTCCACTGGCA | 20 |
| Fibronectin-1 | F | TCCTCCCGTTCACCTACAAC | 21 |
| | R | ACACCACAGATGTCCGTCTT | 22 |
| Col8al | F | GTGGAGAACCTGGACCAAGA | 23 |
| | R | TACCGGGCTTTCCAATTCCT | 24 |
| Sik1 | F | AAACGAGGCTAGGCAGAAGT | 25 |
| | R | TGGACAATGTGGTGGTTGTG | 26 |
| Sik2 | F | AGTCTTTGAAGGGCAGCAGT | 27 |
| | R | AGAGCTCCACAGACAAGGAC | 28 |
| Sik3 | F | GAGCTCTTCGAAGGGAAGGA | 29 |
| | R | CATAGAGGACAACTCCAAGGC | 30 |

TABLE 3
Human primers

| | | | SEQ |
|---|---|---|---|
| Target | Primer | Sequence | ID NO. |
| B-Actin | F | CTCTTCCAGCCTTCCTTCCT | 31 |
| | R | AGCACTGTGTTGGCGTACAG | 32 |
| Serpine1 | F | CCGCCTCTTCCACAAATCAG | 33 |
| (Pai-1) | R | GTAGGGCAGTTCCAGGATGT | 34 |
| Fibronectin-1 | F | GGTACAGGGTGACCTACTCG | 35 |
| | R | GGGCTGGCTCTCCATATCAT | 36 |
| Col8al | F | CAGCAAGTACCTCACATGCC | 37 |
| | R | TTTCTTGGGTACGGCTTCCT | 38 |
| Lrp2 | F | GCGCTGCATCTCGGAAGAGT | 39 |
| | R | TGGTGGCATTGCAGTCCCTG | 40 |
| Slc17a2 | F | GCCCTTTGTGGCCTCCAGTT | 41 |
| | R | TGCGATGAGCCCAAATCCCC | 42 |
| Pth1r | F | CTGGTGGAGGGGCTGTACCT | 43 |
| | R | TTCCCGGAGCTCAAGTCCCA | 44 |

### Western Blotting

Snap-frozen kidneys were homogenized in NP-40 lysis buffer (FNN0021, Thermo) with using TissueLyserII at 30 Hz for 5 minutes. Samples were rotated at 4 deg for 20 minutes and frozen on dry ice and thawed. After centrifuging for 30 minutes at 4 deg, the supernatants were collected. After determining the protein concentration by Bradford assay, 20 g of protein was loaded for western blotting. For KGli cells, the cells were washed with cold PBS then scraped into TNT buffer (Tris-NaCl-Tween buffer, 20 mM Tris-HCl pH 8, 200 mM NaCl, 0.5% Triton X-100 containing 1X-protease inhibitor cocktail (Bimake Inc. 100X, Cat. #B14001), 1 mM DTT, 1 mM NaF, 1 mM Vanadate (New England Biolabs)). Next, samples were vortexed for 1 minute and centrifuged at 14,000g for 6 minutes at 4 deg, and the supernatant was collected.

For immunoblotting, the lysates were separated by SDS-PAGE (Bio-rad precast gels), and proteins were transferred to the nitrocellulose membranes. Then, the membranes were blocked in 5% milk in tris-buffered saline plus 0.05% Tween-20 (TBST) and incubated with primary antibody in 5% BSA-TBST overnight at 4° C. Next day, after washing, membranes were incubated with anti-rabbit HRP-coupled secondary antibody (1:2500, Cell Signaling Technology, 7074S) or anti-mouse HRP-coupled secondary antibody (1:2500, Cell Signaling, 7076S), and signals were detected with SuperSignal West Femto Maximum Sensitivity Substrate (Thermo scientific) using the iBright imager (ThermoFisher). The antibodies used are: SIK2 (1:1000, Cell Signaling, 6919), GAPDH (1:1000, Cell Signaling, 2118), αSMA (1:1000, Invitrogen, 14-9760-82), pSMAD3 (1:1000, Cell Signaling, 9520), and SMAD⅔ (1:1000, Cell Signaling, 8685).

### Staining

Hematoxylin and eosin (H+E) staining was performed on some sections using standard protocols and Masson's Trichome stain for fibrosis was performed according to the manufacturer's instructions (ab150686, abcam). Formalin-fixed paraffin embedded sections were also used for in situ hybridization according to the manufacturer instructions (ACDbio). Briefly, the sections were blocked with hydrogen peroxide for 10 minutes at room temperature, followed by antigen retrieval with Pepsin (R2283, Sigma-Aldrich) in humidified chamber in 40° C. HybEZ oven. The Sik1 (526411, ACD), Sik2 (526421, ACD), or Sik3 (526431, ACD) probe was added to the sections for 2 hours in the humidified chamber at 40° C. Then, the signal was amplified (322310, ACD) and detected using Brown DAB kit (322310, ACD).

### Bulk and Single Cell RNA-Sequencing

Isolated RNA samples from the mouse adenine diet study and human kidney organoid experiment were submitted to Novogene and bulk-RNA sequencing on NovaSeq X Plus Ilumina Novoseq Platform. Sequencing reads were mapped to the mouse reference genome (mm10/GRCm38) or human reference genome (GRCh38/hg38) using Salmon and differential gene expression analysis was performed using DESeq2.

To perform single cell RNA sequencing, mouse kidneys were harvested and immediately dissociated in liberase TH (Millipore Sigma, 05401135001) at 37 degrees for 1 hour at 600 rpm. After removing red blood cells using red blood cell lysis buffer (Sigma, R7757), the cells were further dissociated in Accumax (Stemcell Technologies, 7921) and submitted to MGH Department of Molecular Biology for gel beads in emulsion (GEM) formation and subsequent single cell RNA sequencing using the 10× Genomics Chromium 3′ droplet-based sequencing platform. The CellRanger toolkit (v6.0.2) by 10× Genomics was used to 1) align the sequencing reads to the mouse reference transcriptome (mm10). 2) quantify gene expression, resulting in gene-by-cell UMI count matrices, and 3) aggregate all samples into a single feature barcode matrix containing all the data for downstream analysis. All single cell data analysis was performed in the R software environment for statistical computing and graphics using the package Seurat.

Kidney GliI⁺ cells

KGli cells (30) were obtained from Dr. Benjamin Humphreys (Washington University, St. Louis) and cultured in Alpha MEM media (Gibco, 12571-048) with 1× Glutamax (Gibco, 35050-061) supplemented with 20% MSC-qualified FBS (Gibco, A31605-02), 10 ng/nl bFGF (Proteintech, HZ-1285), 10 ng/nl mouse EGF (proteintech, HZ-1326), and 1% antibiotic-antimycotic (Gibco, 15240-062). The cells were plated at 50,000 cells/ml in 6-well plates for western blotting, 12-well plates for RNA isolation. Before treatment, the cells were serum starved for 16-20 hours and treated with TGFβ (5 ng/ml) and SK-124 (10 M unless indicated otherwise) or verteporfin (1 μM). The cells were treated for 24 hours for gene expression and 48 hours for protein expression changes.

### Yap/Taz Responsive Luciferase Reporter Assay

To assess the signal of YAP/TAZ responsive reporter, the mouse fibroblast cell line NIH3T3 cells were transfected 24 hours after plating at 50,000 cells/ml in a 10 cm dish by using 15 μl Polyjet transfection reagent (SignaGen Laboratories, SL100688) and 5 μg of 8xGTIIC-luciferase construct. 48 hours after, the cells were then trypsinized and plated on a black 96-well plate and treated the next day for 24 hours. The luciferase and renilla signals were read using Renilla Luciferase Assay System kit (Promega, E2810) and EnVision plate reader (PerkinElmer).

### Results

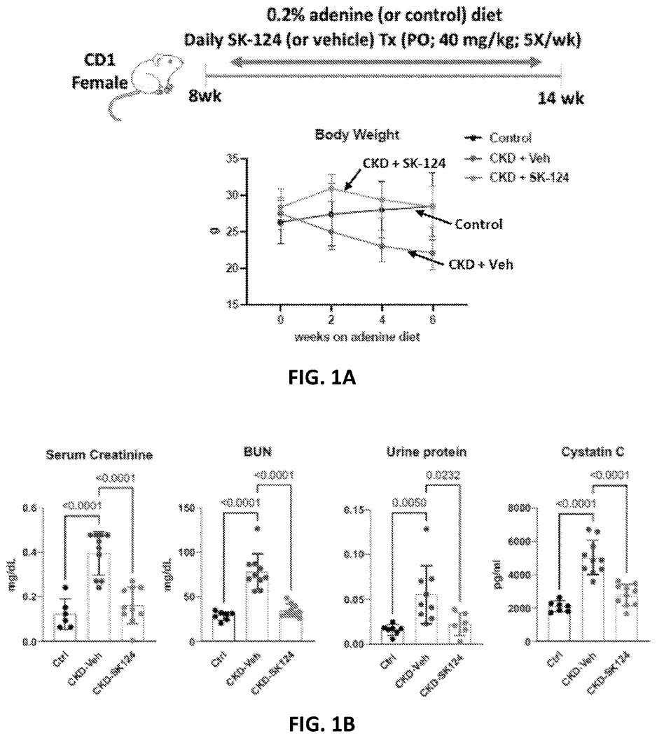

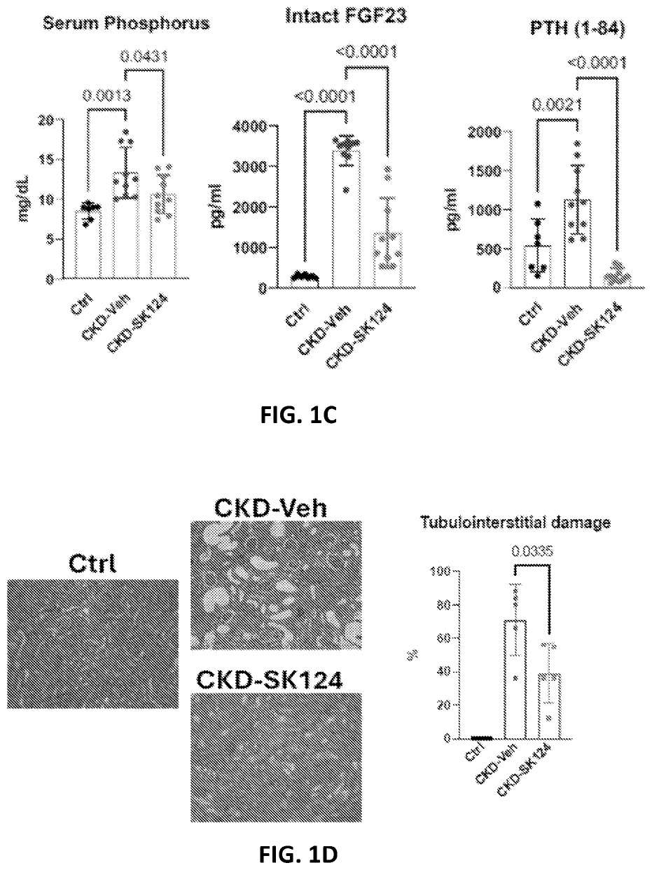

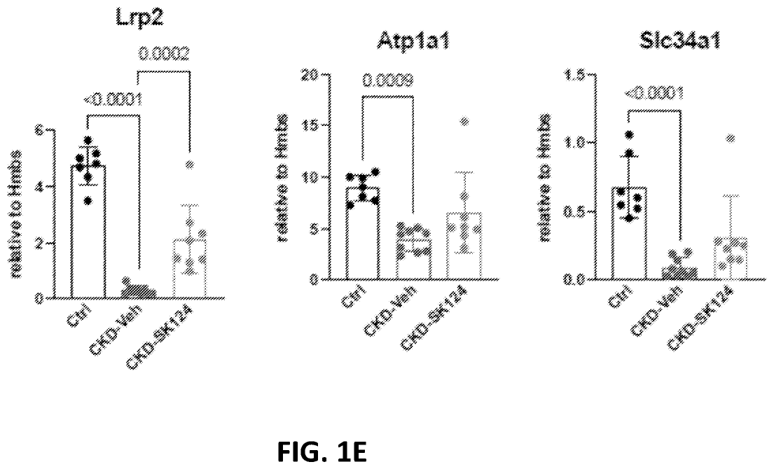

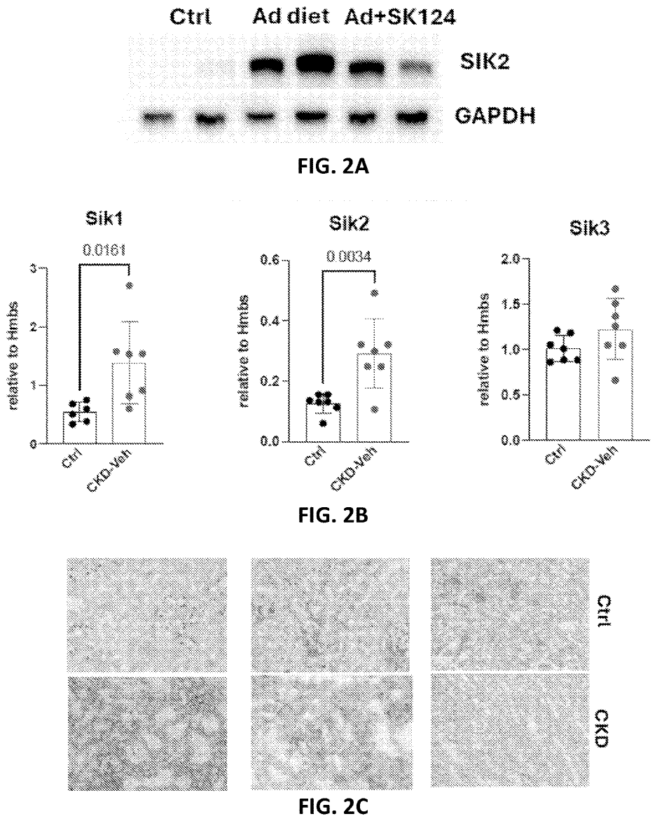

Based on our finding that salt inducible kinase inhibition stimulates Cyp27bl-driven 1,25-vitamin D production in the proximal tubule, and the fact that CKD is associated with defects in 1,25-vitamin D generation (due to excessive FGF23 and renal PTH resistance (19)), we tested whether SIK inhibitors might increase 1,25-vitamin D levels in CKD. Unexpectedly, we observed dramatic improvements in renal function and overall health following SIK inhibitor (SK-124) treatment in mice fed high adenine diet (20) for 6 weeks (FIG. 1A). In this well-established CKD model, SK-124 treatment improved body weight (FIG. 1A), reduced markers of renal dysfunction (FIG. 1B: creatinine, BUN, proteinuria, and cystatin C), and consequently improved mineral metabolism derangements including hyperphosphatemia, increased FGF-23, and secondary hyperparathyroidism (FIG. 1C). Renal protection with SK-124 treatment was associated with reduced tubule-interstitial damage (FIG. 1D), and restored expression of intact proximal tubular epithelial genes (Lrp2, Atpla1, and Slc34a1, FIG. 1E). Next, we determined SIK isoform expression in samples derived from mice with adenine diet-induced kidney disease. At the protein level, robust SIK2 upregulation is noted in kidney lysates (FIG. 2A). Whole kidney RNA RT-qPCR (FIG. 2B) showed increased Sik1 and Sik2 expression levels at this time point (6 weeks into adenine diet), with trends towards increased Sik3 expression as well. High sensitivity RNA in situ hybridization (RNAscope) confirmed these findings (FIG. 2C). Therefore, SIK isoforms are upregulated at the mRNA and protein level in this CKD model, and SIK2/SIK3 inhibitor treatment (SK-124) restores kidney function in mice with adenine diet-induced CKD.

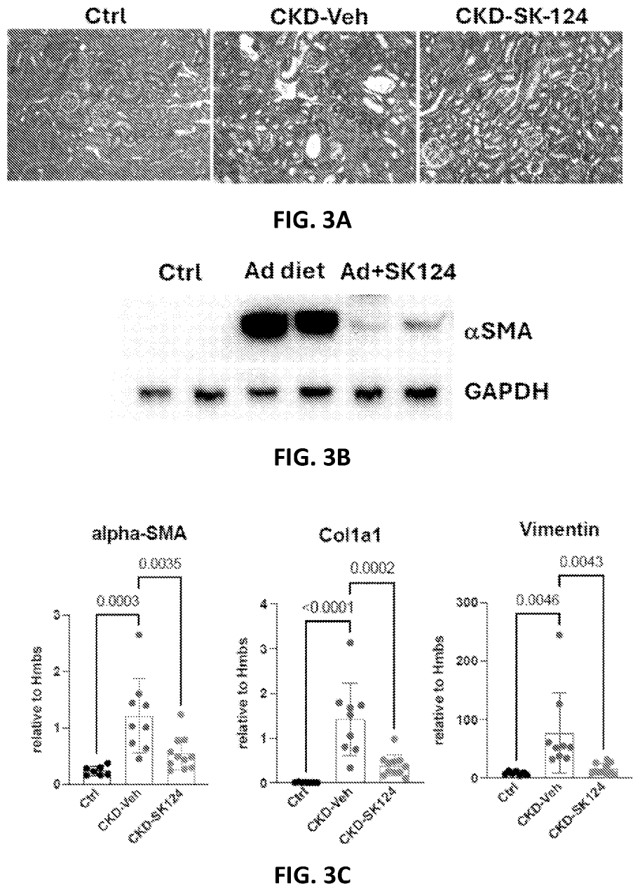

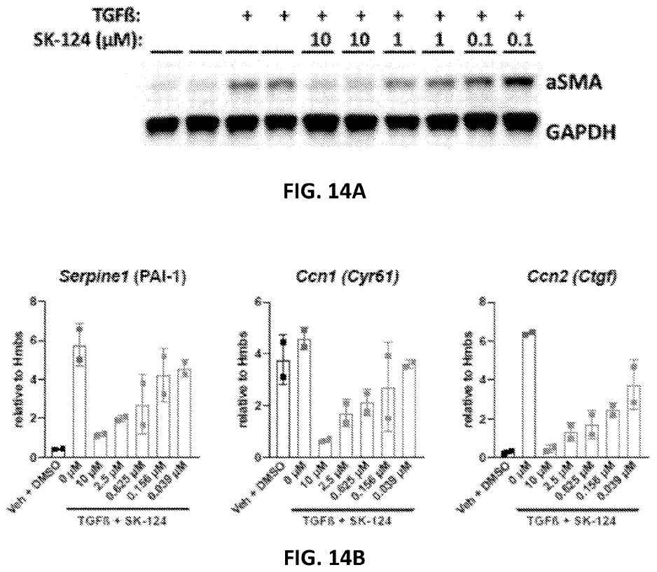

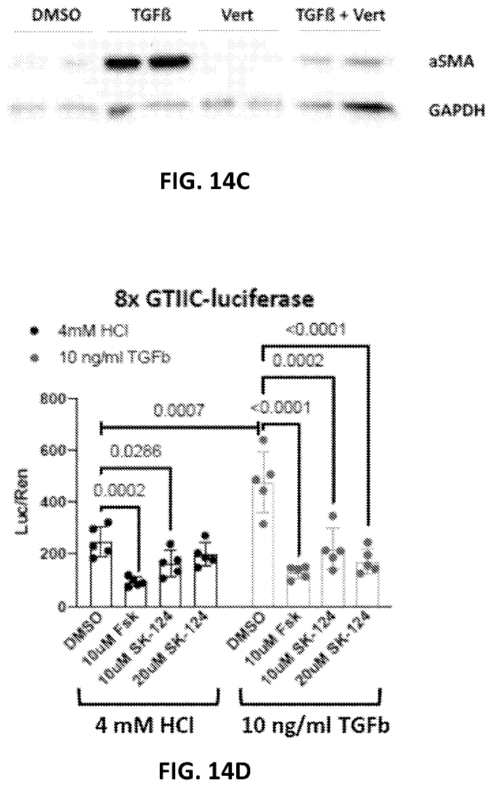

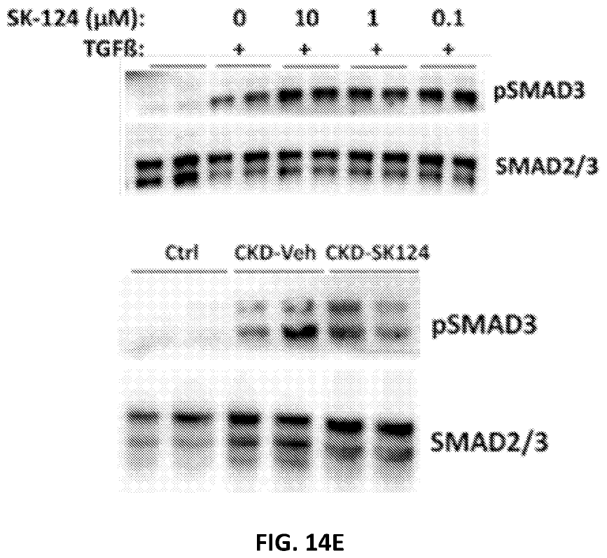

Prompted by the observation that SK-124 treatment reduced fibrosis in the adenine-induced CKD model (FIG. 3A, fibrosis noted in blue based on trichrome staining), we assessed molecular markers of renal fibrosis in this model and observed that SK-124 treatment reduced alpha-smooth muscle actin (αSMA) protein and mRNA levels, along with mRNA expression of additional fibrosis markers Colla1 and Vimentin (Vim) (FIG. 3B, 3C). At present, the relationship between changes in fibrosis following SIK2/SIK3 inhibitor treatment and preservation of renal function remains an area of active exploration. Because SK-124 treatment preserves epithelial cell integrity (FIG. 1E and below), we currently favor a model in which potential anti-fibrotic effects of SIK2/SIK3 inhibitors (FIG. 14) are not the sole cellular mechanism underlying this therapeutic effect.

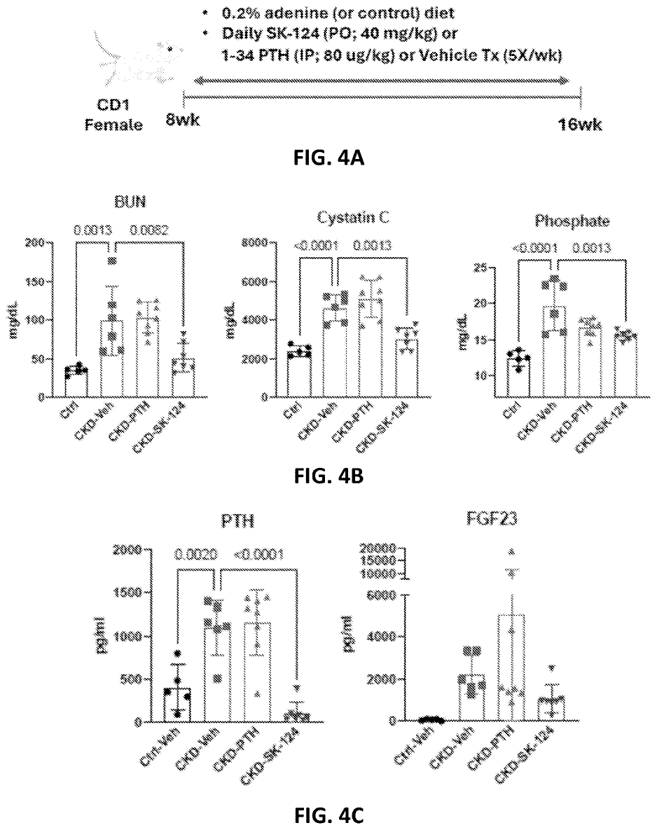

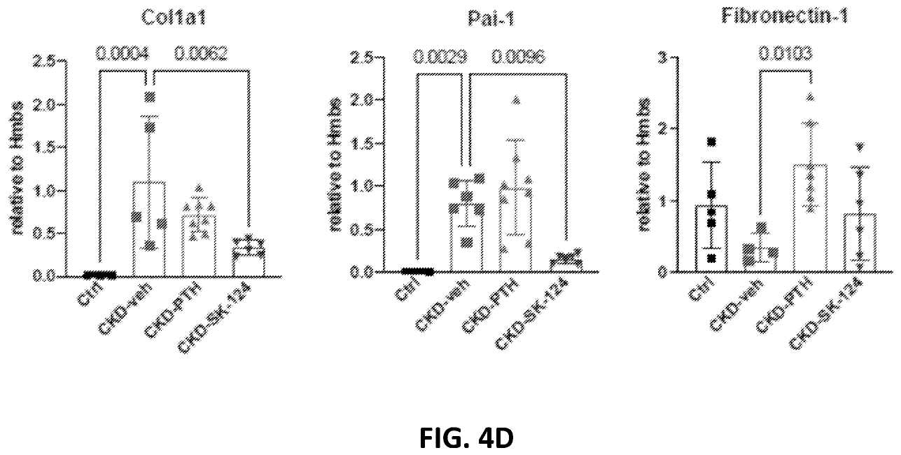

SIK2/SIK3 inhibitors can mimic parathyroid hormone (PTH) actions and therefore boost circulating 1,25-vitamin D levels (6). Since 1,25-vitamin D itself has been reported to exert anti-fibrotic effects in kidney (21), we directly compared the efficacy of SK-124 versus once daily PTH injections in an independent 8 week adenine diet CKD study (FIG. 4A). Similar to our previous results, SK-124 treatment in CKD mice again improved kidney function, reduced CKD-MBD markers, and reduced fibrosis marker gene expression. In contrast, once daily PTH treatment did not cause these effects (FIGS. 4B-4D). Therefore, the ability of SIK2/SIK3 inhibitors to improve renal function is independent of their actions downstream of the PTH receptor.

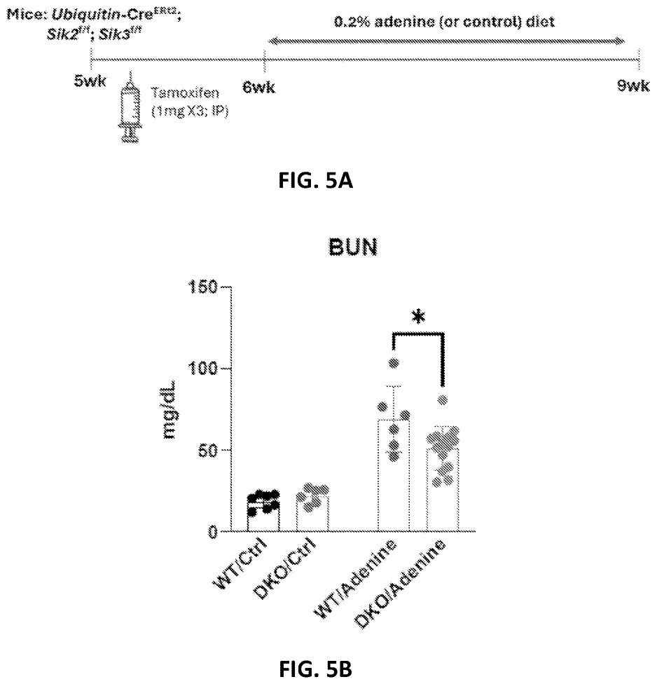

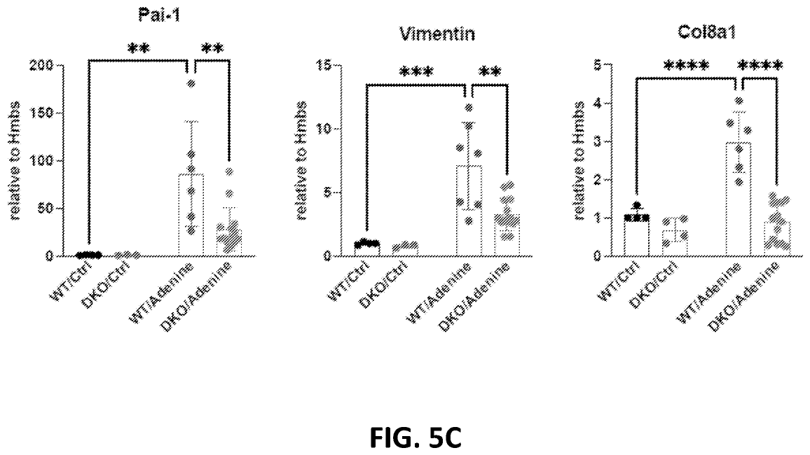

Although SK-124 is a specific SIK2/SIK3 inhibitor, this kinase inhibitor may have weak ‘off-target’ actions at other kinases. Therefore, we used global/inducible SIK2/SIK3 knockout (double knockout. DKO) mice (12) to complement our pharmacologic studies. SIK2/SIK3 were deleted in all cells in 5-week-old mice, and then control and DKO animals were fed either control or adenine diets (FIG. 5A). Similar to our findings with SK-124 treatment, SIK2/SIK3 gene deletion improved renal function (FIG. 5B) and reduced expression of injury markers (FIG. 5C). Based on these genetic findings, the therapeutic targets of SK-124 are most likely SIK2/SIK3.

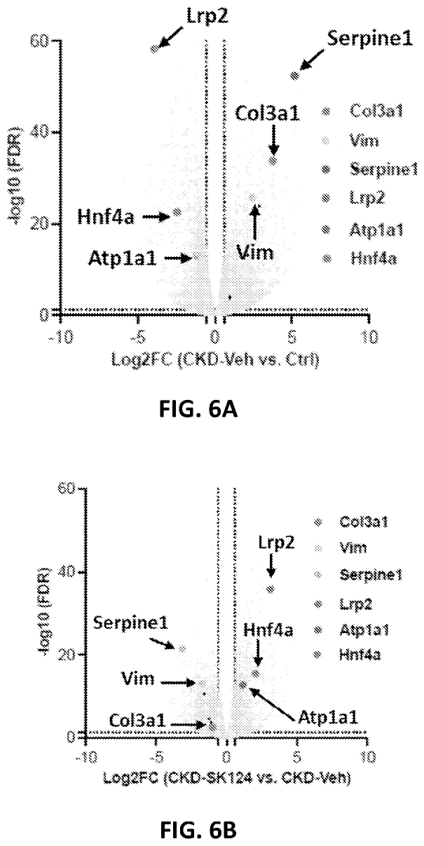

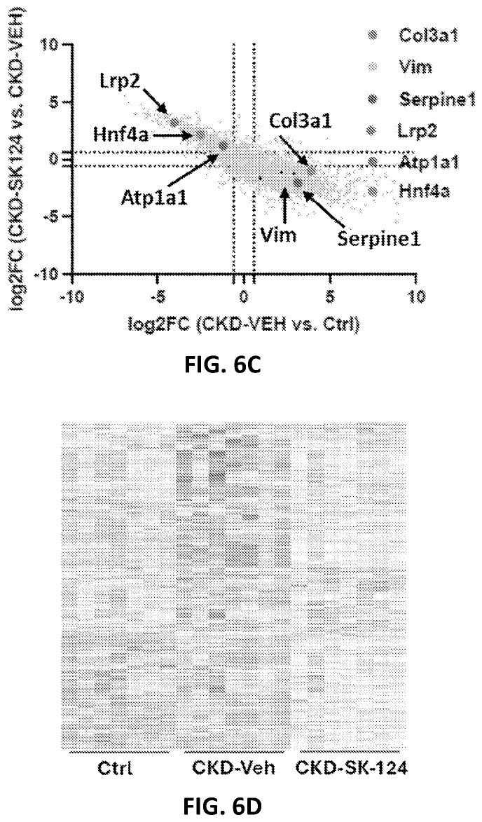

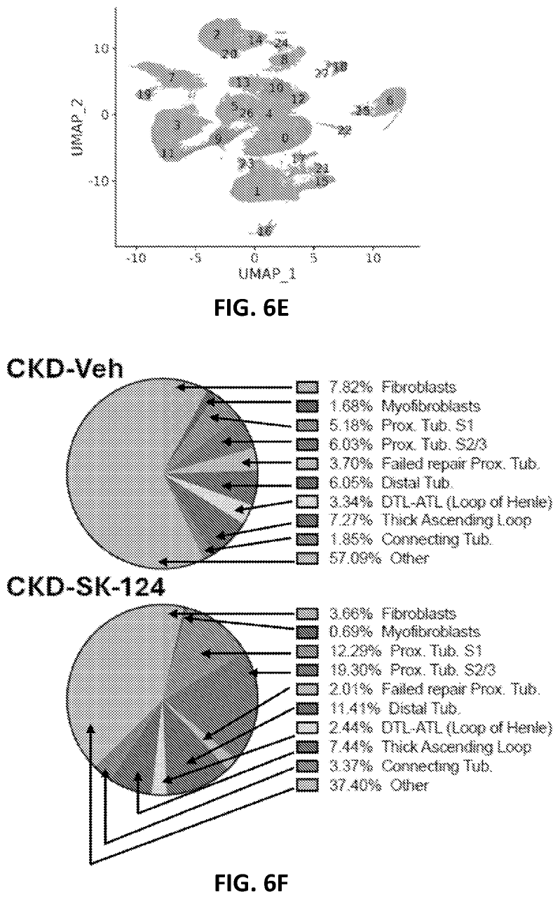

Next, we used transcriptomic profiling to define the molecular correlates of improved renal function following SK-124 treatment in the adenine CKD model. First, ‘tbulk’ RNA-sequencing defined genes regulated by control versus adenine diet (FIG. 6A). As expected, injury markers (Co/3a1, Vim, Serpinel) were upregulated, and markers of intact proximal epithelial cells (Lrp2, Atpla1, and Hnf4a) were reduced in this CKD model. Next, we assessed the effects of SK-124 treatment in CKD mice (FIG. 6B); remarkably, this analysis revealed that CKD-associated injury markers were suppressed by SK-124 treatment, and SK-124 treatment preserved expression of markers of epithelial cell integrity. Scatterplot (FIG. 6C) and heat map (FIG. 6D) analysis confirmed overall trends that patterns of dysregulated gene expression in CKD are reversed with SK-124 treatment. To complement these studies using whole kidney RNA-sequencing, we also performed single cell RNA-sequencing in CKD mice treated with vehicle or SK-124 (FIG. 6E). Consistent with ‘bulk’ RNA-sequencing results, SK-124 treatment reduced injury markers (failed repair proximal tubule cells, fibroblasts, and myofibroblasts) and increased numbers of healthy proximal tubule cells (FIG. 6F).

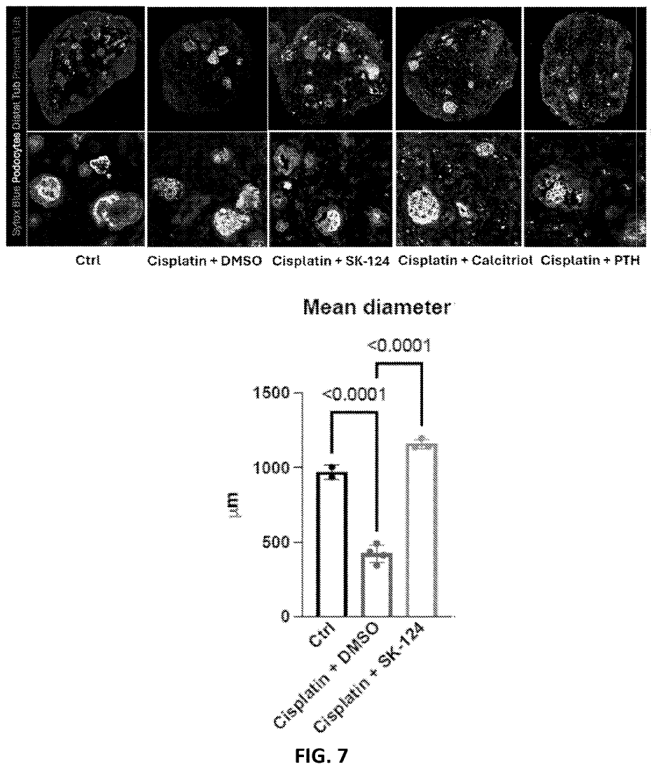

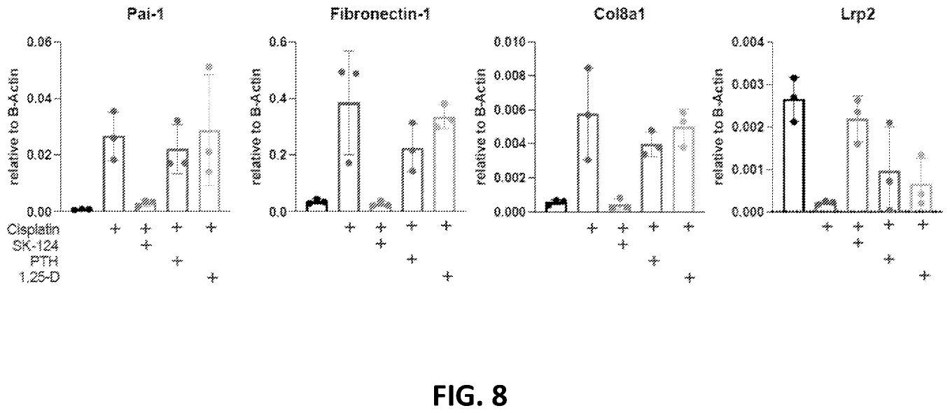

Although rodent models have been tremendously useful to explore fundamental biology and disease mechanisms, many pathways and compounds studied in mice fail to progress into successful therapies. Although many factors likely account for this phenomenon (22, 23), contemporaneous use of complementary, human-relevant models is needed. To this end, human pluripotent stem cell-derived organoids represent a powerful confirmatory system (24). Advances in stem cell and developmental biology have enabled generation of robust human kidney organoids (25, 26). Injury models in such human cell-derived systems enable modeling of repair/fibrosis processes, as recently reported (27) using cisplatin-induced renal injury to study epithelial cell repair and initiation of fibrosis in response to tubular injury. Human kidney organoids were injured with intermittent cisplatin (5 treatments) in the presence or absence of SK-124, calcitriol (1,25-vitamin D), or PTH. Analysis of organoid cellular composition by multi-channel confocal microscopy revealed expected patterns of proximal tubular loss following cisplatin treatment. While calcitriol and PTH treatment had minimal effects on patterns of cisplatin-induced injury, SK-124 treatment was associated with preserved proximal tubule cells and reduced cisplatin-induced organoid atrophy (FIG. 7). Moreover, SK-124 treatment blocked cisplatin-induced expression of injury markers (Pai-1, Fibronectin-1, and Col8a1), preserved expression of the proximal tubule viability gene Lrp2 (FIG. 8).

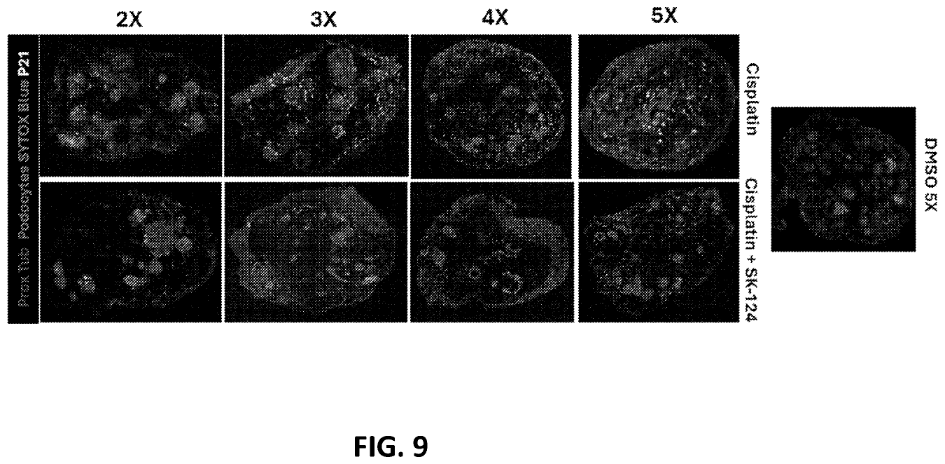

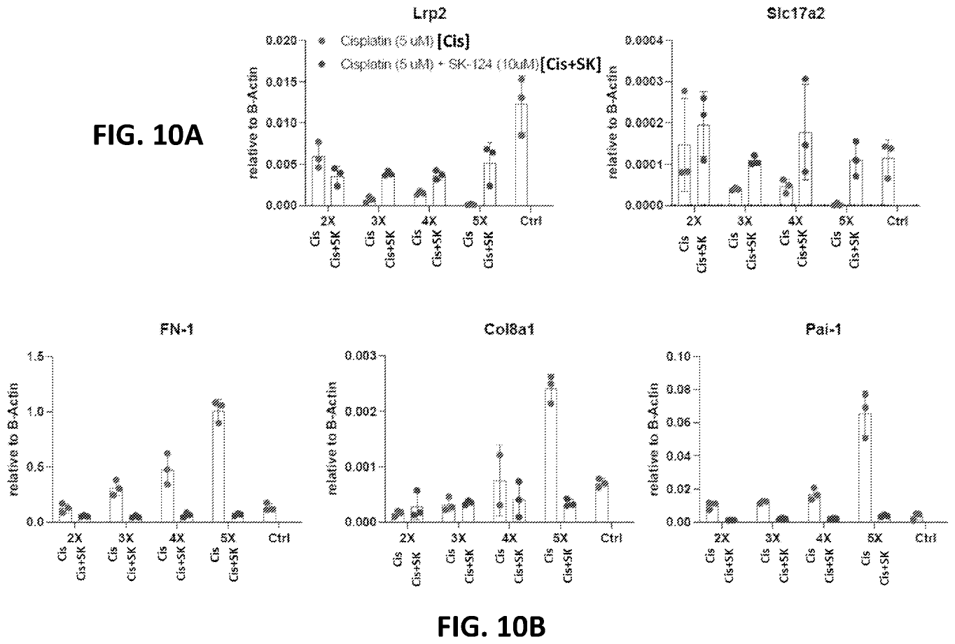

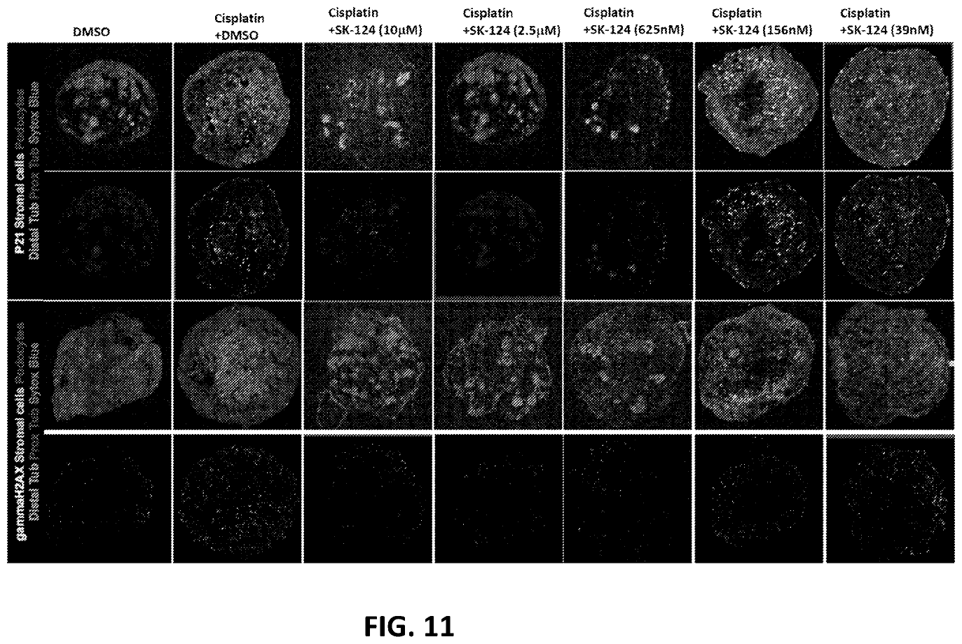

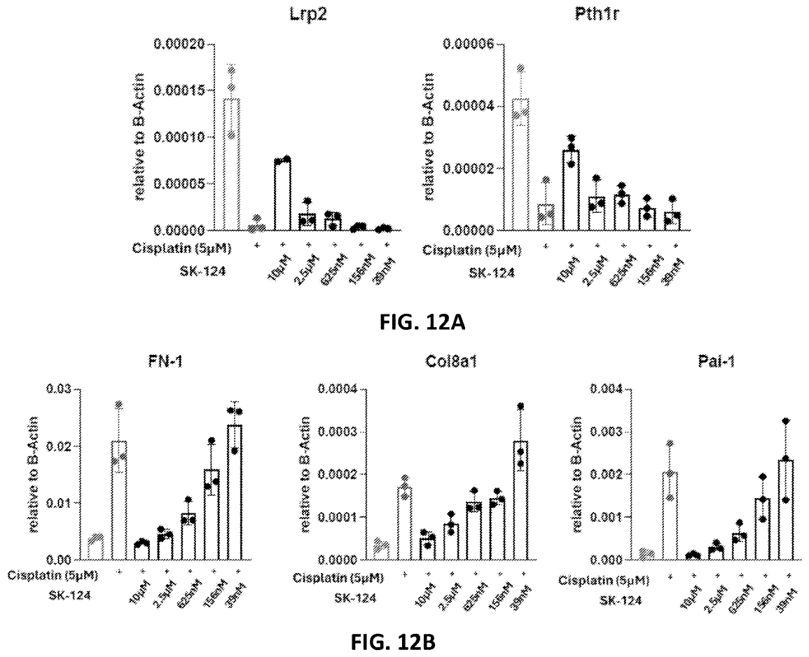

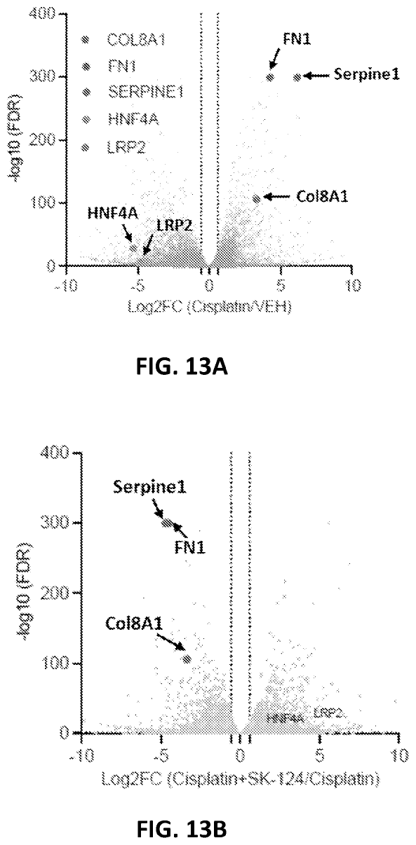

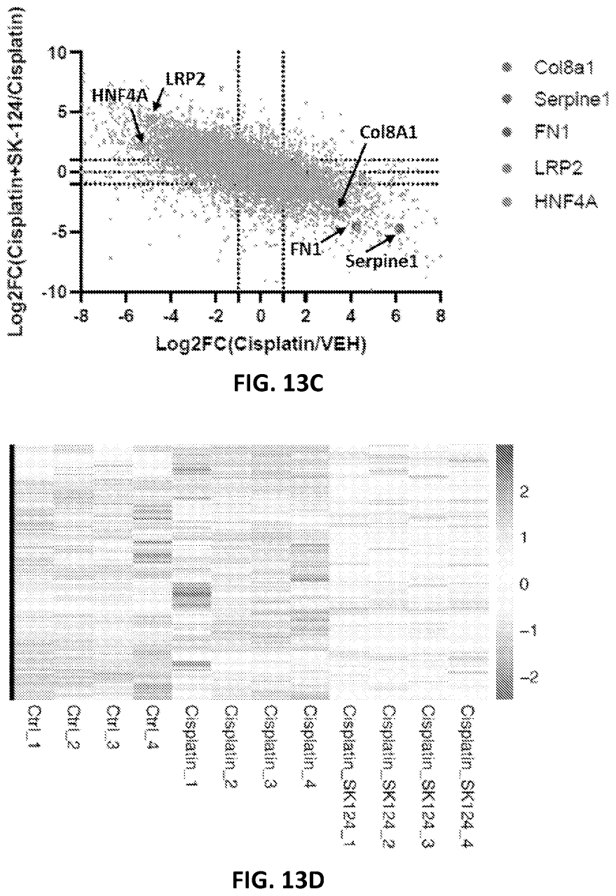

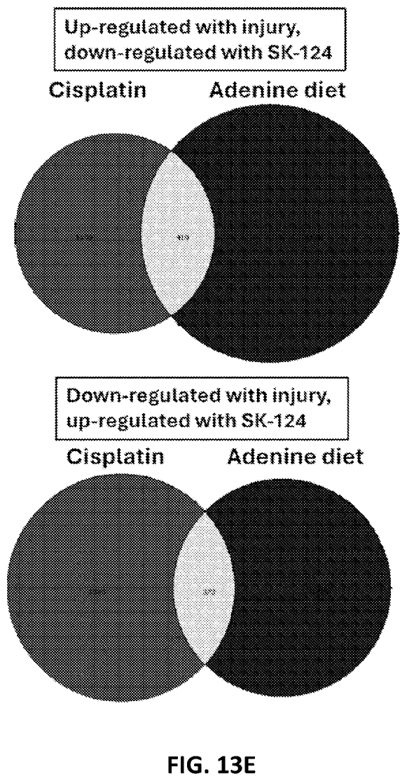

Expression of the cyclin dependent kinase inhibitor P21 in injured proximal tubule epithelial cells is a hallmark of early tubular injury in vivo (28), and is observed in cisplatin-injured organoids (27). Therefore, we assessed P21 induction during the course of cisplatin injury in the presence or absence of concomitant SK-124 treatment. While robust P21 (top) and gammaH2AX (bottom) induction was noted in organoids exposed to cisplatin/vehicle, these injury markers were markedly reduced by concurrent SK-124 treatment (FIG. 9). RNA isolated from this cisplatin+/−SK-124 time course experiment demonstrate that SK-124 improved expression of intact epithelial markers (Lrp2 and Slc17a2) and suppressed expression of injury markers (Fibronectin-1, Col8a1, and Pai-1) (FIGS. 10A, 10B). Dose response experiments in cisplatin-treated kidney organoids (FIG. 11) again revealed robust cisplatin-induced P21 induction and loss of LTL-positive proximal tubule cells which was blocked by SK-124 treatment in a dose-dependent manner. RNA isolated from this dose response organoid experiment revealed expected suppression of proximal tubule markers (Lrp2 and Pth1r) following cisplatin treatment with dose-dependent restoration by SK-124 treatment (FIG. 12A) which was accompanied by cisplatin-induced induction of injury markers (Fibronectin-1, Col8al, and Pai-1) which were suppressed by this SIK2/SIK3 inhibitor (FIGS. 12A, 12B). The fact that therapeutic effects of SK-124 are noted in the injury model at doses below 1 μM strongly supports an ‘on-target’ mechanism of this SIK2/SIK3 inhibitor. Finally, RNA-sequencing was performed in human kidney organoids to define the transcriptomic effects of SK-124 treatment in cisplatin-induced injury. As expected, injury markers (Col8al, Fibronectin-1, and Serpinel) were induced by cisplatin versus vehicle, and reduced by SK-124 in cisplatin-treated organoids. In contrast, markers of intact proximal integrity (Lrp2 and Hnf4a) were reduced by cisplatin and restored by SK-124 treatment (FIGS. 13A, 13B). Scatterplot (FIG. 13C) and heatmap (FIG. 13D) analysis again demonstrated that overall effects of kidney injury are ameliorated by SIK2/SIK3 inhibitor treatment in this human system. Finally, integration of genes induced by kidney injury and rescued by SK-124 treatment between the mouse adenine diet and human cisplatin organoid studies revealed a robust core set of 419 conserved genes that are both induced with injury and reduced by SK-124 treatment and 373 genes whose expression is both suppressed by injury and restored by SK-124 treatment (FIG. 13E).

### DISCUSSION

Taken together, these findings demonstrate that SIK2/SIK3 inhibition improves phenotypic and transcriptomic profiles in the setting of kidney injury across different CKD models and species. Accordingly, these findings using complementary human and mouse models demonstrate that SIK2/SIK3 inhibitors bear potential to treat chronic kidney disease.

### REFERENCES

1. August P. Chronic Kidney Disease—Another Step Forward. The New England journal of medicine. 2023; 388(2):179-80.
2. Wein M N, Foretz M, Fisher D E, Xavier R J, Kronenberg H M. Salt-Inducible Kinases: Physiology, Regulation by cAMP, and Therapeutic Potential. Trends Endocrinol Metab. 2018; 29(10):723-35.
3. Jagannath A, Taylor L, Ru Y, Wakaf Z, Akpobaro K, Vasudevan S, Foster R G. The multiple roles of salt-inducible kinases in regulating physiology. Physiol Rev. 2023; 103(3):2231-69.
4. Wein M N, Liang Y, Goransson O, Sundberg T B, Wang J, Williams E A, et al. SIKs control osteocyte responses to parathyroid hormone. Nature communications. 2016:7:13176.
5. Nishimori S, O'Meara M J, Castro C D, Noda H, Cetinbas M, da Silva Martins J, et al. Salt-inducible kinases dictate parathyroid honnone 1 receptor action in bone development and remodeling. J Clin Invest. 2019; 129(12):5187-203.
6. Yoon S H, Meyer M B, Arevalo C, Tekguc M, Zhang C, Wang J S, et al. A parathyroid hormone/salt-inducible kinase signaling axis controls renal vitamin D activation and organismal calcium homeostasis. J Clin Invest. 2023;133(9).
7. Sato T, Andrade C D C, Yoon S H, Zhao Y, Greenlee W J. Weber P C. et al. Structure-based design of selective, orally available salt-inducible kinase inhibitors that stimulate bone formation in mice. Proceedings of the National Academy of Sciences of the United States of America. 2022;119(50):e2214396119.
8. Takemori H, Doi J, Horike N, Katoh Y, Min L, Lin X Z, et al. Salt-inducible kinase-mediated regulation of steroidogenesis at the early stage of ACTH-stimulation. J Steroid Biochem Mol Biol. 2003;85(2-5):397-400.
9. Henriksson E, Jones H A, Patel K, Peggie M, Morrice N, Sakamoto K, Goransson O. The AMPK-related kinase SIK2 is regulated by cAMP via phosphorylation at Ser358 in adipocytes. Biochem J. 2012; 444(3):503-14.
10. Sonntag T, Vaughan J M, Montminy M. 14-3-3 proteins mediate inhibitory effects of cAMP on salt-inducible kinases (SIKs). FEBS J. 2018; 285(3):467-80.
11. Darling N J. Cohen P. Nuts and bolts of the salt-inducible kinases (SIKs). Biochem J. 2021:478(7):1377-97.
12. Tang C C, Castro Andrade C D, O'Meara M J, Yoon S H, Sato T, Brooks D J, et al. Dual targeting of salt inducible kinases and CSF1R uncouples bone formation and bone resorption. Elife. 2021;10.
13. Sakamoto K, Bultot L, Goransson O. The Salt-Inducible Kinases: Emerging Metabolic Regulators. Trends Endocrinol Metab. 2018; 29(12):827-40.
14. Wein M N, Foretz M, Fisher D E, Xavier R J, Kronenberg H M. Salt-Inducible Kinases: Physiology, Regulation by cAMP, and Therapeutic Potential. Trends Endocrinol Metab. 2018.
15. Fan D, Yang H, Mao W, Rask P J, Pang L, Xu C, et al. A Novel Salt Inducible Kinase 2 Inhibitor, ARN-3261, Sensitizes Ovarian Cancer Cell Lines and Xenografts to Carboplatin. Cancers (Basel). 2021;13(3).
16. Rak M, Tesch R, Berger L M, Shevchenko E, Raab M, Tjaden A, et al. Shifting the selectivity of pyrido[2,3-d]pyrimidin-7(8H)-one inhibitors towards the salt-inducible kinase (SIK) subfamily. Eur J Med Chem. 2023:254:115347.
17. Zhu W, Liu X, Li Q, Gao F, Liu T, Chen X, et al. Discovery of novel and selective SIK2 inhibitors by the application of AlphaFold structures and generative models. Bioorg Med Chem. 2023; 91:117414.
18. Tesch R, Rak M, Raab M, Berger L M. Kronenberger T, Joerger A C, et al. Structure-Based Design of Selective Salt-Inducible Kinase Inhibitors. J Med Chem. 2021; 64(12):8142-60.
19. Hruska K A, Sugatani T, Agapova O, Fang Y. The chronic kidney disease —Mineral bone disorder (CKD-MBD): Advances in pathophysiology. Bone. 2017; 100:80-6.
20. Jia T, Olauson H, Lindberg K, Amin R, Edvardsson K, Lindholm B, et al. A novel model of adenine-induced tubulointerstitial nephropathy in mice. B M C Nephrol. 2013; 14:116.
21. I to I, Waku T, Aoki M, Abe R, Nagai Y. Watanabe T, et al. A nonclassical vitamin D receptor pathway suppresses renal fibrosis. J Clin Invest. 2013; 123(11):4579-94.
22. Drucker D J. Never Waste a Good Crisis: Confronting Reproducibility in Translational Research. Cell metabolism. 2016:24(3):348-60.
23. von Herrath M, Pagni P P, Grove K, Christoffersson G, Tang-Christensen M, Karlsen A E, Petersen J S. Case Reports of Pre-clinical Replication Studies in Metabolism and Diabetes. Cell metabolism. 2019; 29(4):795-802.
24. Kim J, Koo B K, Knoblich J A. Human organoids: model systems for human biology and medicine. Nat Rev Mol Cell Biol. 2020; 21(10):571-84.
25. Morizane R, Lam A Q, Freedman B S, Kishi S, Valerius M T, Bonventre JV. Nephron organoids derived from human pluripotent stem cells model kidney development and injury. Nat Biotechnol. 2015; 33(11):1193-200.
26. Takasato M, Er P X, Chiu H S, Maier B, Baillie G J. Ferguson C, et al. Kidney organoids from human iPS cells contain multiple lineages and model human nephrogenesis. Nature. 2015:526(7574):564-8.
27. Gupta N, Matsumoto T, Hiratsuka K, Garcia Saiz E, Galichon P, Mivoshi T, et al. Modeling injury and repair in kidney organoids reveals that homologous recombination governs tubular intrinsic repair. Sci Transl Med. 2022:14(634):eabj4772.
28. Koyano T, Namba M, Kobayashi T, Nakakuni K, Nakano D, Fukushima M, et al. The p21 dependent G2 arrest of the cell cycle in epithelial tubular cells links to the early stage of renal fibrosis. Sci Rep. 2019;9(1):12059.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating chronic kidney disease (CKD) in a subject in need thereof, the method comprising administering to the subject a therapeutically effective amount of an inhibitor of one or more salt inducible kinases (SIKs).

2. The method of claim 1, wherein the subject does not have acute kidney injury (AKI) and/or CKD Mineral and Bone Disorder (CKD-MBD).

3. (canceled)

4. The method of claim 1, wherein the subject has early-stage CKD or intermediate-stage CKD.

5. (canceled)

6. The method of claim 1, wherein the method results in one or more of:
reduction in one or more symptoms of CKD in the subject;
reduced expression of one or more markers of renal dysfunction in the subject;
reduced expression of one or more fibrosis markers in the subject;
reduced expression of one or more renal injury markers in the subject;
reduced expression of one or more proximal tubule injury markers in the subject;
increased expression of one or more markers of intact proximal epithelial cells in the subject;
preservation of epithelial cell integrity in the subject;
increased number of healthy proximal tubule cells in the subject; and/or
improved renal function in the subject,
as compared to before the inhibitor is administered to the subject.

7. The method of claim 6, wherein the markers of renal dysfunction comprises one or more of creatinine, BUN, proteinuria, and cystatin C; the fibrosis marker comprises one or more of alpha-smooth muscle actin (αSMA), Collal, and Vimentin; the renal injury marker comprises one or more of Col3al, Vim, Serpinel, Pai-1, Fibronectin-1, and Col8al; the proximal tubule injury marker comprises p21; and/or the marker of intact proximal tubular epithelial cells comprises one or more of Lrp2, Atplal, Slc34al, Hnf4a, and Slc17a2.

8. The method of claim 1, further comprising administering to the subject one or more additional therapeutic agents: optionally wherein the one or more additional therapeutic agents comprises an ACE inhibitor, an angiotensin II receptor blocker, a lipid control agent, a D-vitamin supplementation, a phosphate control agent, an anemia control agent, an acidosis prevention agent, and/or a uric acid control agent.

9. (canceled)

10. A method for reducing the risk of occurrence or delaying the occurrence of chronic kidney disease (CKD) in a subject in need thereof, the method comprising administering to the subject a therapeutically effective amount of an inhibitor of one or more salt inducible kinases (SIKs).

11. The method of claim 10, wherein the subject is at risk for developing CKD; optionally wherein the subject has one or more of diabetes, high blood pressure, heart disease, family history of CKD, and obesity.

12. (canceled)

13. The method of claim 1, wherein one or more doses of the inhibitor is administered to the subject.

14. The method of claim 1, wherein the inhibitor is administered to the subject multiple times at daily, weekly, or monthly intervals.

15. The method of claim 1, wherein the inhibitor is an inhibitor of one or more of SIK2 and SIK3.

16. The method of claim 1, wherein the inhibitor is a compound that is a selective SIK2/SIK3 inhibitor, or a pharmaceutically acceptable salt thereof.

17. The method of claim 1, wherein the inhibitor is 2-[2,6-Dimethoxy-4-[7-(1-methylpyrazol-4-yl)imidazo[1,2-a]pyridin-3-yl]phenyl]-5-ethyl-1,3,4-oxadiazole, or a pharmaceutically acceptable salt thereof.

18. The method of claim 1, wherein the inhibitor is a compound having the structure: 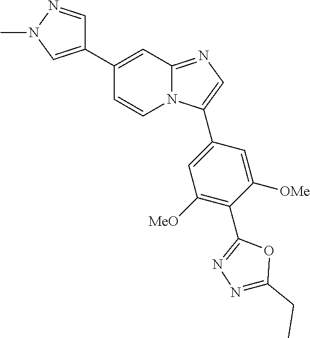 [SMILES: CCc1nnc(-c2c(OC)cc(-c3cnc4cc(-c5cnn(C)c5)ccn34)cc2OC)o1]
or a pharmaceutically acceptable salt thereof.

19. The method of claim 1, wherein the inhibitor is an inhibitory nucleic acid that targets a SIK2/SIK3 transcript; optionally wherein the inhibitory nucleic acid is selected from the group consisting of antisense oligonucleotides, small interfering RNAs (siRNAs), and small hairpin RNAs (shRNAs).

20. (canceled)

21. The method of claim 19, wherein the inhibitory nucleic acid is modified.

22. The method of claim 21, wherein the inhibitory nucleic acid comprises a modified backbone; optionally wherein the modified backbone comprises an amide or morpholino backbone.

23. (canceled)

24. The method of claim 21, wherein the inhibitory nucleic acid comprises one or more modified nucleosides; optionally wherein the inhibitory nucleic acid comprises at least one locked nucleoside.

25. (canceled)

26. The method of claim 1, wherein the subject is a human.

27-36. (canceled)
